(19) United States
(12) Patent Application Publication
(10) Pub. No.: US 2020/0147118 A1
(43) Pub. Date: May 14, 2020

(54) COMPOSITIONS AND METHODS FOR DETECTION, RISK ASSESSMENT AND TREATMENT OF DIABETES, OBESITY, AND INFLAMMATION

(71) Applicant: OTTAWA HEART INSTITUTE RESEARCH CORPORATION, Ottawa (CA)

(72) Inventors: Katey Rayner, Ottawa (CA); Denuja Karunakaran, North Balwyn (AU); Ruth McPherson, Ottawa (CA)

(21) Appl. No.: 16/186,423

(22) Filed: Nov. 9, 2018

(51) Int. Cl. A61K 31/7088; A61P 3/04; A61P 3/10; A61P 29/00; C12N 15/113; A61K 48/00; G01N 33/50

(52) U.S. Cl. CPC A61K 31/7088; A61P 3/04; A61P 3/10; G01N 33/5041; C12N 15/113; A61K 48/0016; A61P 29/00

(57) ABSTRACT

Provided are compounds and compositions associated with obesity, diabetes and inflammation and methods of treating the same.

### FIELD OF INVENTION

The present invention relates to compositions and methods for the detection, risk assessment and treatment of diabetes, obesity, and inflammation. More specifically, the present invention relates to nucleic acids and inhibitors of RIPK1 for the detection, risk assessment and treatment of diabetes, obesity, inflammation.

### BACKGROUND OF THE INVENTION

Obesity is a major public health burden worldwide, greatly increasing the risk of diabetes, cardiovascular diseases and cancer. Obesity and associated insulin resistance are characterized by chronic low-grade inflammation driven by the cooperation of the innate immune system and dysregulated metabolism in adipose tissue and other metabolic organs. Obesity can be characterized by the interplay of dysregulated energy balance and non-resolving inflammation. Subcutaneous and visceral adipose tissue depots expand over time by the differentiation of stromal stem cells to mature adipocytes and the esterification of free fatty acids to glycerol for storage in lipid droplets. As these adipose depots expand, the synergistic conversion of tissue-resident macrophages to an M1-like phenotype and expression of cytokines and adipokines by adipocytes promotes a pro-inflammatory state^{1,2}. This metabolically-triggered systemic inflammation, particularly in the visceral adipose depots, increases the risk of developing other diseases, including atherosclerosis, type 2 diabetes and cancer. Accordingly, large clinical trials are underway to target innate immune signals to treat type 2 diabetes and reduce cardiovascular disease.

Although strong environmental drivers can lead to excess fat accumulation, studies have shown the possible heritable nature of body weight³. Both monogenic and polygenic drivers of obesity are commonly associated with the leptin/melanocortin pathways, which acts as a conduit between the central nervous system and peripheral adipose and muscle tissue. Large genome-wide association studies have revealed common variants in the FTO locus, which contains the coding region for the alpha-ketoglutarate dependent dioxygenase enzyme, that associate with the risk of obesity⁴⁻⁷. Individuals harbouring the risk allele of this locus have increased calorie intake⁸ and mouse studies suggest this distal enhancer regulates mitochondrial metabolism in adipose tissue independent of FTO expression⁹. Similarly, variants near MC4R associate with early onset obesity due to defects in the appetite control pathway and signals of satiety in the brain^{10,11}. Despite the strong pathophysiological evidence that inflammation drives obesity, to date there have been no genetic associations between inflammatory pathways and obesity in humans. While an environment of excess caloric intake and decreased energy expenditure promotes adipose tissue expansion, there remains a strong but poorly understood genetic component to obesity. Heritable variants in genes involved in appetite control (e.g. leptin, melanocortin-4 receptor) and resting energy expenditure (e.g. FTO) pathways predispose individuals to risk of obesity and weight gain²⁷.

There is a need in the art to identify genetic markers associated with weight gain. Further, there is a need in the art for genetic markers associated with obesity, diabetes and inflammation. Further, there is a need in the art for genetic diagnostic markers for weight gain that provide physicians and other health care professionals with the opportunity to provide educated decisions for prescribing medications in treatment regimens. Moreover, there is a need in the art for personalized medicine approaches that lower the risk of developing weight gain and related ailments such as diabetes and cardiovascular disease. Previous genetic association studies have not found variants in inflammatory pathways that associate with risk of obesity, confounding the understanding of whether activation of inflammation truly promotes adiposity and its associated metabolic dysfunction.

### SUMMARY OF THE INVENTION

According to an embodiment of the present invention, there is provided an isolated nucleic acid associated with increased risk of weight gain, obesity, inflammation, diabetes or combination thereof, the nucleic acid comprising:

a) at least 7 consecutive nucleotides of SEQ ID NO: 1 and comprising “CAGTC” at position 26, or a sequence complementary thereto;
b) at least 7 consecutive nucleotides of SEQ ID NO: 2 and comprising G at position 27, or a nucleotide sequence that is complementary thereto;
c) at least 7 consecutive nucleotides of SEQ ID NO: 3 and comprising T at position 26, or a sequence complementary thereto;
d) at least 7 consecutive nucleotides of SEQ ID NO: 4 and comprising C at position 26, or a sequence complementary thereto;
e) at least 7 consecutive nucleotides of SEQ ID NO: 5 and comprising C at position 26, or a sequence complementary thereto;
f) at least 7 consecutive nucleotides of SEQ ID NO: 6 and comprising G at position 26, or a sequence complementary thereto;
g) at least 7 consecutive nucleotides of SEQ ID NO: 7 and comprising “TTA” at position 26, or a sequence complementary thereto;
h) at least 7 consecutive nucleotides of SEQ ID NO: 8 and further comprising TTTAGAAAGTA at position 26, or a sequence complementary thereto; or
i) at least 70% identity to the nucleotide sequence defined in any one of a)-h).
In such an embodiment, for nucleic acid sequences that are not 100% identical to the above recited SEQ ID NOs, the polymorphism at the polymorphic site is retained. For example, the invention contemplates embodiments wherein a nucleotide sequence is 70% identical to 50 consecutive nucleotides of SEQ ID NO:2 but comprises a G at relative position 27 shown in SEQ ID NO:2 in the sequence of interest (the G at relative position 27 may occur at a different position in the sequence of interest). Thus, as will be understood by a person of skill in the art, the definitions of the polymorphisms and their locations are meant to be relative to SEQ ID Nos:1-8.

In a further embodiment, the nucleic acids as described herein and throughout are labeled, for example with a radioactive label, a fluorophore or any other marker known in the art to facilitate identification.

The invention also provides a nucleic acid as described above and herein throughout which is greater than 7 nucleotides in length, for example, but not limited to 8, 9, 10, 11, 12, 13, 14, 15, 16, 17, 18, 19, 20, 21, 22, 23, 24, 25, 26, 27, 28, 29, 30, 40, 50, 60, 70, 80, 90, 100 or more nucleotides. Further the length may be defined by a range of any two values noted above or any two values therein between. For example, but not to be considered limiting in any manner, the invention contemplates nucleic acids having a size range of between 11 and 25, or between 41 and 95. Ranges outside those specifically described are also contemplated.

The present invention also contemplates a nucleic acid that hybridizes to the nucleic acid defined above or its complement under stringent hybridization conditions.

In a further embodiment, there is provided a nucleic acid as described above and throughout for reducing the expression of RIPK1 in a cell. In a further embodiment, the cell is an in vitro cell. In a separate embodiment, the cell is an in vivo cell.

In a further embodiment there is provided a double-stranded nucleic acid that comprises:

a sense strand; and
an antisense strand comprising a region that is substantially complementary to the sequence listed in any one of SEQ ID NOS: 1-8, wherein said region of complementarity is at least 7 contiguous nucleotides in length and wherein said nucleic acid, upon contact with a cell expressing said RIPK1 gene, reduces expression of said RIPK1 gene. In a further embodiment, the double-stranded nucleic acid comprises a sequence that is substantially complementary to SEQ ID NO:1, or SEQ ID NO:2, or SEQ ID NO:3, or SEQ ID NO:4, or SEQ ID NO:5, or SEQ ID NO:6, or SEQ ID NO:7 or SEQ ID NO:8. In a preferred embodiment, which is not meant to be limiting, the double-stranded nucleic acid comprises a sequence that is substantially complementary to SEQ ID NO:4, including without limitation, sequences that are 80%, 85%, 90%, 95 or 99% identical to SEQ ID NO: 4. In addition, the sequences may be defined by a range of identities of any two values or any values therein between. For example, but not wishing to be considered limiting in any manner, the present invention contemplates nucleic acids that are 78% identical or that are between 72% and 94% identical. Other identities and ranges of identities are also contemplated.

Also provided is a nucleic acid as defined above and throughout which is a gapmer. In a further embodiment, the gapmer is a methoxyethyl gapmer.

The nucleic acids described herein and throughout may be used in an assay to detect or identify a subject that has or is at risk of developing diabetes, obesity, inflammation or any combination thereof. In a preferred embodiment, the obesity is diet-induced obesity, the inflammation is adipocyte or liver tissue inflammation, or any combination thereof.

Also provided herein, the nucleic acids described above and throughout may be used for treating, reducing or preventing weight gain, obesity, diabetes or inflammation, or ameliorating a condition associated therewith in a subject in need thereof. Such conditions, may include but are not limited to one or more of diet-induced weight gain, diet-induced obesity, fat mass, liver inflammation, adipose tissue inflammation, adipose size, adipose macrophage accumulation, lipid accumulation, increasing the number of invariant natural killer T-cells (iNKT cells) in adipose tissue, improving glucose tolerance, insulin sensitivity, glucose homeostasis, fasted blood glucose, plasma insulin levels, response to both glucose and insulin challenge or any combination thereof.

In an embodiment of the present invention it is contemplated that the nucleic acids as described above and throughout are administered or for administration by visceral or subcutaneous injection into or adjacent to adipose tissue. They may be administered in any dosage regimen as necessary or desired, for example, but not limited to once a day, once a week or once a month.

Also provided herein is a vector comprising the nucleic acid as defined and throughout. Without wishing to be limiting, the vectors may be employed to express any of the nucleic acids described herein.

The present invention also provides an inhibitory agent that decreases the expression of a RIPK1 polypeptide, wherein said inhibitory agent is tozasertib, necrostatin 1, necrostatin 1s or GSK'547.

Further contemplated is a composition comprising the nucleic acid as described herein and throughout and a pharmaceutically acceptable carrier, diluent or excipient. In a further embodiment, the composition comprises a nucleic acid as described above or throughout and further comprises one or more primers that bind to the nucleotide sequence, a DNA polymerase capable of amplifying the nucleotide sequence with the primers, and optionally one or more restriction enzymes and/or buffers for carrying out amplification of the nucleotide sequence, restriction enzyme cleavage or a combination thereof.

Also provided herein is a primer set comprising a) a first primer that binds upstream (5′) of the polymorphic site on a nucleic acid that is defined above or herein and a second primer that binds downstream (3′) of the polymorphic site on a nucleic acid which is a complement thereto, for amplification of the nucleic acid sequence therein between, orb) a first primer that binds downtream (3′) of the polymorphic site on the nucleic acid that is defined above and a second primer that binds upsteam (5′) of the polymorphic site on a nucleic acid which is a complement thereto, for amplification of the nucleic acid sequence therein between. The composition may further comprise one or more buffers and optionally a polymerase for carrying out amplification reactions such as PCR.

Also provided by the present invention is a method of detecting or screening a subject for a RIPK1-associated nucleotide sequence or protein associated with weight gain, obesity, inflammation, diabetes, or a combination thereof, and optionally treating the subject, the method comprising identifying the presence or absence of the nucleotide sequence described herein in a biological sample from the subject, the sample comprising genomic DNA, mRNA or protein from the subject, wherein the presence of the nucleotide sequence is indicative that the subject has or is at risk for weight gain, obesity, inflammation, diabetes, or a combination or is a carrier for one or more genes associated weight gain, obesity, inflammation, diabetes, or a combination thereof, and optionally treating the subject. In a preferred embodiment, the one or more polymorphisms in the ripk1 gene are relative to:

a) rs67432438
(SEQ ID NO: 1)
CACTTTGTTGCCCAAGCTGGAGTGC[-/AGTG]AGTGGCATGCGATCTCG
GCTCACTG;
b) rs4959774
(SEQ ID NO: 2)
CTCCGGTGctctgtttctgtcccta[A/G]agttcttttcctttccacgg
agttt;
c) rs2272990
(SEQ ID NO: 3)
TGTGGAAACACAGAGACACCTTCCC[C/T]AAGCCTCCGCTGTCCAGTTC
TGCAC,
d) rs6907943
(SEQ ID NO: 4)
GTGTTTGTTTGCAGCTCGTTAGCAT[A/C]AAATTTGCAAATTGCTTGGT
AGTTT,
e) rs2064310
(SEQ ID NO: 5)
CTTGATCACCACCATACAGAAAGTA[A/C]CAGAAAAGATGTTTTCTCTT
CTTCC,
f) rs7753662
(SEQ ID NO: 6)
gtgtgatccaccatgcccagccTCA[T/G]CCTTCTTGAATAGCAATGAT
CACTC,
g) rs5873855
(SEQ ID NO: 7)
GTGAATTTAACTGCACTGGGTGCCT[-/TA]TATATAAGTGGAATCATAC
AGTATT,
h) rs141325626
(SEQ ID NO: 8)
TCCCCTCCCTTGTTTAGCTGGCATC[-/TTTAGAAAGTA]TTTTGCCGTC
TAGATTGGCCTTGTC,
or

the complement thereof, and wherein the polymorphic site is in brackets and wherein the risk alleles are defined by the polymorphisms described above.

The step of detecting as recited above may be performed by any method known in the art, for example, but not limited to microarray analysis, restriction analysis, probe hybridization, nucleotide sequence amplification, polymerase chain reaction (PCR), electrophoretic-based nucleic acid analysis, ELISA, DNA aptamers, molecular barcoding, DNA sequencing, protein sequencing, antibody binding analysis, mass spectrometry, gene chip analysis, or a combination thereof. In a preferred embodiment, the nucleic acid is genomic DNA or mRNA obtained from blood or adipose tissue.

In a further embodiment of the method, there may be one or more steps that precede the step of detecting, for example, but not limited to selecting a subject that is overweight, obese, or that exhibits one or more signs of diabetes or inflammation.

In a further embodiment of the method, there may be one or more steps that follow the method, for example, but not limited to administering an inhibitory agent or nucleic acid that reduces expression or activity of RIPK1 in the subject, changing the diet of the subject, increasing the exercise regimen of the subject, administration of one or more additional tests, administration of one or more surgical procedures, gene therapy, monitoring the subject, analyzing the body composition of the subject, counselling the subject, administration of additional therapeutic agents, stapling the stomach of the subject, applying a gastric band, gastric bypass, gastric balloon, and/or gastric sleeve to the subject.

In a further embodiment of the present invention, there is provided a method of treating a subject that exhibits weight gain, obesity, diabetes or inflammation. The subject may be known to comprise one or more polymorphisms as described herein. In an alternate embodiment the genotype of the subject may be unknown but exhibit one or more signs or symptoms associated with elevated levels of RIPK1 which are associated with weight gain, obesity, diabetes, inflammation or a combination thereof.

The present invention also contemplates a method as described above and throughout, in which the polymorphism is a mutation in a promotor sequence upstream of the RIPK1 gene.

In a further embodiment, there is also provided a method of identifying a polymorphism in a RIPK1 nucleic acid that is associated with risk of obesity, diabetes, inflammation or a combination thereof comprising:

detecting, in the nucleic acid, at least one polymorphism associated with increased expression of the RIPK1 human ripk1 gene;
wherein identification of the at least one polymorphism identifies the polymorphism in the RIPK1 nucleic acid as associated with risk of obesity, diabetes, inflammation or a combination thereof in a subject.
Also provided by the present invention is a compound which reduces expression of RIPK1. The compound may be used to reduce expression of RIPK1 in a cell in vitro or in a subject in vivo. Such compounds may be used for treating weight gain, obesity, diabetes or inflammation or ameliorating a condition associated therewith in a subject in need thereof. Representative conditions comprise, but are not limited to one or more of reducing liver inflammation, lipid accumulation, body weight, fat mass, increasing the number of invariant natural killer T-cells (iNKT cells) in adipose tissue, improving glucose tolerance, insulin sensitivity, glucose homeostasis, fasted blood glucose, plasma insulin levels, response to both glucose and insulin challenge or any combination thereof.

In a further embodiment, the present invention contemplates kits comprising one or more of:

a) one or more nucleic acid sequences defined above and throughout, wherein the nucleic acids are optionally labeled;
b) a RIPK1 polypeptide encoded by a nucleic acid that comprises the polymorphism defined above and throughout or a wild type variant thereof, or both;
c) a vector capable of expressing the one or more nucleic acids defined in a) or encoding the RIPK1 polypeptide of b);
d) an antibody that bind to the RIPK1 polypeptide of b);
e) a compound that reduces expression or activity of RIPK1;
f) one or more probes, primers, primer pairs as described herein;
g) one or more DNA aptamers that bind to RIPK1 polypeptide of b) or any one of SEQ ID NOs: 1-8;
h) a polymerase;
i) one or more buffers, restriction enzymes, dNTPs, microarrays, gene chips, assay plates, multi-well dishes, glass substrates, purification resins or beads or any combination thereof; wherein the nucleic acid, polypeptide, vector, composition or antibody is optionally physically associated or attached to the microarray, gene chip, assay plate, multi-well dish, glass substrate, purification resin or bead;
j) any combination of a-i).

In a further embodiment, there is provided a method for assessing risk, diagnosis, prognosis, or any combination, of obesity, diabetes, inflammation, or any combination, in a subject, comprising:

a) detecting, in a nucleic acid sample from said human subject, expression levels of RIPK1, E4BP4, or both; and
b) comparing expression level data obtained in step a) from said human subject to expression levels of either RIPK1, E4BP4, or both, from a healthy or affected control group to make a risk assessment, diagnosis, prognosis or any combination, of either obesity, diabetes, inflammation or any combination, in said human subject.

### BRIEF DESCRIPTION OF THE DRAWINGS

These and other features of the invention will become more apparent from the following description in which reference is made to the appended drawings wherein:

FIG. 1A is a regional association plot of SNPs at the RIPK1 locus with obesity.

FIG. 1B is an odds ratio for the index GWAS SNP rs6907943 (SEQ ID NO: 4) and LD SNPs, in the Ottawa OBLE cohort and comparison to rs9939609 (SEQ ID NO: 13) and LD SNPs in the FTO locus.

FIG. 1C is a plot depicting eQTL analysis of SNP, rs2064310 (SEQ ID NO: 5) in the RIPK1 locus and levels of RIPK1 mRNA in adipose tissue from the METSIM cohort.

FIGS. 1D-E are plots depicting RIPK1 mRNA expression in adipose tissue from humans categorized by BMI less than or greater than 25, and the correlation of relative RIPK1 mRNA expression and BMI. Data analyzed using a two-tailed Student's T-test *p<0.05 (FIG. 1D) and linear regression (FIG. 1E) (n=4 subjects BMI<25, n=6 subjects BMI>25).

FIG. 1F are scatter plots depicting Ripk1 mRNA expression in adipose tissue in mice from the Hybrid Mouse Diversity Panel and the correlation with fat mass measured by NMR and visceral fat mass at sacrifice in male mice.

FIG. 2A is a schematic model depicting how a candidate SNP regulates E4BP4 to affect RIPK1 expression.

FIG. 2B is a bar graph depicting human SW872 cells that were transfected with a plasmid containing the rs5873855 (SEQ ID NO: 7) sequence (either − or TA) plus the human RIPK1 promoter upstream of the firefly luciferase gene and a plasmid encoding human E4BP4. Luciferase activity was measured 24 h post-transfection. Data is mean±SEM of n=4 independent experiments, *p<0.05, two-tailed Student t-test.

FIG. 2C is a bar graph depicting data from a chromatin immunoprecipitation assay using antibodies against E4BP4 (or IgG as control) showing enrichment of the RIPK1 locus in SW872 cells. PPARγ, a known E4BP4 target gene, serves as a positive control. Data is mean±SD of n=3 independent experiments performed in technical duplicate,**p≤0.01, ***p≤0.001 vs. IgG control using one-way ANOVA.

FIG. 2D is a bar graph depicting siRNA knockdown of E4BP4 in SW872 liposarcoma cells and levels of RIPK1 mRNA expression, in untreated cells or cells treated with TNFα (20 ng/ml) for 24 h. Data is mean±SD of n=3 independent experiments performed in technical duplicate. *p<0.05, two-tailed Student's t-test.

FIG. 2E is a bar graph depicting analysis of E4bp4 and Ripk1 mRNA in NKT cells from adipose tissue or spleen. Expression is depicted as a ratio of gene expression in Spleen:Adipose. E4bp4 levels are high in adipose NKT and Ripk1 levels are low, whereas spleen NKTs have low expression of E4bp4 and high expression of Ripk1. Data analyzed from the GSE dataset GSE63358.

FIG. 2F is a bar graph depicting mRNA expression data from WAT from mice stimulated with vehicle or αGalCer. Data analyzed from the GSE dataset GSE36032 and expressed as log fold-change vs. vehicle control (n=3 vehicle, n=4 αGalCer, adjusted p-value corrected for multiple comparisons).

FIG. 3A is a plot of total body weights of male WT C57BL/6J mice (n=8/group) fed a high fat diet for 24 weeks while simultaneously administering 50 mg/kg control or RIP1-A or RIP1-B ASOs weekly.

FIG. 3B is a bar graph depicting fat, lean and total body mass measured using NMR (EchoMRI) of the test groups in FIG. 3A.

FIG. 3C is a bar graph depicting liver and epididymal adipose tissue weights at termination of study of the test groups in FIG. 3A.

FIGS. 3D and 3E are bar graphs depicting fasting blood glucose levels (FIG. 3D) and fasting blood insulin levels (FIG. 3E) of the test groups in FIG. 3A.

FIGS. 3F and 3G are plots depicting fasting glucose tolerance test results (GTT, FIG. 3F) or insulin tolerance tests (ITT, FIG. 3G) of the test groups in FIG. 3A.

FIGS. 3H and 3I are bar graphs depicting data obtained from mice in FIG. 3A caged in metabolic cages with indirect calorimetric and activity monitoring. VO2, respiratory exchange ratios (RER, FIG. 3H) and beam breaks (FIG. 3I) were recorded as a measure of physical activity.

FIG. 3J is a bar graph depicting total 48 h food consumption of the test groups in FIG. 3H. Data representative of mean±SEM of n=8 mice/group, *p<0.05, **p<0.01, ***p<0.001, ****p<0.0001

FIG. 4A is a bar graph depicting liver expression of Ripk1 mRNA after 24 weeks of ASO treatment and high fat diet.

FIG. 4B is a bar graph depicting mRNA expression of inflammatory genes in the liver.

FIG. 4C depicts oil-red tissue staining for neutral lipids in liver treated with control ASO (left) or RIP1 ASO-B (right).

FIG. 4D shows immunohistochemistry staining for the 2′F MOE moiety on the anti-sense oligonucleotides (ASO; left), RIPK1 (RIP1; right) in epididymal WT.

FIG. 4E is a bar graph depicting Ripk1 mRNA expression in subcutaneous and epididymal WAT in mice treated with control and RIP1 ASO.

FIG. 4F is a graph depicting adipocyte area in control ASO and RIPK1 ASO treated mice analyzed from H&E stained sections.

FIG. 4G is a set of images of Mac2 staining of epididymal WAT (left) and a bar graph depicting quantification of Mac2+ area (right).

FIGS. 4H and 4I are bar graphs depicting gene expression in epididymal WAT for pro-inflammatory genes Cd68 and Il 1a and M2 macrophage markers Fizz1 and Stat3 analyzed by qPCR (FIG. 4H, normalized to housekeeping genes) and Nanostring (FIG. 4I, normalized to housekeeping genes and expressed as counts). *p<0.05, **p<0.01 ****p<0.0001 by Student's t-test.

FIG. 5A are flow cytometry plots of adipose NKT cells identified as αGalCer-loaded CD1d-tetramer+CD3e+ (circled area).

FIG. 5B is a bar graph depicting NKT % (expressed as % of F4/80− cells that are αGalCer-loaded CD1d-tetramer+CD3e+) in adipose tissue from mice treated for 3 weeks with cont ASO or RIPK1 ASO (n=8/group).

FIG. 5C is are flow cytometry plots of NKT cells from matched spleen (αGalCer-loaded tetramer+CD3e+; circled area).

FIG. 5D is a bar graph depicting NKT (expressed as % of F4/80− cells that are αGalCer-loaded CD1d-tetramer+CD3e+) in spleen from mice treated for 3 weeks with cont ASO or RIPK1 ASO (n=8/group).

FIG. 5E is a bar graph depicting NKT % of T-cells from adipose tissue of cont ASO or RIPK1 ASO treated mice after 22 weeks high-fat diet.

FIG. 5F is a schematic representation of RIPK1 expression controlled by E4BP4, where active E4BP4 represses RIPK1 expression but promotes anti-inflammatory signaling, whereas SNP variants disrupt this repression and drive RIPK1 expression, NFκB activation and inflammation. # p<0.05 by Student's t-test.

FIG. 6 is a plot of body weight in grams over a 24 week period wherein treatment with a RIPK1 inhibitor included in feed in a diet-induced obesity (DIO) mouse model. Male WT C57BL/6J mice (n=8/group) were fed either a high fat diet (blue) or a high fat diet with a RIPK1 inhibitor compound included (red), for 18 weeks. After 18 weeks, the feed provided to the mice were switched between groups (Diet Switch, red arrow) until 24 weeks.

FIG. 7 shows Data from the Genotype-Tissue Expression (GTEx) database interrogating RIPK1 expression in visceral and subcutaneous adipose tissue. Number of subjects homozygous or heterozygous for the minor (Ref) or major (Alt) alleles for rs2064310 (SEQ ID NO: 5), rs2272990 (SEQ ID NO: 3) and rs67907943 (SEQ ID NO: 4) are indicated.

FIG. 8 shows four scatter plots depicting data from the Hybrid Mouse Diversity Panel (HMDP) showing correlations of Ripk1 mRNA expression and NMR and visceral fat mass at sacrifice (top two plots) and serum insulin and HOMA-IR index in males from approximately 100 inbred strains of mice (bottom two plots)¹⁹.

FIG. 9 is a series of bar graphs depicting serum cytokines from mice treated with cont ASO or RIPK1 ASO-B for 24 weeks. Cytokine analysis was done using the Bioplex Pro Cytokine 23-plex assay. No significant differences between cytokines IL-1α, TNFα, IL-4 or IL-10 were observed.

FIGS. 10A and 10B are bar graphs depicting flow cytometry analysis of % F480+ cells in stromal vascular fraction (SVF) of adipose tissue (FIG. 10A) or splenocytes from mice treated with control ASO or RIP1 ASO-B for 3 weeks while fed a high fat diet (FIG. 10B).

FIGS. 10C to H are bar graphs depicting flow cytometry analysis of circulating leukocytes from mice treated with control ASO or RIP1 ASO-B at 3 weeks (FIG. 10C-E) and 22 weeks (FIG. 10F-H) FIGS. 10C and 10F show data from Ly6Clow monocytes, FIGS. 10D and 10G show data from Ly6Chigh monocytes, and FIGS. 10E and 10H show data from CD3e+ T cells. *p<0.05 by Student's t-test.

FIG. 11 are flow cytometry plots depicting flow cytometry analysis of the isolated stromal vascular fraction (SVF) of adipose tissue from mice treated with control ASO (top) or RIP1 ASO-B (bottom) for 3 weeks while fed a high fat diet. Representative flow cytometry plots of unloaded-tetramer+CD3e+ (left) and αGalCer-loaded(CD1d)-tetramer+CD3e+ (right; circled area).

FIG. 12 depicts a representative gating strategy employed for flow cytometry analysis of F480+ macrophages and CD1d-tetramer+CD3e+iNKTs. Debris and doublets were excluded then F480+ cells were identified (F4/80+). For iNKTs, CD45+ cells were positively selected, from which CD8a+ and MHCII+ and then F4/80+ were excluded. iNKTs were identified as dually expressing CD3e+ and CD1d-tetramer+.

FIGS. 13A and 13B depict representative gating strategies employed for flow cytometry analysis of circulating myeloid panels. Debris and doublets were excluded then CD45+ cells were identified. From the CD45+ population, % of other populations were gated as displayed.

### DETAILED DESCRIPTION

One or more currently preferred embodiments have been described by way of example. It will be apparent to persons skilled in the art that a number of variations and modifications can be made without departing from the scope of the invention as defined herein and throughout.

RIPK1 (Receptor-Interacting serine/threonine Protein Kinase 1, SEQ ID NO: 9) is a central regulator of inflammatory cell function that coordinates inflammation, apoptosis and necroptosis in response to inflammatory stimuli. We show herein that genetic variants in the RIPK1 locus associate with obesity in humans and with higher expression levels of RIPK1 in adipose tissue, and discover E4BP4 as a novel transcriptional regulator of RIPK1 expression. Accordingly, blockade of Ripk1 in mice fed a high-fat diet reduces adiposity, adipose tissue inflammation, and improves insulin resistance. Together these data place RIPK1 at the nexus of metabolic inflammation and indicate that targeting of RIPK1 may be an effective way of combating inflammation in the setting of obesity and metabolic syndrome.

According to an embodiment of the present invention, which is not meant to be limiting, there is provided a nucleic acid associated with increased risk of weight gain, obesity, inflammation, diabetes or combination thereof, the nucleic acid comprising:

a) at least 7 consecutive nucleotides of SEQ ID NO: 1 and comprising “CAGTC” at position 26, or a sequence complementary thereto;
b) at least 7 consecutive nucleotides of SEQ ID NO: 2 and comprising G at position 27, or a nucleotide sequence that is complementary thereto;
c) at least 7 consecutive nucleotides of SEQ ID NO: 3 and comprising T at position 26, or a sequence complementary thereto;
d) at least 7 consecutive nucleotides of SEQ ID NO: 4 and comprising C at position 26, or a sequence complementary thereto;
e) at least 7 consecutive nucleotides of SEQ ID NO: 5 and comprising C at position 26, or a sequence complementary thereto;
f) at least 7 consecutive nucleotides of SEQ ID NO: 6 and comprising G at position 26, or a sequence complementary thereto;
g) at least 7 consecutive nucleotides of SEQ ID NO: 7 and comprising “TTA” at position 26, or a sequence complementary thereto;
h) at least 7 consecutive nucleotides of SEQ ID NO: 8 and further comprising TTTAGAAAGTA at position 26, or a sequence complementary thereto; or
i) at least 70% identity to the nucleotide sequence defined in any one of a)-h) and comprising the designated nucleotides noted above in a position relative thereto.
The nucleic acids may be useful, for example, but not limited to as probes to identify polymorphisms in nucleotide sequences, as primers for amplification reactions, as siRNA, antisense sequences or the like to reduce expression of RIPK1 in vitro or in vivo, for practicing methods to detect, diagnose, identify risk or treat conditions involving RIPK1-mediated disorders, including but not limited to diabetes, weight gain, obesity, inflammation, or any combination thereof.

According to an embodiment of the present invention, there is provided a method of assessing risk of obesity, diabetes, inflammation or a combination in a subject or sample. The method comprises genotyping a nucleic acid obtained from a subject to identify one or more polymorphisms associated with the effects noted above and may include detecting, in the nucleic acid, at least one polymorphism associated with human ripk1 gene. In some cases, the polymorphisms are upstream or downstream of the ripk1 gene. The one or more polymorphisms may be located within exons or outside of the coding sequence, such as within an intronic sequence, or a promotor. Examples of polymorphisms are described by SEQ ID NOS: 1-8 or complements thereof. The nucleic acid from the patient, subject or sample is preferably genomic DNA, more preferably a blood or adipose tissue sample. Identification of the at least one polymorphism identifies the patient or subject at risk of obesity, diabetes, inflammation or a combination thereof.

According to an embodiment of the present invention, there is provided a method of detecting or screening a subject for a nucleotide sequence or protein associated with weight gain, obesity, inflammation, diabetes, or a combination, the method comprising identifying the presence or absence the nucleotide sequence defined above in a biological sample from the subject, the sample comprising genomic DNA, mRNA or protein from the subject, wherein the presence of the nucleotide sequence is indicative that the subject has or is at risk for weight gain, obesity, inflammation, diabetes, or a combination or is a carrier for one or more genes associated weight gain, obesity, inflammation, diabetes, or a combination thereof.

According to an embodiment of the present invention, there is provided a method of genotyping a nucleic acid obtained from a subject to identify one or more polymorphisms and may include detecting with the use of at least one anti-sense oligonucleotide (ASO) complimentary to the at least one polymorphism. Other examples of detection methods or steps include but are not limited to: microarray analysis, restriction analysis, probe hybridization, nucleotide sequence amplification, PCR, electrophoretic-based nucleic acid analysis, ELISA, DNA aptamers, molecular barcoding, DNA sequencing, protein sequencing, antibody binding analysis, mass spectrometry, gene chip analysis, or any combination thereof.

According to an embodiment of the present invention, either before or after genotyping the subject or sample, one or more additional steps may be performed. Additional steps may include, but not limited to, administering an inhibitory agent that reduces RIPK1 expression in the sample or subject, changing the diet of the subject, increasing the exercise regimen of the subject, administration of one or more additional tests, for example, but not limited to blood tests, such as sugar levels, blood enzyme markers, metabolites or the like as would be understood by a person of skill in the art, analyzing the body composition of the subject, counselling the subject, administration of one or more additional therapeutic agents, surgical intervention, such as stomach resection, stapling or the like as would be understood by a person of skill in the art.

According to an embodiment of the present invention, there is provided a method of detecting a candidate subject for RIPK1 inhibitor treatment, said method comprising the steps of: a) detecting one or more polymorphisms of RIPK1 in a biological sample obtained from the candidate subject; b) determining RIPK1 expression levels; and c) correlating the RIPK1 expression levels in the biological sample to a control group which does not have obesity, diabetes, inflammation or a combination, wherein an elevated RIPK1 expression level in the test subject relative to the control group indicates that the subject is a candidate for RIPK1 inhibitor treatment.

According to an embodiment of the present invention, there is also provided a method of predicting a subject's risk to weight change, obesity, inflammation, diabetes, or a combination thereof, comprising: a) obtaining a biological sample comprising genomic DNA from the subject; b) determining the presence or absence of one or more polymorphisms in the ripk1 gene of the subject or the complement thereof, wherein the presence of said one or more polymorphisms is predictive of the subject's risk to weight change, obesity, inflammation, diabetes, or a combination thereof.

According to an embodiment of the present invention, there is provided a method for risk assessment, diagnosis, prognosis, or any combination, of obesity, diabetes, inflammation, or any combination, in a subject. The method may comprise a) detecting, in a nucleic acid sample from said human subject, expression levels of either RIPK1, E4BP4, or both; and b) comparing expression level data obtained in step a) from said human subject to expression levels of either RIPK1, E4BP4, or both, from either healthy or affected subjects, or both, to make a risk assessment, diagnosis, prognosis or any combination, of either obesity, diabetes, inflammation or any combination, in said human subject.

In one or more embodiments of the present invention, detection of expression levels of a repressor, such as E4BP4 is disclosed. In molecular genetics, a repressor is a DNA- or RNA-binding protein that inhibits the expression of one or more genes. Without wishing to be bound by theory or limiting in any manner, a DNA-binding repressor typically blocks the attachment of RNA polymerase to the promoter, thus preventing transcription of the genes into messenger RNA. Similarly, an RNA-binding repressor binds to the mRNA and prevents translation of the mRNA into protein. This blocking of expression is sometimes called repression. If an inducer, a molecule that initiates the gene expression, is present, then it can interact with the repressor protein and detach it from the operator. RNA polymerase then can transcribe the gene. A corepressor is a molecule that can bind to repressor and make it bind to the operator tightly, which decreases transcription. A repressor that binds with a corepressor is termed an aporepressor or inactive repressor. One or more SNPs, such as, but not limited to SEQ ID NO: 7, may act to disrupt the binding of repressors, such as E4BP4 and others, thereby increasing the transcription of the ripk1 gene.

As will be appreciated by a person skilled in the art, there are several well-known methods for detecting SNPs, all of which may be contemplated in the embodiments described herein. Non-limiting examples include known sequencing techniques which may or may not involve a PCR amplification step, microarray-based methods, exome sequencing methods, restriction enzyme-based methods, FISH-based methods, DASH-based methods, molecular beacon methods, and other methods known in the art.

The one or more polymorphic sites may be identified or isolated as polypeptides. As will be appreciated by a person skilled in the art, there are several well-known methods for detecting the presence of these mutant proteins and distinguishing them from wild-type protein. Non-limiting examples include mass-spectrometry-based methods, protein sequencing and antibody-based detection methods.

According to an aspect of the present invention, there is provided a method of amplifying, for example by PCR, a nucleic acid sequence containing CAGTC at position 26 of SEQ ID NO:1, G at position 27 of SEQ ID NO: 2, T at position 26 of SEQ ID NO: 3, C at position 26 of SEQ ID NOS: 4 and 5, G at position 26 of SEQ ID NO: 6, TTA at position 26 of SEQ ID NO: 7, or TTTAGAAAGTA at position 26 of SEQ ID NO: 8 using a first primer that binds upstream of said position and a second primer that binds downstream of said position and determining the genotype of the subject.

According to another aspect of the present invention, amplification of a nucleic acid sequence containing position CAGTC at position 26 of SEQ ID NO:1, G at position 27 of SEQ ID NO: 2, T at position 26 of SEQ ID NO: 3, C at position 26 of SEQ ID NOS: 4 and 5, G at position 26 of SEQ ID NO: 6, TTA at position 26 of SEQ ID NO: 7, or TTTAGAAAGTA at position 26 of SEQ ID NO: 8 may be achieved using any of the techniques known in the art, including but not limited to expansion from expression vectors or plasmids, and rolling circle replication-based amplification methods.

According to a further aspect of the present invention, there is provided a method substantially similar to the above described method, wherein the positions complementary to those noted above are interrogated.

According to an embodiment of the present invention, there is provided an amplification method substantially similar to that above, wherein a mRNA according to a complement of nucleic acid sequence of SEQ ID NOs: 1-8, 26-33 or fragment thereof is converted to a corresponding cDNA prior to further amplification.
According to a further aspect of the present invention, there is provided a method for identifying the presence of SNP mutations or insertions in nucleotide sequences such as in SEQ ID NOS: 1-8 by analyzing a sample using gel electrophoresis techniques well-known in the art to identify the presence or absence of CAGTC at position 26 of SEQ ID NO:1, G at position 27 of SEQ ID NO: 2, T at position 26 of SEQ ID NO: 3, C at position 26 of SEQ ID NOS: 4 and 5, G at position 26 of SEQ ID NO: 6, TTA at position 26 of SEQ ID NO: 7, or TTTAGAAAGTA at position 26 of SEQ ID NO: 8.

The sample obtained from a subject may comprise any biological sample from which genomic DNA may be isolated, for example, but not to be limited to a tissue sample, a sample of saliva, a cheek swab sample, blood, or other biological fluids that contain genomic DNA. In a preferred embodiment, which is not meant to be limiting in any manner, the sample is a blood sample. In another embodiment, RNA or mRNA is isolated from the subject.

Genotyping of SNPs as described herein may be performed by any method known in the art, for example, but not limited to PCR, sequencing, ligation chain reaction (LCR) or the like. In an embodiment, which is not meant to be limiting in any manner, amplifying the nucleic acid sequences containing the CAGTC at position 26 of SEQ ID NO:1, G at position 27 of SEQ ID NO: 2, T at position 26 of SEQ ID NO: 3, C at position 26 of SEQ ID NOS: 4 and 5, G at position 26 of SEQ ID NO: 6, TTA at position 26 of SEQ ID NO: 7, or TTTAGAAAGTA at position 26 of SEQ ID NO: 8 and genotyping the same is performed by PCR analysis using appropriate primers, probes and PCR conditions. Other methods known in the art may be used, for example molecular barcoding as used by NanoString Technologies in their nCounter™ platform.

In one embodiment, the step of amplifying the sequences containing the CAGTC at position 26 of SEQ ID NO:1, G at position 27 of SEQ ID NO: 2, T at position 26 of SEQ ID NO: 3, C at position 26 of SEQ ID NOS: 4 and 5, G at position 26 of SEQ ID NO: 6, TTA at position 26 of SEQ ID NO: 7, or TTTAGAAAGTA at position 26 of SEQ ID NO: 8 involves subjecting the nucleic acid sample to PCR, wherein the program for denaturing, annealing, amplifying is stored on a computer readable medium for execution by a microprocessor. The program causes a machine containing the samples to cycle through various temperatures for set periods of time. A similar or different machine comprising one or more programs may be employed to convert physical information, for example, but not limited to binding of nucleic acids or probes to target sequences, amplification or the like to a different state, such as electronic or otherwise, for example a signal that can be printed, displayed pictorially or digitized.

In a further embodiment, a restriction enzyme may be used to detect the presence of CAGTC at position 26 of SEQ ID NO:1, G at position 27 of SEQ ID NO: 2, T at position 26 of SEQ ID NO: 3, C at position 26 of SEQ ID NOS: 4 and 5, G at position 26 of SEQ ID NO: 6, TTA at position 26 of SEQ ID NO: 7, or TTTAGAAAGTA at position 26 of SEQ ID NO: 8 SNPs. One or more restriction enzymes may recognize the consensus sequence around one or both SNPs and/or insertions, and differentially cleave or not cleave wild-type versus mutant sequence. As such, a restriction enzyme recognizing all or a fragment of one or more nucleic acids with sequence according to SEQ ID Nos. 1-8, or the complementary wild-type or mutated sequence, can be used to easily determine the genotype of the subject. Other methods also may be used.

An apparatus, such as microarray or DNA chip, can be used to detect the presence or absence of CAGTC at position 26 of SEQ ID NO:1, G at position 27 of SEQ ID NO: 2, T at position 26 of SEQ ID NO: 3, C at position 26 of SEQ ID NOS: 4 and 5, G at position 26 of SEQ ID NO: 6, TTA at position 26 of SEQ ID NO: 7, or TTTAGAAAGTA at position 26 of SEQ ID NO: 8 SNPs or any other nucleic acid which results in a mutated DCPS protein as described herein. In this case, but without wishing to be limiting in any manner, an oligonucleotide may be bound to a substrate, which is suitable for this type of application. In an embodiment the oligonucleotide preferably comprises a contiguous nucleic acid, for example, the sequence from one or more of SEQ ID NOs. 1-8, 26-33 containing one or both SNPs described herein or a sequence substantially identical thereto. Another oligonucleotide can also be bound to the substrate. For example, but not wishing to be limiting, a nucleotide sequence comprising a complement of the nucleic acid sequences provided immediately above. In one embodiment the oligonucleotides are 7, 10, 12, 15, 16, 17, 19, 21, 23, 25 or more nucleotides in length. In another embodiment, the oligonucleotides are 60 nucleotides in length or more. Alternatively, the oligonucleotides may be defined by a range of any two of the values noted above or any two values therein between. A person skilled in the art will recognize that the length of the oligonucleotides can be altered based on the parameters of the assay. It is envisaged that the apparatus can contain other oligonucleotide sequences to confirm the subject's diagnosis or to test for the susceptibility of additional diseases or disorders, comorbid or otherwise.

While the present invention contemplates oligonucleotides comprising nucleotide sequences of at least about 7 nucleotides, preferably the nucleotide sequence comprises greater than 7 nucleotides, for example, but not limited to 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. Further the size of the oligonucleotides may be defined by a range of any two values as provided above or any two values in between. Also, the probe may be labeled by an appropriate moiety as would be known in the art, for example, but not limited to one or more fluorophores, radioactive groups, chemical substituents, enzymes, antibodies or the like to facilitate identification in hybridization assays and other assays or tests.

The nucleic acids as provided herein also may be employed to produce proteins which are associated with diabetes, obesity, inflammation as described herein.

While the present invention contemplates nucleic acids with at least 70% identity to those described above, the present invention also contemplates nucleotide sequences that hybridize to nucleotide sequences under stringent hybridization conditions. Stringent hybridization conditions may be, for example but not limited to hybridization overnight (from about 16-20 hours) hybridization in 4×SSC at 65° C., followed by washing in 0.1×SSC at 65° C. for an hour, or 2 washes in 0.1×SSC at 65° C. each for 20 or 30 minutes. Alternatively, an exemplary stringent hybridization condition could be overnight (16-20 hours) in 50% formamide, 4×SSC at 42° C., followed by washing in 0.1×SSC at 65° C. for an hour, or 2 washes in 0.1×SSC at 65° C. each for 20 or 30 minutes, or overnight (16-20 hours); or hybridization in Church aqueous phosphate buffer (7% SDS; 0.5M NaPO₄ buffer pH 7.2; 10 mM EDTA) at 65° C., with 2 washes either at 50° C. in 0.1×SSC, 0.1% SDS for 20 or 30 minutes each, or 2 washes at 65° C. in 2×SSC, 0.1% SDS for 20 or 30 minutes each for unique sequence regions.

The present invention is further directed to a nucleotide construct comprising the nucleic acid as described above operatively linked to one or more regulatory elements or regulatory regions. By “regulatory element” or “regulatory region”, it is meant a portion of nucleic acid typically, but not always, upstream of a gene, and may be comprised of either DNA or RNA, or both DNA and RNA. Regulatory elements may include those which are capable of mediating organ specificity, or controlling developmental or temporal gene activation. Furthermore, “regulatory element” includes promoter elements, core promoter elements, elements that are inducible in response to an external stimulus, elements that are activated constitutively, or elements that decrease or increase promoter activity such as negative regulatory elements or transcriptional enhancers, respectively. By a nucleotide sequence exhibiting regulatory element activity it is meant that the nucleotide sequence when operatively linked with a coding sequence of interest functions as a promoter, a core promoter, a constitutive regulatory element, a negative element or silencer (i.e. elements that decrease promoter activity), or a transcriptional or translational enhancer.

By “operatively linked” it is meant that the particular sequences, for example a regulatory element and a coding region of interest, interact either directly or indirectly to carry out an intended function, such as mediation or modulation of gene expression. The interaction of operatively linked sequences may, for example, be mediated by proteins that interact with the operatively linked sequences.

Regulatory elements as used herein, also includes elements that are active following transcription initiation or transcription, for example, regulatory elements that modulate gene expression such as translational and transcriptional enhancers, translational and transcriptional repressors, and mRNA stability or instability determinants. In the context of this disclosure, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns, or 3′ of the coding region sequence may also contribute to the regulation of expression of a coding region of interest. A regulatory element may also include those elements located downstream (3′) to the site of transcription initiation, or within transcribed regions, or both. In the context of the present invention a post-transcriptional regulatory element may include elements that are active following transcription initiation, for example translational and transcriptional enhancers, translational and transcriptional repressors, and mRNA stability determinants.

The regulatory elements, or fragments thereof, may be operatively associated (operatively linked) with heterologous regulatory elements or promoters in order to modulate the activity of the heterologous regulatory element. Such modulation includes enhancing or repressing transcriptional activity of the heterologous regulatory element, modulating post-transcriptional events, or both enhancing/repressing transcriptional activity of the heterologous regulatory element and modulating post-transcriptional events. For example, one or more regulatory elements, or fragments thereof, may be operatively associated with constitutive, inducible, tissue specific promoters or fragment thereof, or fragments of regulatory elements, for example, but not limited to TATA or GC sequences may be operatively associated with the regulatory elements of the present invention, to modulate the activity of such promoters within plant, insect, fungi, bacterial, yeast, or animal cells.

There are several types of regulatory elements, including those that are developmentally regulated, inducible and constitutive. A regulatory element that is developmentally regulated, or controls the differential expression of a gene under its control, is activated within certain organs or tissues of an organ at specific times during the development of that organ or tissue. However, some regulatory elements that are developmentally regulated may preferentially be active within certain organs or tissues at specific developmental stages, they may also be active in a developmentally regulated manner, or at a basal level in other organs or tissues within a plant as well.

By “promoter” it is meant the nucleotide sequences at the 5′ end of a coding region, or fragment thereof that contain all the signals essential for the initiation of transcription and for the regulation of the rate of transcription. There are generally two types of promoters, inducible and constitutive promoters.

An inducible promoter is a promoter that is capable of directly or indirectly activating transcription of one or more DNA sequences or genes in response to an inducer. In the absence of an inducer the DNA sequences or genes will not be transcribed. Typically the protein factor that binds specifically to an inducible promoter to activate transcription is present in an inactive form which is then directly or indirectly converted to the active form by the inducer. The inducer can be a chemical agent such as a protein, metabolite, growth regulator, or a physiological stress imposed directly by heat, cold, or toxic elements or indirectly through the action of a pathogen or disease agent such as a virus.

A constitutive promoter directs the expression of a gene throughout the various parts of an organism and/or continuously throughout development of an organism. Any suitable constitutive promoter may be used to drive the expression of the proteins or fragments thereof as described herein. Examples of known constitutive promoters include but are not limited to those associated with the CaMV 35S transcript. (Odell et al., 1985, Nature, 313: 810-812).

The term “constitutive” as used herein does not necessarily indicate that a gene is expressed at the same level in all cell types, but that the gene is expressed in a wide range of cell types, although some variation in abundance is often observed.

The gene construct of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3 prime end of the mRNA precursor.

The gene construct of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified so as to increase translation of the mRNA.

The present invention further includes vectors comprising the nucleic acids as described above. Suitable expression vectors for use with the nucleic acid sequences of the present invention include, but are not limited to, plasmids, phagemids, viral particles and vectors, phage and the like. For insect cells, baculovirus expression vectors are suitable. For plant cells, viral expression vectors (such as cauliflower mosaic virus and tobacco mosaic virus) and plasmid expression vectors (such as the Ti plasmid) are suitable. The entire expression vector, or a part thereof, can be integrated into the host cell genome.

Those skilled in the art will understand that a wide variety of expression systems can be used to produce the proteins or fragments thereof as defined herein. With respect to in vitro production, the precise host cell used is not critical to the invention. The proteins or fragments thereof can be produced in a prokaryotic host (e.g., E. coli or B. subtilis) or in a eukaryotic host (e.g., Saccharomyces or Pichia; mammalian cells, such as COS, NIH 3T3, CHO, BHK, 293, or HeLa cells; insect cells; or plant cells). The methods of transformation or transfection and the choice of expression vector will depend on the host system selected and can be readily determined by one skilled in the art. Transformation and transfection methods are described, for example, in Ausubel et al. (1994) Current Protocols in Molecular Biology, John Wiley & Sons, New York; and various expression vectors may be chosen from those provided, e.g., in Cloning Vectors: A Laboratory Manual (Pouwels et al., 1985, Supp. 1987) and by various commercial suppliers.

In addition, a host cell may be chosen which modulates the expression of the inserted sequences, or modifies/processes the gene product in a specific, desired fashion. Such modifications (e.g., glycosylation) and processing (e.g., cleavage) of protein products may be important for the activity of the protein. Different host cells have characteristic and specific mechanisms for the post-translational processing and modification of proteins and gene products. Appropriate cell lines or host systems can be chosen by one skilled in the art to ensure the correct modification and processing of the expressed protein.

The present invention also contemplates screening methods which identify and/or characterize the proteins as defined herein within biological samples from subjects. Such samples may or may not comprise DNA or RNA. For example, such screening or testing methods may employ immunological methods, for example, but not limited to antibody binding assays such as ELISAs or the like, protein sequencing, electrophoretic separations to identify the proteins as described above in a sample. As will be evident to a person of skill in the art, the screening methods allow for the differentiation of the proteins as defined herein from wild type proteins known in the art. Other methods include DNA aptamers, such as the SOMAscan™ and SOMAmer™ technology used by SomaLogic™ or molecular barcoding as used by NanoString Technologies in their nCounter™ platform. DNA aptamers may be screened for binding to a desired target, such as RIPK-1 oligonucleotide or polypeptide with desired SNPs, by a suitable method, such as systematic evolution of ligands by exponential enrichment (SELEX).

Also contemplated by the present invention is a nucleic acid comprising or consisting of a sequence selected from the group consisting of: a) a nucleic acid sequence comprising SEQ ID NOs. 1-8; b) a complement of a nucleic acid sequence comprising SEQ ID NOs. 1-8; c) a fragment of either a) or b); d) a nucleic acid sequence capable of hybridizing to any one of a), b) or c); and e) a nucleic acid sequence that exhibits greater than about 70% sequence identity with the nucleic acid defined in a), b) c) or d).

Also contemplated by the present invention is a nucleotide sequence associated with increased risk of weight gain, obesity, inflammation, diabetes or combination thereof, the nucleotide sequence comprising: a) at least 7 consecutive nucleotides of SEQ ID NO: 1 and comprising “CAGTC” at position 26, or a sequence complementary thereto; b) at least 7 consecutive nucleotides of SEQ ID NO: 2 and comprising G at position 27, or a nucleotide sequence that is complementary thereto; c) at least 7 consecutive nucleotides of SEQ ID NO: 3 and comprising T at position 26, or a sequence complementary thereto; d) at least 7 consecutive nucleotides of SEQ ID NO: 4 and comprising C at position 26, or a sequence complementary thereto; e) at least 7 consecutive nucleotides of SEQ ID NO: 5 and comprising C at position 26, or a sequence complementary thereto; f) at least 7 consecutive nucleotides of SEQ ID NO: 6 and comprising G at position 26, or a sequence complementary thereto; g) at least 7 consecutive nucleotides of SEQ ID NO: 7 and comprising “TTA” at position 26, or a sequence complementary thereto; h) at least 7 consecutive nucleotides of SEQ ID NO: 8 and further comprising TTTAGAAAGTA at position 26, or a sequence complementary thereto; or i) at least 80% identity to the nucleotide sequence defined in any one of a)-h).

A nucleic acid sequence exhibiting at least 70% identity thereto is understood to include sequences that exhibit 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9% or 100% identity, or a value therein between to SEQ ID NOs. 1-9. Further, the nucleic acid may be defined as comprising a range of sequence identities as defined by any two of the values listed or any values therein between.

Any method known in the art may be used for determining the degree of identity between nucleic acid sequences. For example, but without wishing to be limiting, a sequence search method such as BLAST (Basic Local Alignment Search Tool: (Altschul S F, Gish W, Miller W, Myers E W, Lipman D J (1990) J Mol Biol 215: 403-410) can be used according to default parameters as described by Tatiana et al., FEMS Microbiol Lett. 174:247-250 (1990), or on the National Center for Biotechnology Information web page at ncbi.nlm.gov/BLAST/, for searching closely related sequences. BLAST is widely used in routine sequence alignment; modified BLAST algorithms such as Gapped BLAST, which allows gaps (either insertions or deletions) to be introduced into alignments, PSI-BLAST, a sensitive search for sequence homologs (Altschul et al., (1997) Nucleic Acid Res. 25:3389-3402); or FASTA, which is available on the world wide web at ExPASy (EMBL-European Bioinformatics Institute). Similar methods known in the art may be employed to compare DNA or RNA sequences to determine the degree of sequence identity.

By way of example, a % identity value may be determined by the number of matching identical nucleotides or amino acids divided by the sequence length for which the percent identity is being reported. Percent (%) amino acid sequence similarity may be determined by the same calculation as used for determining % amino acid sequence identity, but may, for example, include conservative amino acid substitutions in addition to identical amino acids in the computation. Oligonucleotide alignment algorithms such as, for example, BLAST (GenBank; using default parameters) may be used to calculate sequence identity %.

An alternative indication that two nucleic acid sequences may be substantially identical is that the two sequences hybridize to each other under moderately stringent, or preferably stringent, conditions. Hybridization to filter-bound sequences under moderately stringent conditions may, for example, be performed according to Ausubel, et al. (eds), 1989, Current Protocols in Molecular Biology, Vol. 1, Green Publishing Associates, Inc., and John Wiley & Sons, Inc., New York, at p. 2.10.3. Alternatively, hybridization to filter-bound sequences under stringent conditions may, for example, be performed according to Ausubel, et al. (eds), 1989, supra. Hybridization conditions may be modified in accordance with known methods depending on the sequence of interest (see, for example, Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York. Generally, by way of non-limiting example, stringent conditions may be about 5° C. lower than the thermal melting point for the specific sequence at a defined ionic strength and pH.

Also contemplated by the present invention is a method comprising the steps of: isolating RNA from the subject; hybridizing an oligonucleotide comprising a contiguous nucleic acid capable of hybridizing to a nucleic acid of SEQ ID NOs. 1-9; wherein the presence of RNA complementary to the oligonucleotide is predictive of the presence or absence of the SNPs described herein.

The method of obtaining and analyzing DNA or RNA is not critical to the present invention and any method or methods may be used (e.g. Ausubel, et al. (eds), 1989, Current Protocols in Molecular Biology, Green Publishing Associates, Inc., and John Wiley & Sons, Inc., New York, at p. 2.10.3, or Maniatis et al., in Molecular Cloning (A Laboratory Manual), Cold Spring Harbor Laboratory, 1982, p. 387-389). For example, which is not to be considered limiting in any manner, DNA may be extracted using a non-enzymatic high-salt procedure (Lahiri and Nurnberger 1991). Alternatively, the DNA may be analyzed in situ. RNA can be isolated, for example, by phenol chloroform extraction and analyzed using RT-PCR.

It should be understood that following any method as described herein related to testing, identifying or screening, such methods may further comprise additional testing or screening for one or more additional genetic mutations, blood tests, blood enzyme tests, counseling, providing support resources or administering an additional pharmaceutical agent based on the results of such tests and/or screens. Similarly, it is further contemplated that such methods may be preceded by one or more steps, for example but not limited to selecting a subject that is obese or thought to be at risk of being obese, or selecting a subject that is diabetic or suspected of being at risk for diabetes.

It will be understood by those skilled in the art that a wide variety of methods and techniques known in the art may be used in carrying out certain embodiments of the present invention. By way of example, detection of the SNPs and other polymorphisms described herein may be accomplished using a variety of approaches and techniques well-known in the field, for example those described in U.S. Pat. No. 8,568,968 to Lenz and references cited therein, which are all incorporated by reference in their entirety. Lenz describes various conventional techniques in the art, including those described in:

Sambrook and Russell eds. MOLECULAR CLONING: A LABORATORY MANUAL, 3.sup.rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5.sup.th edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins-eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)) all of which are incorporated by reference.

Molecular beacons may be used to identify SNPs, as described, for example, by Lenz (U.S. Pat. No. 8,568,968), which further provides, for example, Tyagi and Kramer (1996) Nat. Biotechnol. 14:303-8; Kostrikis (1998) Science 279:1228-9; and Marras (1999) Genet. Anal. 14:151-6; Holland et al. (1991) Proc. Natl. Acad. Sci. 88:7276-7280; and U.S. Pat. No. 5,210,015 by Gelfand et al, all of which are incorporated by reference.

Mass spectrometry techniques may be used to identify SNPs as described herein, for example those described in Mass Spectrometry and Genomic Analysis, ed. Housby, 2001 which is incorporated by reference.

Microarrays may also be used in the detection of SNPs, for example those described in U.S. Pat. No. 8,568,968 to Lenz which include: Various “gene chips” or “microarray” and similar technologies are known in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarrying system that can dispense from 12 to 64 spots onto multiple glass slides (Intelligent Bio-Instruments); Molecular Biology Workstation and NanoChip (Nanogen, a microfluidic glass chip (Orchid Biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarray” are also described in US Patent Publ. Nos.: 2007-0111322, 2007-0099198, 2007-0084997, 2007-0059769 and 2007-0059765 and U.S. Pat. Nos. 7,138,506, 7,070,740, and 6,989,267 all of which are incorporated by reference.

The present invention may employ any of a wide variety of sequencing techniques useful in certain embodiments of the present invention. For example, U.S. Pat. No. 8,568,968 to Lenz provides examples of sequencing techniques and related methods which include: Maxam and Gilbert (1997) Proc. Natl. Acad. Sci. USA 74:560) or Sanger et al. (1977) Proc. Nat. Acad. Sci. 74:5463); Naeve et al. (1995) Biotechniques 19:448; U.S. Pat. No. 5,547,835 and International Patent Application Publication Number WO 94/16101, entitled DNA Sequencing by Mass Spectrometry by Koster; U.S. Pat. No. 5,547,835 and international patent application Publication Number WO 94/21822 entitled “DNA Sequencing by Mass Spectrometry Via Exonuclease Degradation” by Koster; U.S. Pat. No. 5,605,798 and International Patent Application No. PCT/US96/03651 entitled DNA Diagnostics Based on Mass Spectrometry by Koster; Cohen et al. (1996) Adv. Chromat. 36:127-162; and Griffin et al. (1993) Appl. Biochem. Bio. 38:147-159; U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In vitro DNA Sequencing”; U.S. Pat. No. 6,455,249; Cotton et al. (1988) Proc. Natl. Acad. Sci. USA 85:4397; Saleeba et al. (1992) Methods Enzy. 217:286-295 Myers et al. (1985) Science 230:1242); U.S. Pat. No. 6,455,249; Cotton et al. (1988) Proc. Natl. Acad. Sci. USA 85:4397; Saleeba et al. (1992) Methods Enzy. 217:286-295; Orita et al. (1989) Proc. Natl. Acad. Sci. USA 86:2766; Cotton (1993) Mutat. Res. 285:125-144 and Hayashi (1992) Genet. Anal. Tech. Appl. 9:73-79); Keen et al. (1991) Trends Genet. 7:5; Myers et al. (1985) Nature 313:495; Rosenbaum and Reissner (1987) Biophys. Chem. 265:1275; Saiki et al. (1986) Nature 324:163); Saiki et al. (1989) Proc. Natl. Acad. Sci. USA 86:6230 and Wallace et al. (1979) Nucl. Acids Res. 6:3543; Gibbs et al. (1989) Nucleic Acids Res. 17:2437-2448; U.S. Pat. No. 4,998,617 and in Landegren et al. (1988) Science 241:1077-1080. Nickerson et al. (1990) Proc. Natl. Acad. Sci, (U.S.A.) 87:8923-8927; U.S. Pat. No. 5,593,826. in To be et al. (1996) Nucleic Acids Res. 24:3728; U.S. Pat. No. 4,656,127; French Patent 2,650,840; PCT Publication No. WO 91/02087). As in the Mundy method of U.S. Pat. No. 4,656,127; Goelet et al. (PCT Publication No. 92/15712; Cohen et al. (French Patent 2,650,840; PCT Publication No. WO 91/02087; Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175).Syvanen et al. (1993) Amer. J. Hum. Genet. 52:46-59; U.S. Pat. No. 4,656,127; Cohen et al. (French Patent 2,650,840; PCT Publication No. WO 91/02087; U.S. Pat. No. 4,656,127, all of which are incorporated by reference.

Antibodies may be useful in some embodiments, the production and use of which are well known in the art, for example is described in Current Protocols in Immunology, Coico et al., John Wiley & Sons, which is hereby incorporated by reference.

In some aspects of the invention, RNA interference may be used. By way of non-limiting example, the present invention provides methods and uses for gene silencing nucleic acids (for example, but not limited to, siRNA, miRNA, shRNA, and others). In 2006 the Nobel Prize in Medicine was awarded for the discovery of RNA interference (RNAi). Early research showed that RNAi triggers, such as small interfering RNAs (siRNAs), could be readily designed to silence virtually any gene in a specific and potent manner. This suggested that gene silencing nucleic acids such as siRNAs could be used to treat a wide variety of diseases. Discovery of the antisense oligonucleotide (ASOs) and RNA interference (RNAi) gene silencing pathways provided researchers with tools for silencing the expression of genes of interest. These pathways are both triggered by the introduction of small nucleic acid molecules into cells. These small nucleic acid molecules are typically designed to be at least partially complementary to the mRNA transcribed from the gene or genes of interest, and recognition/binding of the mRNA by the small nucleic acid molecules (i.e. the gene silencing nucleic acids) generally triggers degradation of the mRNA through either steric blocking/prevention of translation, or enzymatic degradation or cleavage of the mRNA.

Generally, RNA interference is a mechanism whereby small approximately 21 double-stranded RNA molecules can potently silence or decrease expression of specific genes having complementary mRNA sequence. Organisms from plants to worms and humans have endogenous RNA silencing systems. For example, Argonaute (AGO) proteins bind small RNAs to silence gene expression. In humans, gene expression is reduced by cleaving and degrading RNA complementary to the gene silencing nucleic acid (i.e. siRNA guide strand), or repressing the translation of imperfectly complementary mRNA (such as in the case of miRNA gene silencing nucleic acids). In humans, the primary class of small RNA gene silencers are termed microRNAs (miRNA), which regulate large gene networks by repressing translation of mRNA with partially complementary binding sites (Fabian, 2010, Annu Rev Biochem, 79:351). miRNA are essential regulators of development, tumorigenesis and neurodegenerative disease (Pencheva, 2013, Nat Cell Biol, 15:546; Croce, 2009, Nat Rev Genet, 10:704; Abe, 2013, Trends Cell Biol, 23:30).

Complementary siRNAs elicit the enzymatic cleavage and degradation of target RNAs, eliciting a profound, rapid and specific silencing of a single gene. These complementary small RNAs, often called siRNAs or RNAi, are frequently used in research to study functions of specific genes and are in development for therapeutic treatment of patients. For instance, in pre-clinical studies a single dose of siRNA can eliminate >80% of gene expression for six weeks in the liver with only minor off-target effects (Coelho, 2013, N Engl J Med, 369:819; Kanasty, 2013, Nature Materials, 12:967). The ability to almost completely eliminate expression of a specific gene with a single siRNA, or a select group of genes with a pool of siRNA, has enormous therapeutic potential for many diseases starting with those caused by viruses or genetically mutated proteins that cause pathology. Near elimination of viral RNA, or cellular RNA before it produces disease-causing proteins, may represent a powerful therapeutic strategy for these diseases. RNAi may also be used to increase the efficacy of existing drugs by tailoring cellular responses or augmenting combinatorial effects on given pathways or physiological processes.

It will be understood that a gene silencing nucleic acid may be any nucleic acid which reduces, prevents, or silences the expression of a target gene. Without wishing to be limiting, suitable gene silencing nucleic acids may include siRNAs, antisense oligonucleotides (ASOs), short hairpin RNAs (shRNAs), microRNAs (miRNAs), or other RNA interference (RNAi) or antisense oligonucleotide (ASO) gene silencing triggers, among others. For example, a gene silencing nucleic acid may comprise an siRNA antisense strand, or an antisense oligonucleotide, which is fully, substantially, or partially complementary to a target mRNA. By way of non-limiting example, an siRNA/miRNA may be fully, substantially, or partially complementary (i.e. have seed-region complementarity at nucleotides 2-7) to a region of the gene-expressed mRNA sequence to be silencing by triggering RISC.

It will further be understood that a gene silencing nucleic acid may be a nucleic acid which affects transcription rates or epigenetic control of gene expression. Gene silencing nucleic acids may include, by way of non-limiting example, small RNAs with gene expression regulatory properties. By way of further non-limiting example, a gene silencing nucleic acid may comprise a CRISPR nucleic acid, such as a CRISPR guide RNA.

In an embodiment of the present invention, there is provided an inhibitory agent that reduces or down-regulates an amount of a RIPK1 polypeptide. In some cases, the inhibitory agent is an antisense oligonucleotide (ASO) comprising a nucleic acid sequence with a complementary region that is complementary to at least 5 contiguous nucleotides in SEQ ID NO: 1-9. The inhibitory agent may be an antisense oligonucleotide comprising a nucleic acid sequence partially identical, for example, 70%, 75%, 80%, 85%, 90%, 95% or 99% and others, to SEQ ID NOS: 1-9. The inhibitory agent may be, or is derived from, a miRNA, shRNA, Crispr guide RNA, or siRNA.

Complementary oligonucleotides or complementary regions within oligonucleotides may have at least 5 contiguous nucleotides with its binding region/oligonucleotide. In some cases, the complementary sequence is great than 5 nucleotides, for example 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, any number in-between, or more. Oligonucleotides may comprise more than one region of complementarity. Oligonucleotides may be single stranded and have two or more regions of complementarity that bind to one another.

The term “inhibitory agent” or “inhibitor of RIPK1” also may be used in this context to identify an entity, such as a small molecule which lowers or inhibits the activity of RIPK1, more particularly its enzymatic activity. A reduced activity of RIPK1 may be in reference to the activity of RIPK1 in a near-zero or zero concentration of the inhibitor, or in reference to the level of activity of RIPK1 at physiological conditions. In some cases, the inhibitory agent lowers the quantity of active RIPK1 in the cell. For example, the inhibitory agent may downregulate or silence transcription or translation of RIPK1. The inhibitory agent may prevent activation of RIPK1, for example by inhibiting a kinase that activates RIPK1 through phosphorylation. Reducing or inhibiting the activity of RIPK1 may refer to an in vitro context, such as a purified enzymatic assay or in a whole cell, or in vivo, such as a bacterial cell, a cell within a multicellular system or in a whole subject, such as a human. Examples of small molecule inhibitors include, but not limited to, tozasertib, necrostatins, GSK′547, GSK′481 and others. Necrostatins may represent a class of molecules, comprising suitable members such as necrostatin 1 and necrostatin 1s. Commercially available RIPK1 inhibitors may be used, such as GSK 2982772, GSK481, RIPA-56, and others.

When reviewing the various examples and/or embodiments outlined herein, the person of skill in the art will recognize that a gene silencing nucleic acid may be any nucleic acid which causes the expression of a particular gene within a cell to be reduced, prevented, or “silenced”. By way of non-limiting example, a gene silencing nucleic acid may be, or may be derived from, an siRNA (small interfering RNA), an antisense oligonucleotide (ASO), a short hairpin RNA (shRNA), a microRNA (miRNA), or another RNA interference (RNAi) or antisense gene silencing trigger, among others (see, for example, Gaynor et al., RNA interference: a chemist's perspective. Chem. Soc. Rev. (2010) 39, 4196-4184; Bennett et al., RNA Targeting Therapeutics: Molecular Mechanisms of Antisense Oligonucleotides as a Therapeutic Platform, Annual Review of Pharmacology and Toxicology, 50, 259-293). A gene silencing nucleic acid may decrease gene expression by any mechanism, for example but not limited to a pre- or post-transcriptional gene silencing technique as will be known in the art. Given a particular gene sequence, the person of skill in the art will be able to design gene silencing nucleic acids capable of targeting said gene sequence, reducing expression (either transcription, translation, or both) of the gene. Various software-based tools are available for designing siRNAs or ASOs for targeting a particular gene, including those available from the Whitehead Institute (http://sirna.wi.mit.edu/), or those available from commercial providers of siRNAs and ASOs. Gene silencing nucleic acids may be prepared as described in, for example, Current Protocols in Nucleic Acids Chemistry, published by Wiley.

It will be understood that, in certain non-limiting embodiments, a miRNA may include naturally expressed miRNA sequences, and also nucleic acids having a miRNA-like mechanism of action, but having a nucleic acid sequence which does not match a naturally expressed miRNA sequence.

It will also be understood that, in certain non-limiting embodiments, a nucleic acid as described herein may include one or more chemical modifications to the nucleic acid backbone, sugar, or nucleobase, as will be known to the person of skill in the art. By way of non-limiting example, a nucleic acid as described herein may be a modified nucleic acid comprising one or more chemical modifications which increase target binding affinity, specificity, stability, loading into Ago proteins, and/or resistance to nuclease degradation, and/or reduce off-target effects. Examples of chemical modifications to nucleic acids are well-known in the art, examples of which are described in, for example, Gaynor et al., RNA interference: a chemist's perspective. Chem. Soc. Rev. (2010) 39, 4196-4184 and Bennett et al., RNA Targeting Therapeutics: Molecular Mechanisms of Antisense Oligonucleotides as a Therapeutic Platform, Annual Review of Pharmacology and Toxicology, 50, 259-293.

In certain non-limiting embodiments, a gene silencing nucleic acid, such as a short hairpin RNA (shRNA)- or siRNA-type nucleic acid, or another miRNA or RNAi-type nucleic acid, or a suitable nucleic acid derived therefrom, may be used. The gene silencing nucleic acid may have full sequence complementarity to the mRNA or RNA target, partial sequence complementarity, or seed region complementarity. Sequences may have, by way of non-limiting example, 15nt-, 16nt-, 17nt-, 18nt-, 19nt-, 20nt-, 21nt-, 22nt-, 23nt-, or 24nt-sequence complementarity, either consecutively positioned or spread over the length of the nucleic acid sequence, or any range defined as spanning any two of these values, or any range defined as spanning any two of these values and excluding one or more of these values. By way of non-limiting example, a sequence may have full complementarity, such as 24nt full complementarity, or 17-19nt complementarity. In further non-limiting embodiments, sequence non-complementarity, or sequence mismatches, between the gene silencing nucleic acid, such as an shRNA or siRNA, and the mRNA or RNA target may occur at one or more sites, such as, for example, one or more of positions 3, 4, 5, 6, 7, 8, 9, 14, 15, 16, 17, 18, 19, 20, 21, 22, or 23, counting from the 5′ end of the mature gene silencing nucleic acid, or any range defined as spanning any two of these values, or any range defined as spanning any two of these values and excluding one or more of these values.

It will also be understood that a precursor of a gene silencing nucleic acid may be any nucleic acid sequence which is capable of providing a gene silencing nucleic acid to a cell. By way of non-limiting example, a pre-miRNA may be considered as a precursor of a gene silencing nucleic acid, as pre-miRNA is enzymatically processed by cells to produce mature miRNA. Similarly, longer double stranded DNA (dsRNAs) may be processed by cells to produce siRNAs. In certain non-limiting embodiments, a precursor of a gene silencing nucleic acid may be or may comprise a miRNA or siRNA incorporated within a nucleotide backbone sequence as described in further detail below, or an enzymatic cleavage product thereof.

Introduction of gene or a transcribed sequence into a cell may be accomplished using any of several methods known in the art. By way of example, a vector (either viral, plasmid, or other) comprising one or more copies of the particular gene each driven by a suitable promoter sequence (for example, a constitutive or inducible promoter), may be introduced into cells via transfection, electroporation, or viral infection, or another suitable method know in the art. Suitable expression vector techniques for overexpressing or introducing a particular gene into a cell are known in the art (see, for example, Molecular Cloning: A Laboratory Manual (4th Ed.), 2012, Cold Spring Harbor Laboratory Press). In some cases, genetic material, such as genes, transcribed material or ASOs, are delivered in the absence of any agent. For example, ASOs may be dissolved in saline and delivered subcutaneously.

Introduction of a gene (or a transcribed sequence/region), in the context of inserting a nucleic acid sequence into a cell, refers to “transfection”, “transformation”, or “transduction”, and includes the incorporation or introduction of a nucleic acid sequence into a eukaryotic cell where the nucleic acid sequence may optionally be incorporated into the genome of the cell, or transiently expressed (for example, transfected mRNA).

In some embodiments of the present invention, the inhibitory agent is a gapmer 80%, 85%, 90%, 95% or 99% identical to SEQ ID NOS: 1-9 or has a nucleic acid sequence with a complementary region that is complementary to at least 5 contiguous nucleotides in SEQ ID NO: 1-9. A gapmer may understood as a chimeric antisense oligonucleotide that contains a central block of deoxynucleotide monomers sufficiently long to induce RNase H cleavage. Gapmers may be designed synthetic oligonucleotides that are chemically modified. Chemical modifications may include: bridged nucleic acids (BNAs), methyl-O-ethyl (methoxyethyl, MOE), O-methyl, and others. Modifications may be made at the 2-prime position of the ribose or deoxyribose sugar. Other modifications may be made to the phosphodiester backbone of the oligonucleotide, such as a phosphorothioate, phosphodiamidate, and others. The sugar may be replaced with other ring structures such as a morpholine ring and others. The gapmer antisense oligonucleotides may act by recruiting RNase H to selectively cleave the targeted oligonucleotide strand. The cleavage of this strand initiates an antisense effect. This approach has proven to be a powerful method in the inhibition of gene functions and is emerging as a popular approach for antisense therapeutics. An example structure of a gapmer is BBBXXXXXXXBBB, where B comprises a modified nucleotide and X is a suitable number of unmodified or natural nucleotides.

A complementary region may be understood as a region of the oligonucleotide that is complementary to a sequence of interest. The complementary region may be a part of the sequence, such as 5 nucleotides or more, or the entire sequence.

The inhibitory agent may be a small molecule, such as a drug-like compound. In some cases, inhibition may be considered as downregulating the RIPK1 polypeptide, thereby reducing its overall activity. The inhibitory agent may bind to the RIPK1 polypeptide and at least partially inhibit or otherwise disrupt its activity. Inhibiting RIPK1 activity may result in reducing the ability of RIPK1 to act as a kinase and phosphorylate its substrate. Preferably, such inhibition is specific, i.e. the RIPK1 inhibitor reduces the ability of a RIPK1 polypeptide to phosphorylate its target at a concentration that is lower than the concentration of the inhibitor that is required to produce some other, unrelated biological effect.

The inhibitory agent may be used to treat a subject for a biological condition, such as obesity. In some cases, the RIPK1 inhibitory agent reduces liver inflammation in a subject, lipid accumulation, or both. The inhibitory agent may reduce body weight and fat mass in a subject. Body weight and fat mass may be reduced in subjects with a regular or abnormal diet, such as a calorie rich or high fat diet. Use of the inhibitory agent in a subject may increase the number of invariant natural killer T-cells (iNKT cells) in adipose tissue. iNKT cells may be increased in various tissues, such as liver tissue or any tissue where inflammation may occur.

Another non-limiting example of a treatable biological condition in a subject is diabetes. The inhibitory agent may improve glucose tolerance and insulin sensitivity. A person who is insulin-sensitive may need only a relatively small amount of insulin to keep blood glucose levels in the normal range. A person who is insulin-resistant, may need a lot more insulin to get the same blood-glucose-lowering effects. Insulin sensitivity may be improved in some cases by reducing macrophage count and promoting invariant natural killer T-cell accumulation in adipose tissue. The inhibitory agent may also improve glucose homeostasis, which may be understood as the balance of insulin and glucagon to maintain blood glucose. Improved homeostasis may also improve fasted blood glucose levels, plasma insulin levels and response to both glucose and insulin challenge in a patient.

Fasted blood glucose levels may be understood as the concentration of glucose in a subject's blood stream after a meal. Fasted blood glucose levels may be affected by the contents of the last meal, the size of the last meal and the body's ability to produce and respond to insulin.

As used herein, “insulin resistance” and “insulin sensitivity” refer to a physiological condition in which whole body or tissues including liver, skeletal muscle, adipose tissue fail or succeed, respectively, to response to insulin. As used herein, “type 2 diabetes” also refers to noninsulin-dependent diabetes mellitus (NIDDM) or adult-onset diabetes. It refers to a metabolic disorder caused by insufficient insulin production or insulin resistance which often manifested by a fasting glucose higher than 140 mg/dL. Insulin resistance may also be understood as a pathological condition in which cells fail to respond to insulin thus excess glucose in the blood stream cannot be removed into skeletal muscle or fat tissue.

A subject may be understood as a person or persons that is either healthy or obese. Obesity may be understood as an abnormal accumulation of fat such that health is impaired. Obesity may be measured by various methods, including but not limited to: body mass index (BMI), percent body fat (PBF), fat mass index (FMI), fat mass, lipid accumulation, body fat distribution, waist circumference (WC), waist-to-hip ratio (WHR), waist-to-height ratio (WHtR), waist-to-height ratio^{0.5} (WHtR^{0.5}), visceral adipose tissue (VAT) mass, adipose size, skinfold thickness measurements, bioelectrical impedance, underwater weighing, and dual energy x-ray absorptiometry (DXA). An obese person may be considered a human subject with a BMI at or above 30.0. Adipose tissue may be located at one or more locations on the subject, such as the hips, waist, legs and others.

Forms of administration include, but are not limited to, injections, solutions, creams, gels, implants, pumps, ointments, emulsions, suspensions, microspheres, particles, microparticles, nanoparticles, liposomes, pastes, patches, tablets, capsules, transdermal delivery devices, sprays, aerosols, or other means familiar to one of ordinary skill in the art. Pharmaceutical formulations of the present invention can be prepared by procedures known in the art using well-known and readily available ingredients. For example, the compounds can be formulated with common excipients, diluents, or carriers, and formed into tablets, capsules, suspensions, powders, and the like. Examples of excipients, diluents, and carriers that are suitable for such formulations include the following: fillers and extenders (e.g; starch, sugars, mannitol, and silicic derivatives); binding agents (e.g., carboxymethyl cellulose and other cellulose derivatives, alginates, gelatin, and polyvinyl-pyrrolidone); moisturizing agents (e.g., glycerol); disintegrating agents (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; coloring agents; perfuming agents; flavoring agents; lubricants (e.g., talc, calcium and magnesium stearate); solid polyethyl glycols; and mixtures thereof.

The formulations can be so constituted that they release the active ingredient only or preferably in a particular location, possibly over a period of time (i.e., a sustained-release formulation). Such combinations provide yet a further mechanism for controlling release kinetics. The coatings, envelopes, and protective matrices may be made, for example, from polymeric substances or waxes and the pharmaceutically acceptable carrier.

Pharmaceutically acceptable carriers include liquid carriers, solid carriers or both. Liquid carriers are aqueous carriers, non-aqueous carriers or both, and include, but are not limited to, aqueous suspensions, oil emulsions, water-in-oil emulsions, water-in-oil-in-water emulsions, site-specific emulsions, long-residence emulsions, sticky-emulsions, micro-emulsions and nano-emulsions. Solid carriers are biological carriers, chemical carriers or both and include, but are not limited to, viral vector systems, particles, microparticles, nanoparticles, microspheres, nanospheres, minipumps, bacterial cell wall extracts and biodegradable or non-biodegradable natural or synthetic polymers that allow for sustained release of the oligonucleotide compositions. Emulsions, minipumps and polymers can be implanted in the vicinity of where delivery is required (Brem et al. J. Neurosurg. 74:441, 1991). Methods used to a solid carrier, covalent coupling to the surface of the solid carrier, either directly or via a linking moiety, and covalent coupling to the polymer used to make the solid carrier. Optionally, mycobacterial cell wall-DNA complexes can be formulated by adding non-ionic or ionic polymers such as polyoxyethylenesorbitan monooleates (TWEENs), chitosan, chemically modified chitosan, hyaluronic acid, sodium hyaluronate salts, chondroitin sulphate, heparin, heparin sulphate or chemical modifications of these molecules. The molecular weight range of such polymers can range from less than 100 Da to greater than 5 million Da depending on the degree of polymerization and chemical modification therein.

Preferred aqueous carriers include, but are not limited to, water, saline and pharmaceutically acceptable buffers. Preferred non-aqueous carriers include, but are not limited to, a mineral oil or a neutral oil including, but not limited to, a diglyceride, a triglyceride, a phospholipid, a lipid, an oil and mixtures thereof, wherein the oil contains an appropriate mix of polyunsaturated and saturated fatty acids. Examples include, but are not limited to, squalane, squalene, soybean oil, canola oil, palm oil, olive oil and myglyol, wherein the fatty acids can be saturated or unsaturated. Optionally, excipients may be included regardless of the pharmaceutically acceptable carrier. These excipients include, but are not limited to, anti-oxidants, buffers, and bacteriostats, and may include suspending agents and thickening agents.

Methods of administration of compositions comprising probes and/or inhibitory agents of RIPK1 and other materials such as carriers of the present invention that are particularly suitable for various forms include, but are not limited to the following types of administration, oral (e.g. buccal or sublingual), anal, rectal, as a suppository, intracolonic, topical, parenteral, nasal, aerosol, inhalation, intrathecal, intraperitoneal, intravenous, intra-arterial, transdermal, intradermal, subdermal, subcutaneous, intramuscular, intralymphatic, intrauterine, intravesicular, vaginal, visceral, into a body cavity, surgical administration at the location of the inflamed tissue such as adipose tissue, into the lumen or parenchyma of an organ, into bone marrow and into any mucosal surface of the gastrointestinal, reproductive, urinary and genitourinary system. It is to be understood that the choice of route of administration of probes and/or inhibitory agents of RIPK1 will be selected by one of ordinary skill in the art of treatment such that RIPK1 inhibition or reduction in expression levels is achieved.

The compositions of the present invention can be applied in the form of creams, gels, solutions, suspensions, liposomes, particles, or other means known to one of ordinary skill in the art of formulation and delivery of the compositions. Ultrafine particle sizes can be used for inhalation delivery of therapeutics. Some examples of appropriate formulations for subcutaneous administration include, but are not limited to, implants, depot, needles, capsules, and osmotic pumps. Some examples of appropriate formulations for vaginal administration include but are not limited to creams and rings. Some examples of appropriate formulations for oral administration include but are not limited to: pills, liquids, syrups, and suspensions. Some examples of appropriate formulations for transdermal administration include but are not limited to gels, creams, pastes, patches, sprays, and gels. Formulations suitable for parenteral administration include, but are not limited to, aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. Extemporaneous injection solutions and suspensions may be prepared from sterile powders, granules and tablets commonly used by one of ordinary skill in the art.

Embodiments in which the compositions of the invention are combined with, for example, one or more pharmaceutically acceptable carriers or excipients may be prepared by conventional pharmaceutical techniques. Such techniques include the step of bringing into association the compositions containing the active ingredient and the pharmaceutical carrier(s) or excipient(s). In general, the formulations are prepared by uniformly and intimately bringing into association the active ingredient with liquid carriers. Preferred unit dosage formulations are those containing a dose or unit, or an appropriate fraction thereof, of the administered ingredient. It should be understood that in addition to the ingredients particularly mentioned above, formulations comprising the compositions of the present invention may include other agents commonly used by one of ordinary skill in the art.

The present invention also contemplates products and kits for practicing the methods of the present invention. For example, a kit may comprise:

a) one or more primers to amplify a nucleotide sequence that comprises the polymorphism as defined in any one of SEQ ID NOs:1-8;
b) one or more probes that hybridize to SEQ ID NOs:1-8, over a region of nucleotides comprising the polymorphic site, wherein said probe hybridizes to a particular variant of the polymorphism shown at the polymorphic site. Without wishing to be limiting in any manner, the probes may be labeled with an appropriate group, for example, a fluorescent tag, fluorophore, radioactive label or the like. Further, the one or more probes may be attached covalently or physically associated with a support for example, but not limited to a biochip, array, slide, multiwell plate, bead or the like. In an embodiment, which is not meant to be limiting in any manner, the probes may comprise an array of nucleic acids;
c) one or more reagents and/or products including, but not limited to, one or more buffers for performing PCR or probe hybridization, or any step in such a process as would be known to a person of skill in the art, one or more DNA amplifying enzymes, or any combination thereof;
d) one or more reagents, components and products for genotyping the polymorphisms as described herein, including, but not limited to those used in exonuclease assays, nucleotide sequencing, or any combination thereof;
e) one or more reagents, components or products for performing a DNA sequencing reaction that determines the sequence of a nucleotide sequence comprising SEQ ID NOs:1-8;
f) a gene chip or array comprising one or a plurality of nucleotide sequences comprising or consisting of SEQ ID NOs:1-8, and;
g) one or more sets of instructions for using the components as described herein, practicing the methods of the present invention as described herein, interpreting the data obtained from practicing the methods of the present invention or;
h) any combination or sub-combination thereof.

Also provided by the present invention are individual components of the kit, for example, but not limited to any product, composition described in the kit or elsewhere in the application. In a representative embodiment, the present invention provides one or more nucleic acid primers or probes.

The kit may include: one or more buffers, primers, restriction enzymes, dNTPs, microarrays, gene chips, assay plates, multi-well dishes, glass substrates, purification resins or beads or any combination thereof, wherein the nucleotide sequence, polypeptide, vector, composition or antibody is optionally physically associated or attached to the buffer, primer, restriction enzyme, dNTP, microarray, gene chip, assay plate, multi-well dish, glass substrate, purification resin or bead or one or more DNA aptamers that bind to RIPK1 polypeptide or any one of SEQ ID NOs: 1-8.

The nucleic acid primers and probes may be of any suitable length for use in the method of the present invention. Without wishing to be limiting in any manner, it is generally preferred that the primers and probes be between about 9 and about 100 nucleotides, for example, but not limited to about 7, 8, 9, 10, 11, 12, 13, 14, 15, 16, 17, 18, 19, 20, 21, 23, 25, 27, 29, 30, 35, 40, 45, 50, 60, 70, 80, 90, about 100 nucleotides or any amount therein between. The length of the primers and probes may also be defined by a range of any two of the values provided above or any two values therein between. With respect to probes, it is generally preferred that the probe comprise at least one, more preferably 3 or more nucleotides on each side of the polymorphic site. It is also contemplated that one or more of the primers or nucleic acid probes may be labeled as is known in the art, for example, but not limited to, with a radioactive element or tag, fluorophore, or the like.

Also provided by the present invention is a microarray, gene chip or the like which comprises the nucleotide sequence defined by one or more of SEQ ID NOs: 1-8 or a fragment thereof which comprises the polymorphic site. Preferably the microarray or gene chip comprises nucleotide sequences defined by one or more of SEQ ID NOs: 1-8. The microarray also may comprise the complement of the nucleotide sequences or a fragment thereof which comprises the polymorphic site. Preferably, the nucleotide sequences are of a length such as, but not limited to 7, 8, 9, 10, 11, 12, 13, 14, 15, 16, 17, 18, 19, 20, 21, 22, 23, 24, 25 or more continuous nucleotides to permit strong hybridization under stringent hybridization conditions. In a preferred embodiment the microarray comprises or consists of one or more nucleotide sequences comprising the polymorphic sites in SEQ ID NOs: 1-8 as described herein. However, the microarray may comprise additional nucleotide sequences for other genes, for example, but not limited to those involved or implicated in the diagnosis or development of obesity, diabetes, inflammation or the like.

The person of skill in the art will understand that biomolecules and/or compounds described herein may be provided in pharmaceutical compositions together with a pharmaceutically acceptable diluent, carrier, or excipient, and/or together with one or more separate active agents or drugs as part of a pharmaceutical combination or pharmaceutical composition. In certain embodiments, the biomolecules, compounds, and/or pharmaceutical compositions may be administered in a treatment regimen simultaneously, sequentially, or in combination with other drugs or pharmaceutical compositions, either separately or as a combined formulation or combination.

Biomolecules, compounds, and/or compositions as described herein may include one or more pharmaceutically acceptable excipients, diluents, and/or carriers. A pharmaceutically acceptable carrier, diluent, or excipient may include any suitable carrier, diluent, or excipient known to the person of skill in the art. Examples of pharmaceutically acceptable excipients may include, but are not limited to, cellulose derivatives, sucrose, and starch. The person of skill in the art will recognize that pharmaceutically acceptable excipients may include suitable fillers, binders, lubricants, buffers, glidants, and disentegrants known in the art (see, for example, Remington: The Science and Practice of Pharmacy (2006)). Examples of pharmaceutically acceptable carriers, diluents, and excipients may be found in, for example, Remington's Pharmaceutical Sciences (2000—20th edition) and in the United States Pharmacopeia: The National Formulary (USP 24 NF19) published in 1999.

The present invention will be further illustrated in the following examples.

### EXAMPLES

Methods

RIPK1 SNP Association with Obesity

Human subjects in the OBLE study were participants of either the Thin Gene Study (lean subjects) or well matched obese subjects recruited through the Ottawa Hospital's Weight Management Clinic and approved by the Ottawa Health Science Network Research Ethics Board¹⁴. Subjects with medical conditions affecting weight gain or with non-Caucasian ancestry were excluded. Those with BMI (Body Mass Index) greater than 32 kg/m² were categorized as obese and those with BMI less than 23 kg/m² were categorized as lean subjects. Tests of association were performed using the logistic model implemented in PLINK toolset (v1.07)¹⁵ and specifying sex and age and first two principal components of ancestry as covariates.

eQTL Analysis

RIPK1 expression quantitative trait loci (eQTL) were calculated as described previously.¹⁷ We applied the probabilistic estimation of expression residuals (PEER) method to infer and account for complex non-genetic factors affecting gene expression levels. This method is designed to detect the maximum number of cis-eQTLs. To optimize the discovery of trans-eQTLs within the same analysis, we performed PEER analysis by examining 10-50 inferred factors (Nk) at increments of five factors. We then used Matrix-eQTL to assess the genetic association with inverse normal-transformed PEER-processed residuals from RMA-normalized expression data. We examined variants to determine the number of trans-eQTL target genes by using various numbers of PEER factors. We selected Nk=35 as a single analysis to maximize the number of target genes at this locus; this threshold captured the 94.8% of cis-eQTLs identified with 50 PEER factors. For downstream eQTL mapping, we used the inverse normal-transformed PEER-processed residuals after accounting for 35 factors. Correlations were calculated with the biweight midcorrelation method (bicor) as implemented in the WGCNA R package.

Human Adipose Tissue Expression Analysis

All human tissue was collected according to approved IRB protocol at New York University Medical Center. Human fat tissue samples were homogenized in TRIzol reagent (Ambion, Life Technologies) and RNA was purified using the Direct-zol RNA MiniPrep Kit (Zymo Research) as described previously⁴². Extracted mRNA was then reverse transcribed using the iScript cDNA Synthesis Kit (Bio-Rad). Quantitative real-time PCR was performed with KAPA SYBR FAST qPCR Kits (KAPA Biosystems) on a QuantStudio 3 (Applied Biosystems) in triplicates. Results were expressed as normalized fold change calculated by the comparative cycle method (2^{-ΔΔCt}). Primer sequences used were as follows:

(SEQ ID NO: 14)
28S: F TGGGAATGCAGCCCAAG,
(SEQ ID NO: 15)
R CCTTACGGTACTTGTTGACTATCG,
(SEQ ID NO 16)
RIPK1: F AGACTGCTCGTCAAGTGTGG,
(SEQ ID NO: 17)
R TTGAGCTGTAGCCTGAACCTTA,

Cloning of the RIPK1 Promoter and Rs5873855 (SEQ ID NOs:7, 32) Regulatory Sequences

We first inserted the RIPK1 promoter sequence into the multiple cloning site of the pGL3-Basic vector (Promega) by digesting pGL3-Basic with MluI and HindIII. The RIPK1 genomic promoter sequence was PCR amplified using primers with MluI and HindIII restriction sites (see Table 3 below). The amplified promoter sequence was ligated with digested pGL3-Basic using T4 DNA Ligase (New England Biolabs). Next, a 309 bp intronic sequence containing the rs5873855 SNP (SEQ ID NO: 7) was inserted downstream of the luciferase gene in pGL3-Basic using Gibson assembly (New England Biolabs). pGL3-Basic-RIPK1 Promoter was digested with the SalI to linearize the plasmid, run on an agarose gel and gel purified with the Geneaid GenepHlow Kit (Frogga Biosciences). The rs5873855 genomic sequence and linearized pGL3-Basic-RIPK1 Promoter were assembled using the Gibson Assembly Cloning Kit (New England Biolabs).

List of primer sequences for cloning

| | Primer | |
|---|---|---|
| Region | direction | Sequence (5′-3′) |
| RIPK1 | FWD | GATTACGCGTTGCAGTTCCAACTTTGCTC |
| promoter | | (SEQ ID No: 18) |
| | REV | GATTAAGCTTTGGCTTCTTCAAGTGTAAG |
| | | (SEQ ID NO: 19) |
| rs5873855 | FWD | gtaaaatcgataaggatccgGGCAGTAAG |
| Element | | TACAGTCCC (SEQ ID NO: 20) |
| | REV | aaggctctcaagggcatcggTCAAAGCCA |
| | | CCTAAGTGC (SEQ ID NO: 21) |
| rs5873855 | FWD | taTATATAAGTGGAATCATACAGTATTTG |
| SDM | | TCC (SEQ ID NO: 22) |
| | REV | AGGCACCCAGTGCAGTTA (SEQ ID |
| | | NO: 23) |

RIPK1 Dual Luciferase Assays

SW872 cells were grown in 24 well plates until reaching 40-50% confluence. Each well was transfected with 250 ng of firefly luciferase reporter DNA (containing the intronic region of RIPK1, as described above), 10 ng of pRL-TK Renilla reporter (Promega), and co-transfected with 250 ng E4BP4 expression plasmid (OriGene, in pCMV6-XL5 backbone). All transfections were carried out using Lipofectamine 3000 reagent (Thermo Fisher Scientific). SW872 cells were harvested with 100 μL of 1× Passive Lysis Buffer per well 24 hours after transfection. Dual luciferase assays were conducted using 20 μL of lysate, the Dual-Luciferase Reporter Assay Kit (Promega), and the GloMax luminometer (Promega) as per manufacturer's instructions.

Chromatin Immunoprecipitation

SW872 cell genomic DNA was first genotyped for the rs5873855 (SEQ ID NO: 7) variant (homozygous for no insertion) via Sanger sequencing. SW872 cells were seeded in 10 cm dishes and grown to confluence before harvesting. Cells were fixed with 1% paraformaldehyde for 10 minutes before being quenched with glycine. Cells from two 10 cm dishes were washed twice with cold 1×PBS, pooled after scraping, spun for 10 minutes at 1000 g at 4° C., and pellets lysed with 500 μL of nuclear lysis buffer (50 mM Tris-HCl pH 8, 150 mM NaCl, 1 mM EDTA pH 8, 0.1% Triton X-100, 0.1% Sodium Deoxycholate, 0.1% SDS, protease inhibitors). Cross-linked chromatin was sheared into fragments of approximately 100-500 bp using the BioRuptor sonicator (Diagenode) and clarified using centrifugation. PureProteome Protein A/G Magnetic Beads (Millipore) were pre-blocked with sonicated salmon sperm DNA plus BSA for 30 minutes at 4° C. Before the immunoprecipitation, the 500 μL of total chromatin was incubated with resuspended pre-blocked beads to further reduce background. Each chromatin immunoprecipitation reaction used 200 μg of SW872 chromatin and 10 μg of either NFIL3 antibody cocktail (5 μg sc-74415X, 5 μg sc-374451X, Santa Cruz Biotechnology) or 10 μg of nonspecific mouse IgG (Abcam ab37355). SW872 chromatin was first incubated with antibody for 1 hour at 4° C. with rotation (chromatin samples were diluted to 1 mL). Next, 10 μL of resuspended pre-blocked beads were added to each tube and protein-antibody-bead complexes were incubated overnight at 4° C. with rotation. Complexes were washed four times with ChIP wash buffer, once with high salt wash buffer, and eluted from the beads with elution buffer containing 1% SDS and 100 mM sodium bicarbonate and heating for 15 minutes at 65° C. Cross-links were reversed by addition of NaCl to a final concentration of 200 mM along with RNase A and shaking at 65° C. for 5 hours. Eluted DNA was purified using the GenepHlow Gel/PCR Kit (Geneaid), and used for quantitative PCR. Quantitative PCR was performed using SYBR Green I Master (Roche) and the LightCycler 480 II machine (Roche). The sequences of primers specific for the rs5873855 DNA (SEQ ID NO: 7) sequence were FWD: 5′-GTCCCATAGTACATTCACTTTCC-3′ (SEQ ID NO: 24) and REV: 5′-AAGCCAGACACAAAAGGACAA-3′ (SEQ ID NO: 25).

Bioinformatic Analysis

Affymetrix MoGene 1.1ST CEL files were downloaded from the GEO GSE36032 and GSE63358 accession and RMA normalized using the R Bioconductor “oligo” library⁴³. Fold change analysis was performed using the “limma” library⁴⁴, with adjusted p-values generated by the FDR method. Gene Symbol annotations were assigned to transcript cluster identifiers using the “mogene1 1sttranscriptcluster.db” library (MacDonald J W (2017). R package version 8.7.0)

Animals

All animal use was approved by the University of Ottawa Animal Care Committee according to the Canada Council on Animal Care. Eight-week old male C57BL/6J mice were purchased from Charles River and RIPK1^{K45A} and their corresponding wild-type littermates were a generous gift from Dr Peter Gough (GlaxoSmithKline)²⁶. All mice (males) were fed a high-fat diet containing 60% kcal from fat (D12492, Research Diets Inc). C57Bl/6J mice (8 mice/group) were subjected to weekly subcutaneous injections of saline or control anti-sense oligonucleotides (ASOs) or 2 independent targeting RIP1 ASOs [methoxyethyl (MOE) gapmers obtained from Ionis Pharmaceuticals, USA] for 24 weeks. Body weight was measured weekly throughout the study and food consumption was measured weekly for two weeks. At sacrifice, mice were fasted for 6 hrs and anaesthetized with isofluorane prior to cardiac puncture, exsanguination and perfusion with PBS as previously described⁴⁵. Tissues were collected, snap-frozen and stored at −80.

Glucose and Insulin Tolerance Tests

As described previously⁴⁵, mice were fasted for 6 h and then subjected to intra-peritoneal injections of 1 g/kg of D-glucose (Fisher Scientific) or 0.75 units/kg of insulin (HI0213, Eli Lilly Canada Inc). Blood glucose was measured from the tail vein at depicted time intervals with an Accu-Check Aviva Nano glucose meter (Roche Diagnostics). Blood glucose levels in each group were averaged and plotted for each time point and area under of the curve was determined for each individual mouse and the mean±SEM for each group is represented within the GTT/ITT graph.

Metabolic Outputs and Energy Expenditure

Mice were placed individually in a customized 12-chamber Oxymax open-circuit indirect calorimeter equipped with laser beam sets (Columbus Instruments, Columbus, Ohio) and allowed to acclimatize for 48 hours and data were collected for another 48h period. The 2.5-L plexiglass chambers were supplied with air at 0.5 L/min, and were maintained at 28° C. throughout a 12 h light/dark cycle. In each chamber, concentrations of O₂ and CO₂ in dry air were measured for 60 seconds every 8-12 mins. Whole body O₂ consumption (VO₂) corrected for lean body mass (EchoMRI, Houston Tex.) were collected and respiratory exchange ratios (RER) were calculated as previously described⁴⁵.

RNA Isolation, Quantitative Real-Time PCR and Nanostring Analysis

Tissues were placed in Trizol reagent (Invitrogen) and homogenized using the Bullet Blender homogenizer (NextAdvance Inc). Total RNA was isolated and cDNA was synthesized using iScript Reverse Transcription kit (Biorad) as per manufacturer's instructions. Quantitative real-time PCR was performed in triplicate using either Evagreen Express QPCR Master Mix (ABM, Canada) or Taqman Gene Expression Assays as per manufacturer's instructions and mRNA level of target genes were normalized to an average of at least 2 housekeeping genes (B2m, Hprt, Sdha). For some genes (i.e. Fizz1, Stat3, Ripk1), NanoString nCounter Mouse Inflammatory CodeSet was used and average counts compared using nSolver Analysis software after normalization to housekeeping genes and internal controls as per the manufacturer's instructions.

Immunohistochemistry and Oil Red O Staining

10 mm thick formalin-fixed frozen liver and adipose tissue sections were stained with primary antibodies (anti-ASO, gift from Ionis Pharmaceuticals, 1:10000; anti-RIP1, Life Techonologies PA5-20811, 1:40; anti-Mac-2, Cedarlane CL8942AP, 1:500) at 4 degrees overnight or oil red O at 60 degrees for 12 mins as previously described¹¹. Slides were then counterstained with Harris hematoxylin (SH30, Fisher Scientific) and mounted in Aquatex (108562, Merck). Quantification of staining was performed using ImageJ software.

Flow Cytometry Analysis

iNKTs and Macrophages from Tissues:

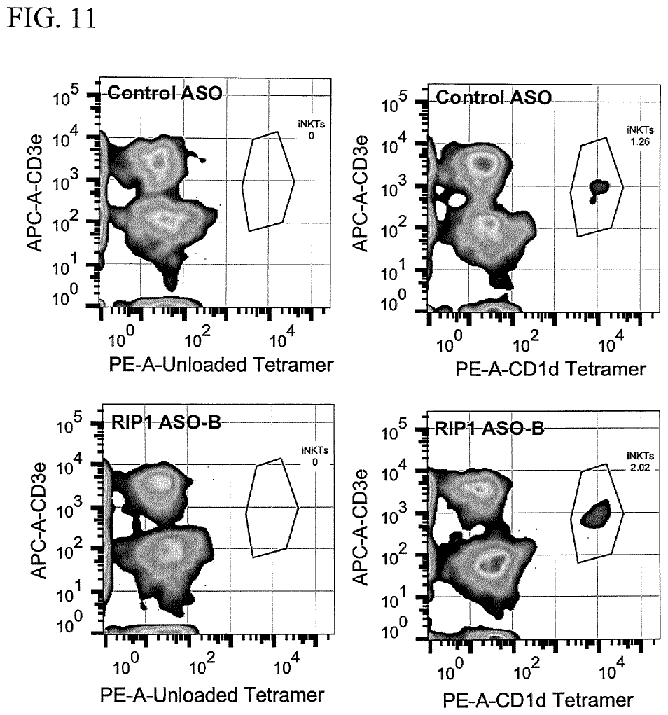

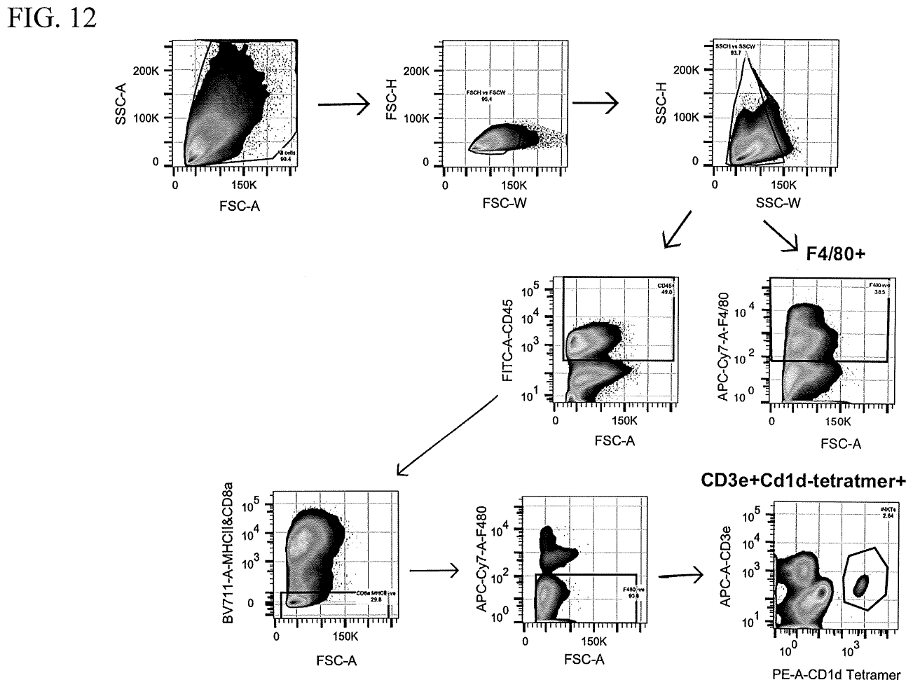

iNKTs were isolated as previously described³⁷. Briefly, cells were digested from adipose tissue using collagenase type IV or dissociated from spleen using 2 glass slides and the single-cell suspensions were passed through a 40 um mesh filter. The cell suspensions were first blocked with FBS, unlabelled streptavidin, Fc receptor blocking antibody (CD16/CD32) and BV711-labelled CD8a and MHCII (BD Biosciences), then incubated with PE-labelled-PBS57-CD1d tetramers (courtesy of the NIH Tetramer Core Facility). Unloaded CD1d tetramers were used as negative controls (FIG. 11). Cells were also labelled with APC-Cy7-labelled F4/80 (Biolegend), APC-labelled CD3e, and FITC-labelled CD45 (BD Biosciences). Mean fluorescence intensity was determined by flow cytometry. Gating strategies to detecting iNKT and F4/80 populations are shown in FIG. 12.

Circulating Blood Leukocytes:

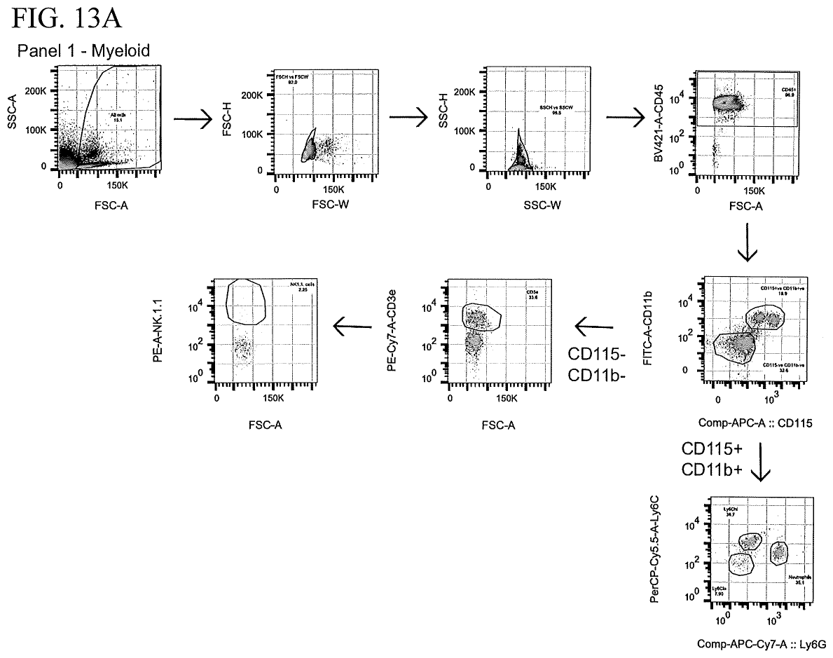

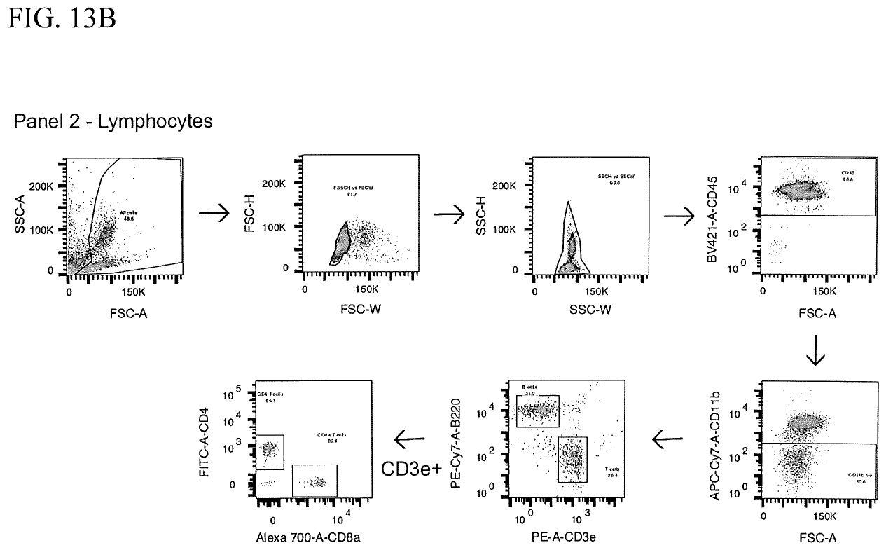

Mouse blood was collected in heparin-coated tubes and RBCs were lysed in lysis buffer (150 mM NH₄Cl, 10 mM KHCO₃ and 0.1 mM Na₂EDTA, pH7.4) for 5 mins on ice. Leukocytes were isolated via centrifugation at 340 g for 10 mins and the cells were then divided into 2 and stained with 2 antibody panels (1:100): (i) BV421-CD45, FITC-CD11b, PerCP5.5-Ly6C, APC-Cy7-Ly6G, APC-CD115, PE-NK.1.1, Pe-Cy7-CD3e, and (ii) BV421-CD45, FITC-CD4, Alexa 700-CD8a, PE-CD3e, Pe-Cy7-B220, APC-Cy7-CD11b. Cells were washed and analysed for mean fluorescence intensity using FACS Aria III (BD Biosciences). Gating strategies are shown in FIGS. 13A and 13B.

Statistical Analysis. Comparisons between 2 groups were performed using a two-tailed Student's t-test and multiple groups were compared using a one-way ANOVA for single parameters, or two-way ANOVA for multiple parameters (GraphPad Prism). Statistical significance were denoted by an * for p<0.05, ** for p<0.01, *** for p<0.001 and **** for p<0.0001.

### Example 1: Genetic Variants in the RIPK1 Locus Associate with Obesity and Higher Adipose RIPK1 Expression

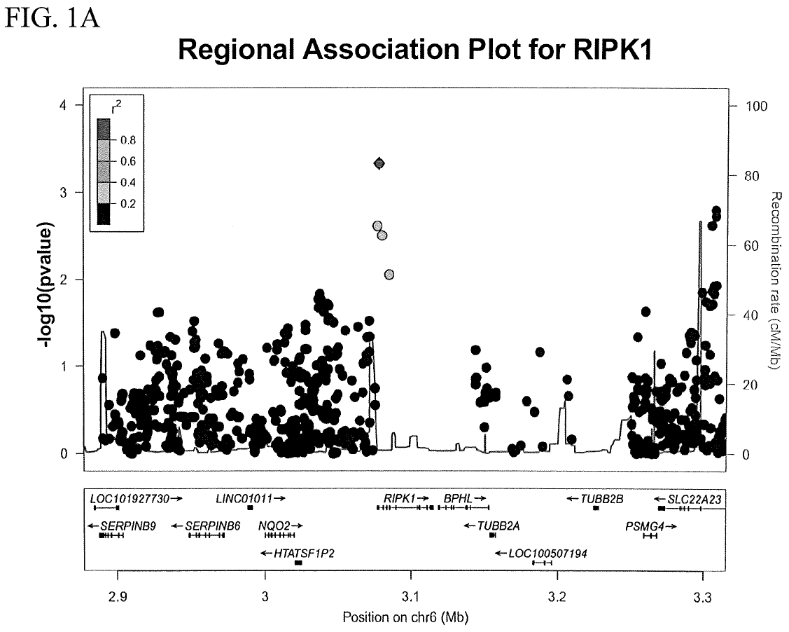

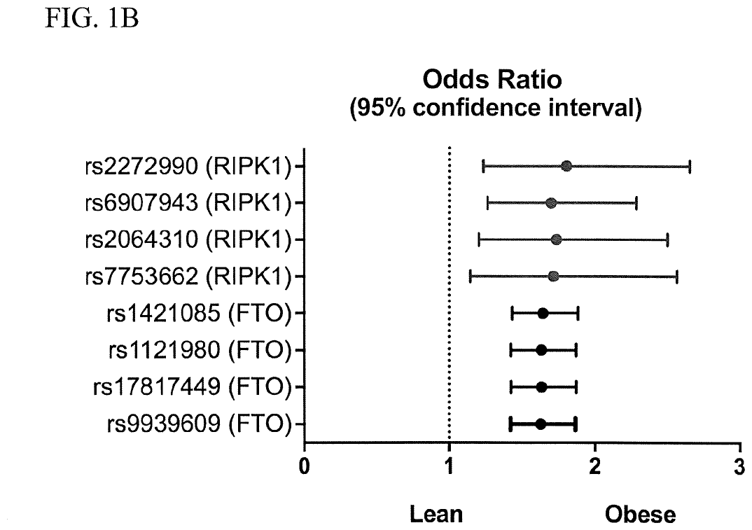

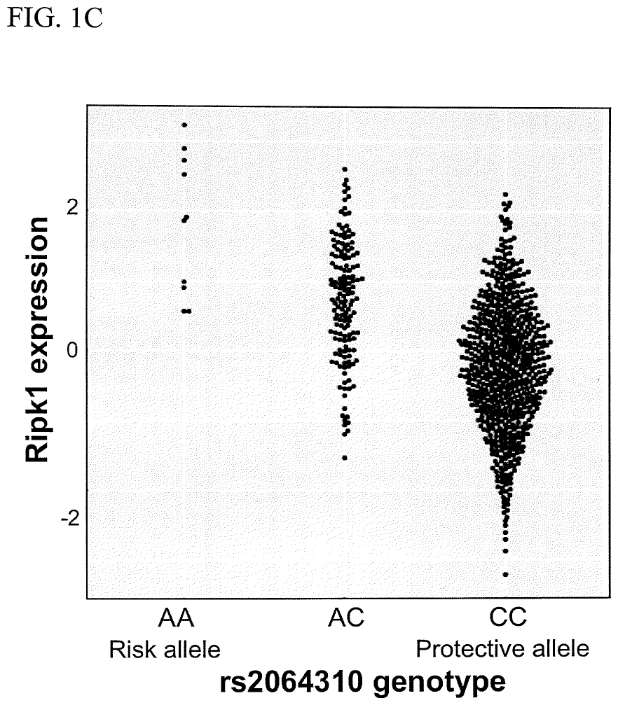

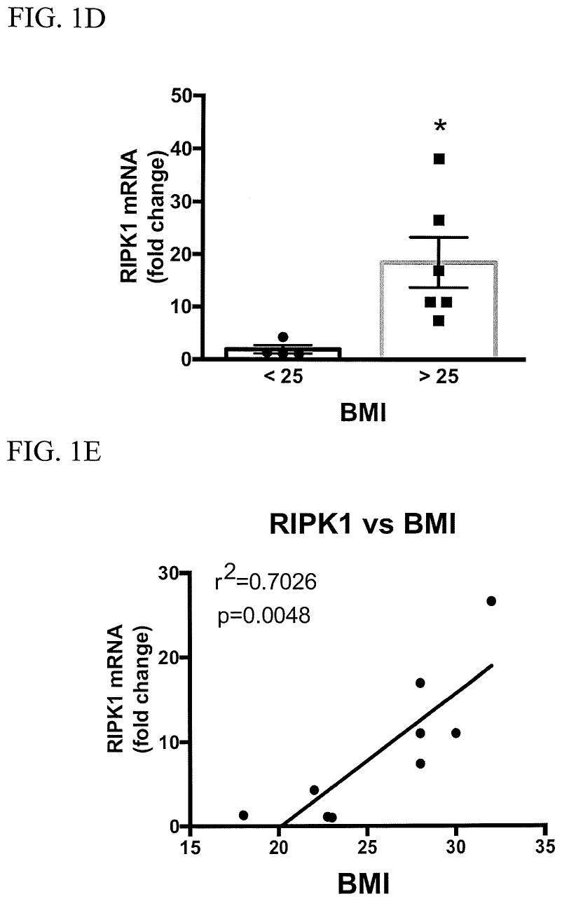

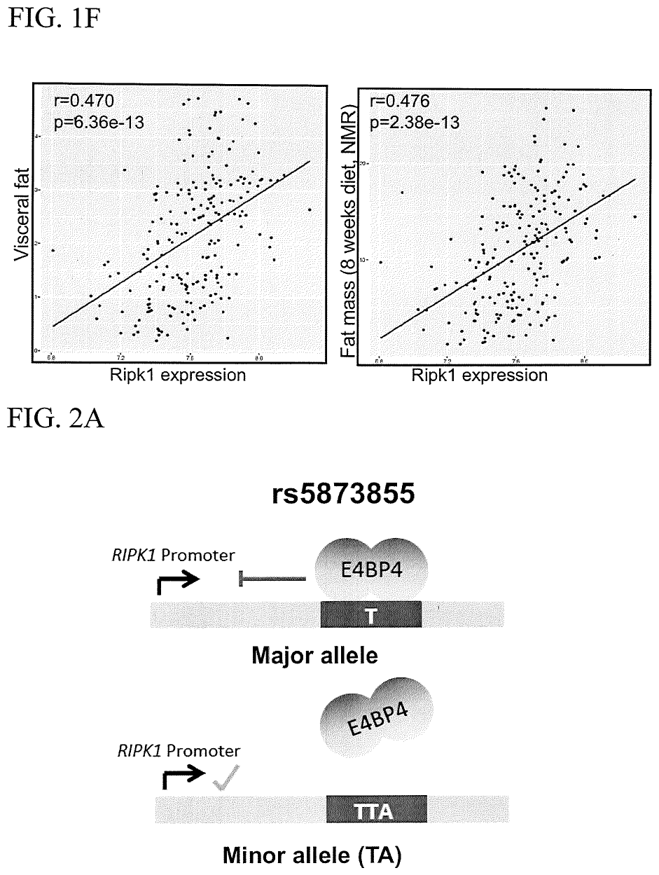

We began by asking whether the human RIPK1 gene associates with obesity. First, we interrogated the Ottawa OBese vs LEan study cohort (OBLE) consisting of unrelated extremes of lean and obese subjects of European ancestry¹⁵ (study characteristics in Table 1). After adjusting for age, sex and the first two principal components of ancestry, we identified a SNP at the RIPK1 locus that was strongly associated with obesity (r56907943, p=4.71×10⁻⁴, SEQ ID NOs: 4, 29) (FIG. 1A). rs6907943 is linked (r²>0.6) with seven other common SNPs (Haploreg v4.1, Table 2). As expected, these linked SNPs showed a similar adjusted odds ratio for risk of obesity (1.75-1.89, FIG. 1B, top). The magnitude of risk associated with RIPK1 polymorphisms and obesity in the OBLE study was comparable to that of well-documented SNP associated with obesity in the FTO locus (e.g. rs1121980, SEQ ID NO: 11, FIG. 1B, bottom)¹⁶. FTO locuses listed in FIG. 1B include: rs1421085 (SEQ ID NO: 10), rs1121980 (SEQ ID NO: 11), rs17817449 (SEQ ID NO: 12), and rs9939609 (SEQ ID NO: 13).

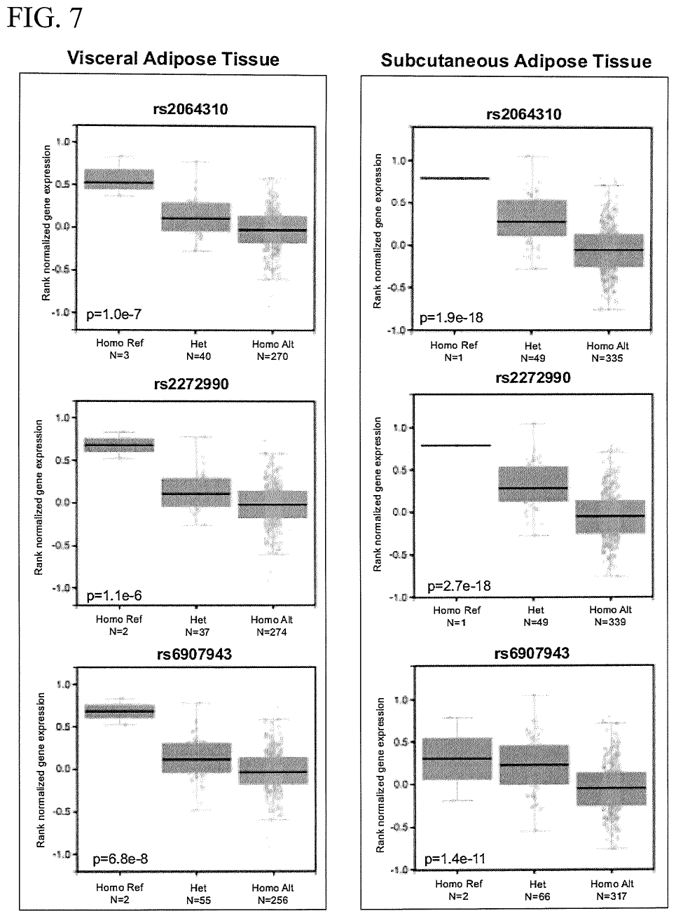

We next asked whether polymorphisms in the RIPK1 region may impact the expression of RIPK1 in adipose tissue. Interrogating the METSIM cohort of 770 individuals for expression quantitative trait loci (eQTL) in the RIPK1 region, we found that rs6907943 (SEQ ID NO: 4) is a strong eQTL for RIPK1 mRNA in subcutaneous adipose tissue (p=5×10⁻⁹, FIG. 1C)^{17,18}. A similar trend was observed for the other related SNPs, where individuals carrying the minor allele of the SNP (7-10% of the population) have increased RIPK1 expression in subcutaneous adipose tissue (p-value range=10⁻⁵ to 10⁻¹⁴). These data are concordant with those from the Genotype-Tissue Expression (GTEx) database, where the minor alleles for rs2064310 (SEQ ID NO: 5), rs2272990 (SEQ ID NO: 3) and rs67907943 (SEQ ID NO: 4) associate with higher expression of RIPK1 in adipose tissue from both visceral and subcutaneous depots (FIG. 7)¹⁹. In FIG. 7, p-values are as calculated by the GTEx Consortium detailed at: https://www.gtextportal.org/home/documentationPage#AboutGTEx. In a cohort of subjects categorized as overweight (BMI >25) RIPK1 mRNA in adipose tissue was significantly elevated compared to normal weight subjects (BMI <25; p<0.001, FIG. 1D) and correlated positively with BMI (R²=0.70, p<0.01, FIG. 1E).

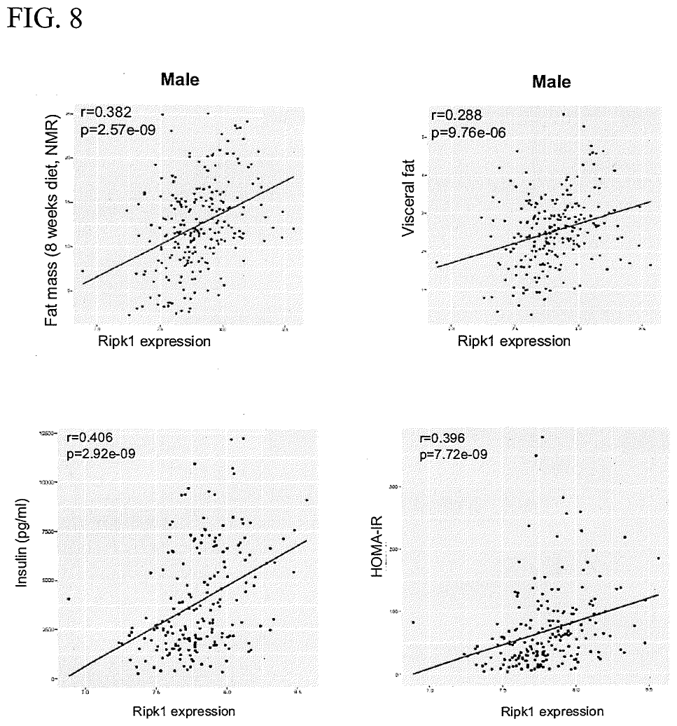

We further examined the expression of Ripk1 mRNA in the Hybrid Mouse Diversity Panel, which contains transcriptome data from approximately 100 inbred strains of mice²⁰. The expression of Ripk1 in adipose tissue from high-fat fed mice correlated strongly with total fat mass assessed by NMR and visceral fat mass at sacrifice in both male and female mice (FIGS. 1F, 8A). Moreover, adipose Ripk1 expression significantly correlated with other metabolic traits including circulating insulin levels and HOMA-IR, a marker of insulin resistance, in high-fat fed male mice (FIG. 8B). Together these data demonstrate that obesity-associated polymorphisms in the RIPK1 gene functionally result in elevated RIPK1 expression in adipose tissue in humans, and elevated Ripk1 expression in mice strongly associates with adiposity and metabolic dysfunction.

In addition to adipose tissue, RIPK1 SNPs are very strong eQTLs for RIPK1 expression in other tissues including the brain, thyroid, and whole blood suggesting that alterations in RIPK1 gene expression may drive inflammation in other tissues. Although other genome-wide association studies investigating obesity have not reported RIPK1 association with obesity at the genome-wide level (p≤5×10−8), specifically interrogating our OBLE dataset of extremes of body mass index revealed a strong but non-genome wide significant association with obesity that is similar in magnitude to the well-replicated FTO SNP.

TABLE 1
Obese-Lean (OBLE) Study characteristics of the Ottawa OBese
vs LEan study cohort (OBLE) consisting of unrelated extremes of
lean and obese subjects of European ancestry¹⁴.

| | n | % females | Mean age (SE) | Mean BMI (SE) |
|---|---|---|---|---|
| Lean | 869 | 60.6 | 44.4 (0.5) | 20.31 (0.1) |
| Obese | 958 | 71.1 | 46.33 (0.3) | 43.11 (0.3) |

TABLE 2
List of variants at the RIPK1 locus in strong to moderate
linkage disequilibrium (r² > 0.6) with the obesity-
associated rs6907943 SNP (SEQ ID NO: 4). Linkage
data is taken from the HaploReg website
(version 4.1).

| | | | Common/ | | | |
|---|---|---|---|---|---|---|
| Variant/SEQ | Position | | Protective | Rare/Risk | | |
| ID NO. | (hs19) | MAF | Allele | Allele | D′ | R² |
| rs67432438/ | chr6: 3074964 | 0.10 | C | CAGTC | 0.94 | 0.83 |
| SEQ ID NOs: | | | | | | |
| 1, 26 | | | | | | |
| rs4959774/ | chr6: 3076336 | 0.14 | A | G | 0.97 | 0.63 |
| SEQ ID NOs: | | | | | | |
| 2, 27 | | | | | | |
| rs2272990/ | chr6: 3077141 | 0.08 | C | T | 0.98 | 0.72 |
| SEQ ID NOs: | | | | | | |
| 3, 28 | | | | | | |
| rs6907943/ | chr6: 3078266 | 0.10 | A | C | 1 | 1 |
| SEQ ID NOs: | | | | | | |
| 4, 29 | | | | | | |
| rs2064310/ | chr6: 3080324 | 0.08 | A | C | 1 | 0.75 |
| SEQ ID NOs: | | | | | | |
| 5, 30 | | | | | | |
| rs7753662/ | chr6: 3085129 | 0.07 | T | G | 1 | 0.71 |
| SEQ ID NOs: | | | | | | |
| 6, 31 | | | | | | |
| rs5873855/ | chr6: 3086051 | 0.07 | T | TTA | 0.96 | 0.68 |
| SEQ ID NOs: | | | | | | |
| 7, 32 | | | | | | |
| rs141325626/ | chr6: 3086310 | 0.07 | C | 12-mer | 0.98 | 0.69 |
| SEQ ID NOs: | | | | | | |
| 8, 33 | | | | | | |

We identify herein the first evidence of a genetic link between inflammation and obesity in humans. We find a genetic variant in humans that strongly associates with elevated RIPK1 in adipose tissue also confers significant risk for obesity. In both mice and humans, the expression level of RIPK1 is elevated in adipose tissue in the setting of obesity, and silencing RIPK1 dramatically reduces adiposity and promotes improved metabolic function. Our work suggests that RIPK1 directly induces activation of pro-inflammatory signaling in adipose tissue, which promotes accumulation of macrophages and drives metainflammation. We show that genetic polymorphisms near the human RIPK1 locus associate with increased RIPK1 gene expression in adipose tissue and are strongly linked with the risk of obesity in a human population.

### Example 2: The RIPK1 Polymorphism Rs5873855 (SEQ ID NO: 7) Disrupts a Transcriptional Repressor Binding Site

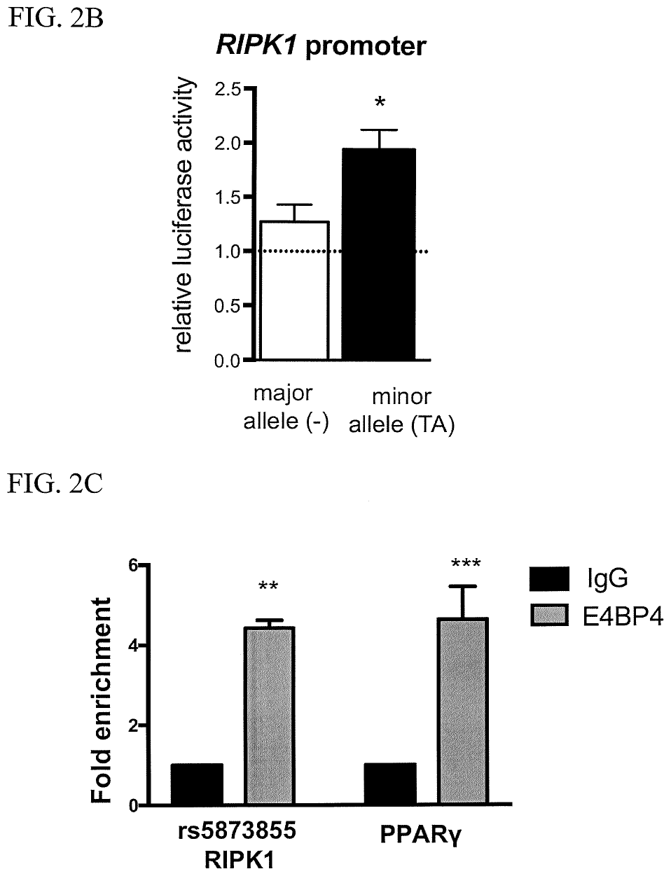

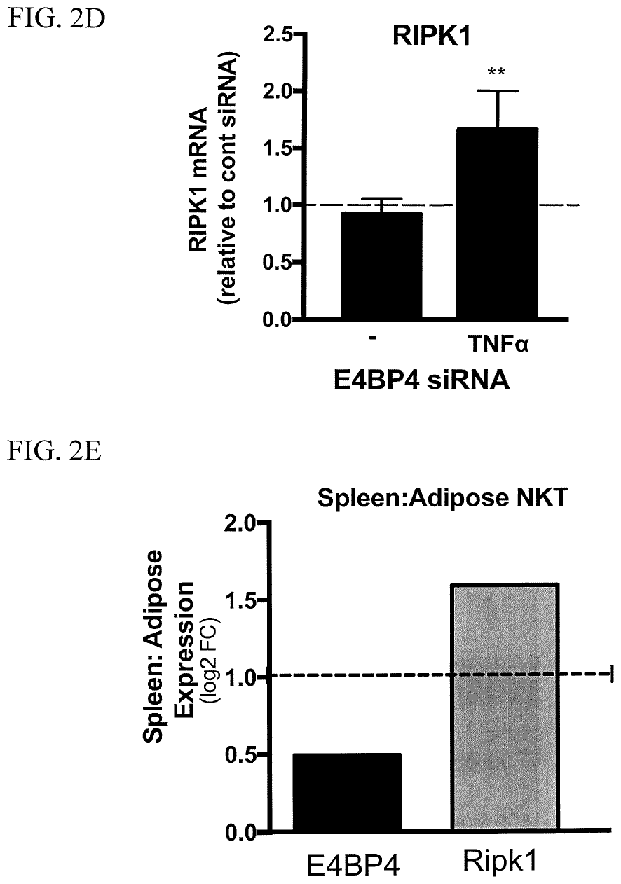

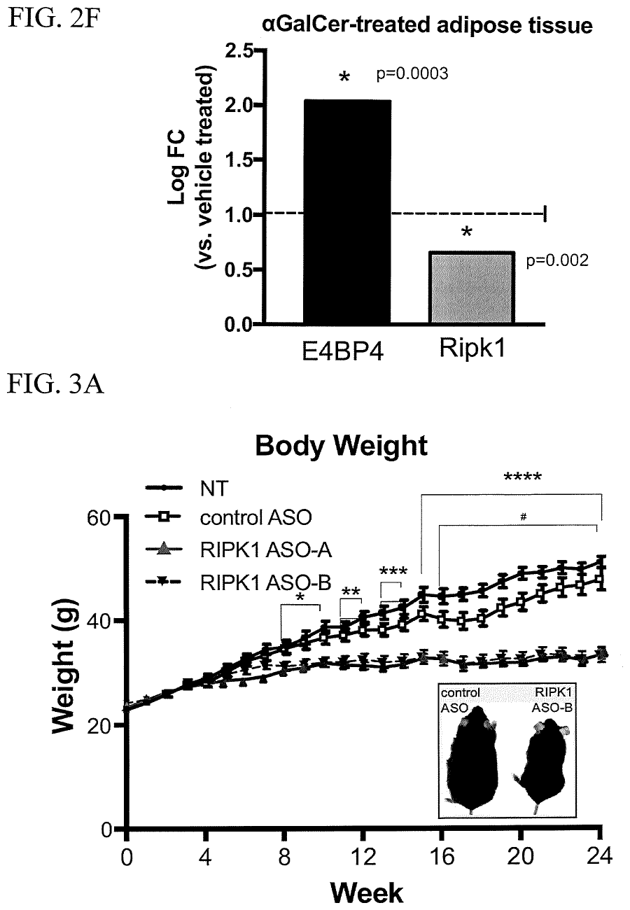

We postulated that one or more of these SNPs may disrupt a gene regulatory element(s) that subsequently alters the transcription of RIPK1. One lead candidate SNP rs5873855 (Table 2) is in the binding site for a well-characterized transcription factor, E4 promoter-binding protein 4 (E4BP4, also known as NFIL3)²¹. E4BP4 is a repressive transcription factor that has been shown to control invariant natural killer T cell function²² and is induced during M1 and M2 macrophage polarization¹⁰. The deletion at rs5873855 is predicted to allow E4BP4 binding and repression, whereas the obesity-associated TA insertion is rare and is predicted to disrupt the E4BP4 binding site to de-repress RIPK1 transcription and expression (FIG. 2A). To test the effects of the obesity-associated TA insertion in rs5873855 we generated a reporter construct containing the human RIPK1 promoter upstream of the firefly luciferase gene and a 309 bp genomic DNA sequence containing both the rs5873855 (−) and (TA) alleles downstream. RIPK1 promoter activity was assessed in a human adipocyte liposarcoma cell line, SW872 cells. When compared to the major (common) allele, the presence of the obesity risk rs5873855 (TA) allele had greater luciferase activation (FIG. 2B), in keeping with notion that this variant may disrupt E4BP4 binding and lead to de-repression of RIPK1 promoter activation. We next assessed whether endogenous E4BP4 is capable of binding the intronic sequence containing the obesity-associated SNPs. Chromatin immunoprecipitation assays confirmed that E4BP4 binding was enriched at the rs5873855 sequence in the RIPK1 reference allele, similar to what has been shown with binding to other E4BP4 target genes, such as PPARγ (FIG. 2C)²³. Indeed, silencing of E4BP4 with siRNA leads to the activation of RIPK1 mRNA expression, at both basal and TNFα-stimulated conditions (FIG. 2D). Together, these data are the first to show a role for E4BP4 in the regulation of RIPK1 expression.

E4BP4 has an important role in regulating invariant natural killer T (iNKT) cell function in adipose tissue. iNKT cells in the adipose tissue protect against diet-induced obesity²⁴, and E4BP4 is required for the production of IL-10²² to drive M2 macrophage polarization²⁵. Given that E4BP4 serves as a transcriptional repressor of RIPK1, we postulated that the levels of RIPK1 and E4BP4 may be inversely correlated in adipose tissue iNKT cells. Previous studies in mice profiling the gene expression in iNKT cells from adipose tissue and other depots (spleen, liver) enabled us to examine the relationship between Ripk1 and E4bp4 expression using publicly available transcriptome data. E4bp4 expression is higher in adipose tissue iNKTs compared with iNKTs from spleen, whereas Ripk1 expression was low in adipose and high in splenic iNKTs (FIG. 2F, from dataset GSE63358). We next analyzed available data from adipose tissue of obese mice treated with or without αGalCer, a lipid antigen specific for activation of iNKTs²⁵. This activation increases E4bp4 expression, and concomitantly reduces Ripk1 expression (FIG. 2G, from dataset GSE36032, WT vehicle vs. WTαGalCer). Together, these data further confirm RIPK1 as a downstream target of E4BP4 in adipose tissue iNKT cells.

The regulatory mechanisms governing RIPK1 transcriptional activation are relatively unknown. We identified the promoter of human RIPK1 located approximately 5 kb upstream of the previously annotated transcriptional start site, and a putative regulatory element in the RIPK1 gene in intron 5 that harbours SNPs associated with increased risk of obesity. Indeed, the minor allele for rs5873855 (SEQ ID NO: 7), with a frequency of 0.08, contains the insertion TTA in a region predicted to be bound by the transcription factor E4BP4. We find E4BP4 bound to this site, and the presence of the TTA insertion enhances the activation of the RIPK1 promoter by E4BP4, suggesting E4BP4 binds to and represses this region. Although rs5873855 represented a good RIPK1 functional candidate, we cannot rule out effects of the other linked SNPs as regulators of RIPK1 expression. We show that one of these SNPs is within a binding site for E4BP4 and increases RIPK1 promoter activity and RIPK1 gene expression in adipose tissue.

### Example 3: Inhibition of Ripk-1 Reduces Diet-Induced Obesity and Improves Insulin Resistance

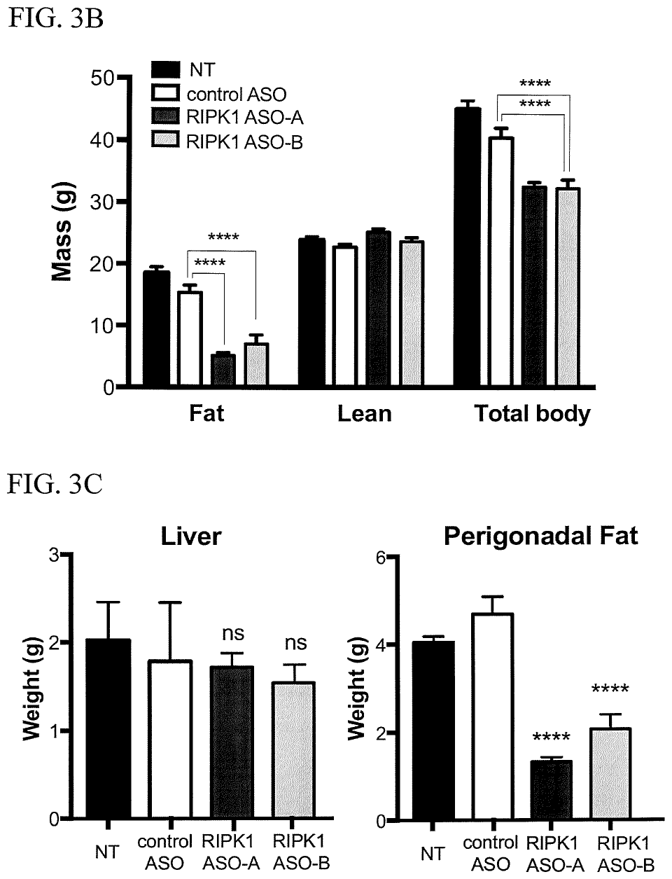

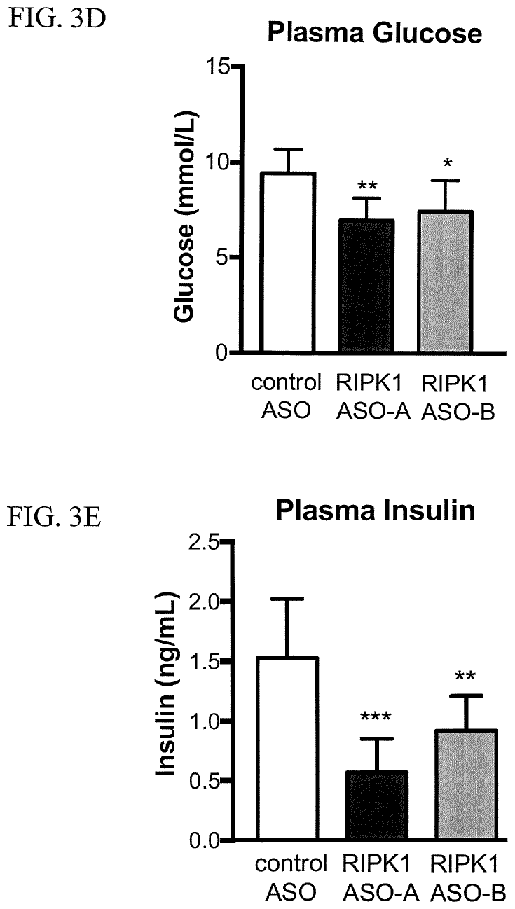

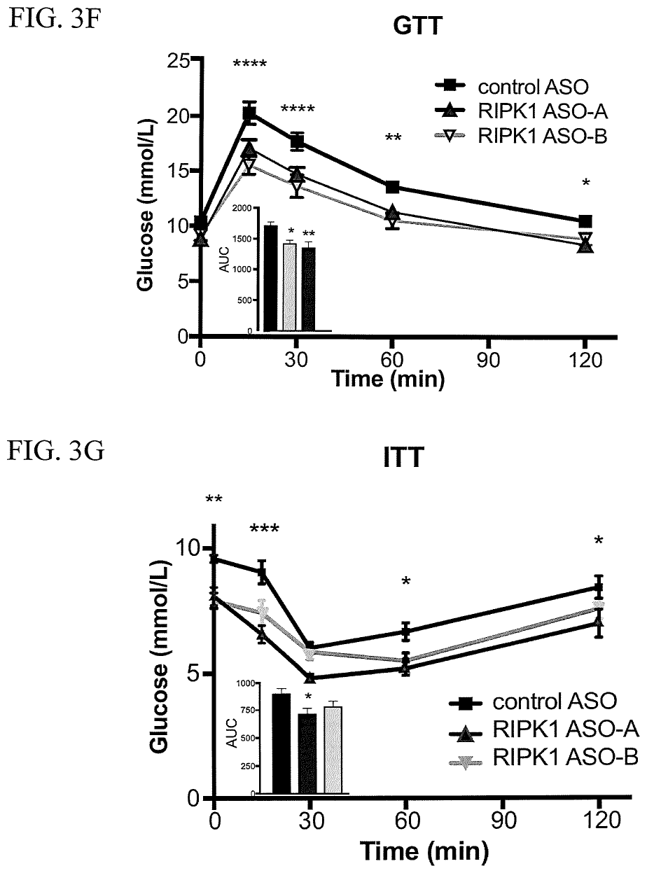

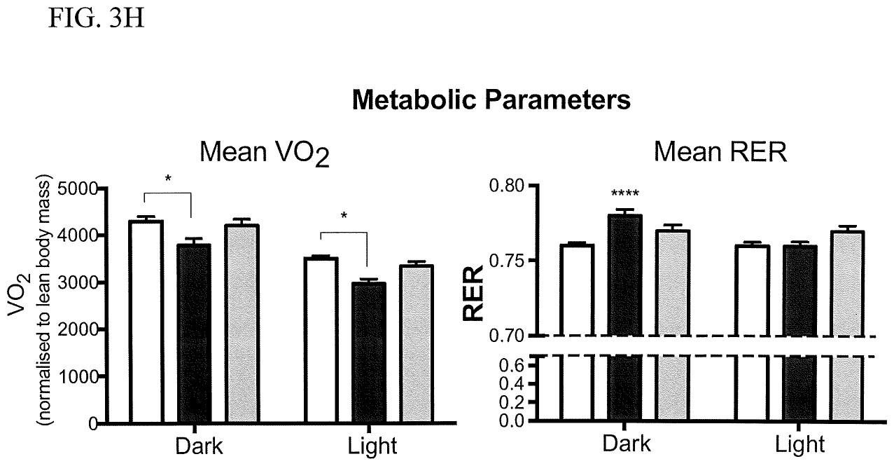

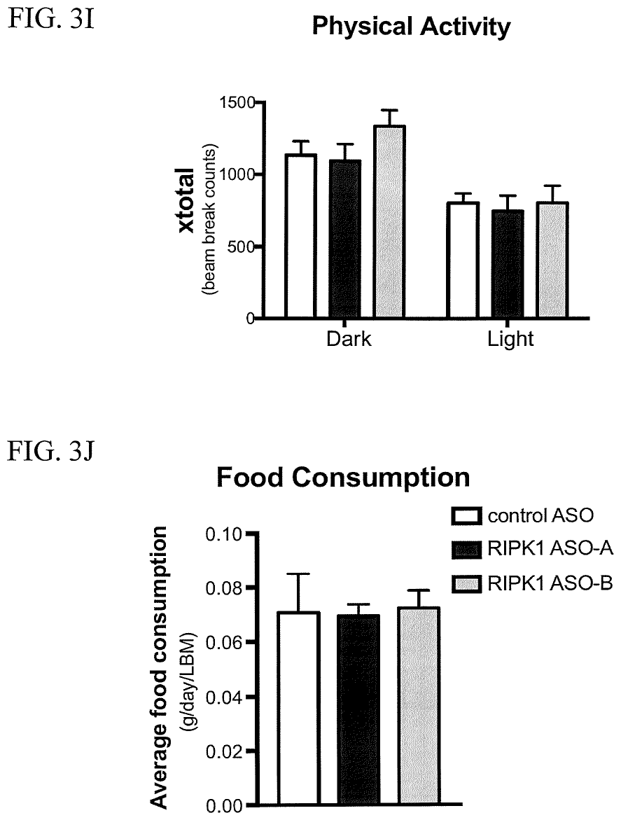

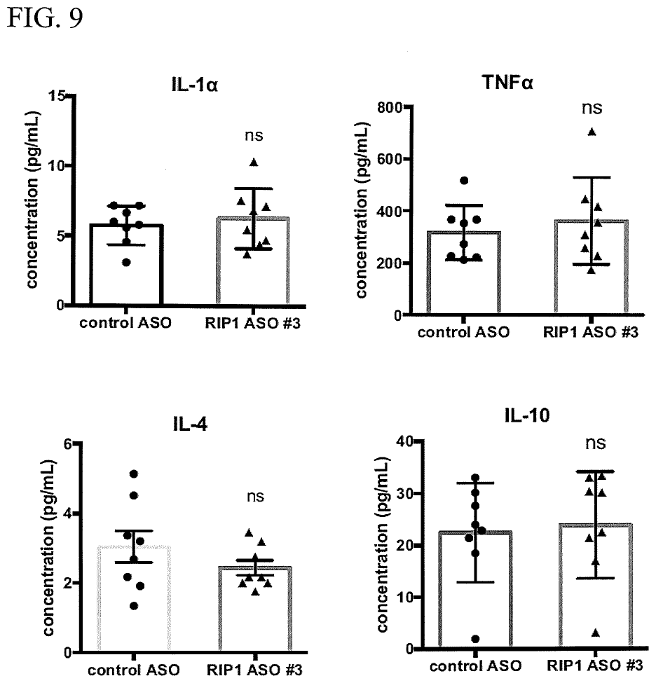

To determine whether RIPK1 expression drives obesity, we tested whether knock down of RIPK1 using anti-sense oligonucleotides (ASOs) could rescue the adverse metabolic consequences associated with diet-induced obesity. FIG. 3 shows therapeutic knock down data of RIPK1 in a diet-induced obesity (DIO) mouse model. Male C57BL/6J mice were fed a high fat diet (60% kCal) for 24 weeks and subjected to weekly injections of control ASOs or one of two ASOs targeting RIPK1 (RIPK1 ASO-A or ASO-B). RIPK1 ASO therapy reduced whole body weight over the course of the high-fat feeding (FIG. 3A). Interestingly, this difference in body weight was a result of a substantial reduction in total fat mass assessed by NMR (15.3±3 g in control vs 5.1±1 g or 7.0±4 g in RIPK1 ASO groups, p<0.001; FIG. 3B), which was particularly evident in the perigonadal fat pads of these mice (FIG. 3C). RIPK1 ASO treatment also improved glucose homeostasis, including fasted blood glucose (FIG. 3D), plasma insulin (FIG. 3E) and response to both glucose and insulin challenge (GTT; FIG. 3F; ITT, FIG. 3G). To further examine whole body metabolic function, we performed open-circuit indirect calorimetry and found little difference in the overall metabolic rates of these mice. Mice treated with RIPK1 ASO-A had a slight decrease in VO₂ and a corresponding small increase in RER compared to both controls and RIPK1 ASO-B (FIG. 3H). Despite the reduction in body fat, there was no significant difference in the physical activity (FIG. 3I) or food consumption (FIG. 3J) in mice treated with either RIPK1 ASOs compared to controls. Data was obtained from one-way ANOVA (FIG. 3B-I) and total body weights (FIG. 3A). GTT (FIG. 3E) and ITT (FIG. 3F) were analyzed using two-way ANOVA. We next ascertained the role of RIP-1 kinase (RIPK-1) activity on the obesogenic function of RIPK1. We evaluated the response to high fat diet feeding on mice where the catalytic kinase residue of RIPK1 (Lysine 45) is mutated (K45A)²⁶, compared to WT littermates. After 20 weeks on diet, RIPK1^{K45A} mice gained equivalent weight to their WT counterparts and had similar fat mass in the epididymal WAT (FIGS. 9A, 9B). When challenged with a bolus of either glucose or insulin, mice with defective RIPK1 K45 activity responded in a similar manner to their WT littermates (FIGS. 9C, 9D). Together, these data demonstrate that silencing RIPK1 reduces body weight and fat mass and is accompanied by improved glucose tolerance and insulin sensitivity, but that this is independent of the kinase activity of RIPK1.

Variants in RIPK1 that elevate RIPK1 expression in the adipose tissue while simultaneously putting an individual at risk of being obese strongly suggests that RIPK1 is a causal factor of these effects. The data in high fat diet fed mice support this notion, where blocking RIPK1 results in a significant protection from weight gain and glucose intolerance. Together these data are the first to demonstrate hyperactivation of inflammatory signaling being a heritable and therapeutically targetable contributor to obesity and its associated complications.

### Example 4: Blocking RIPK1 Reduces Adipose Tissue Inflammation

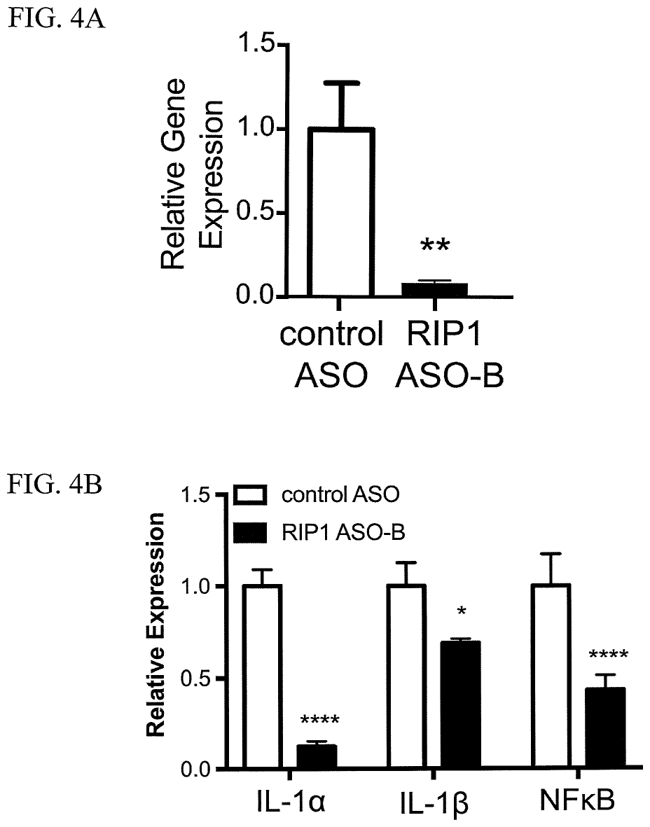

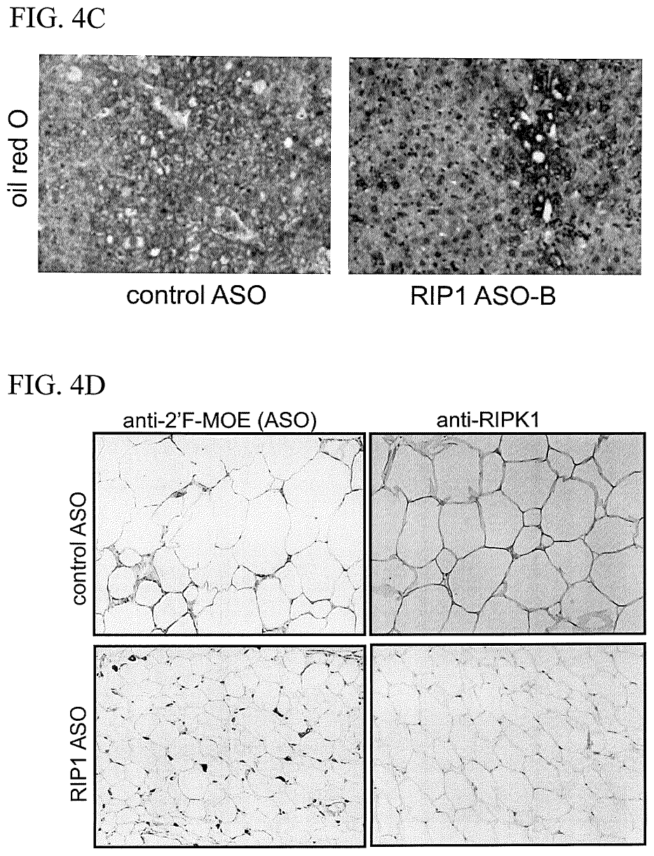

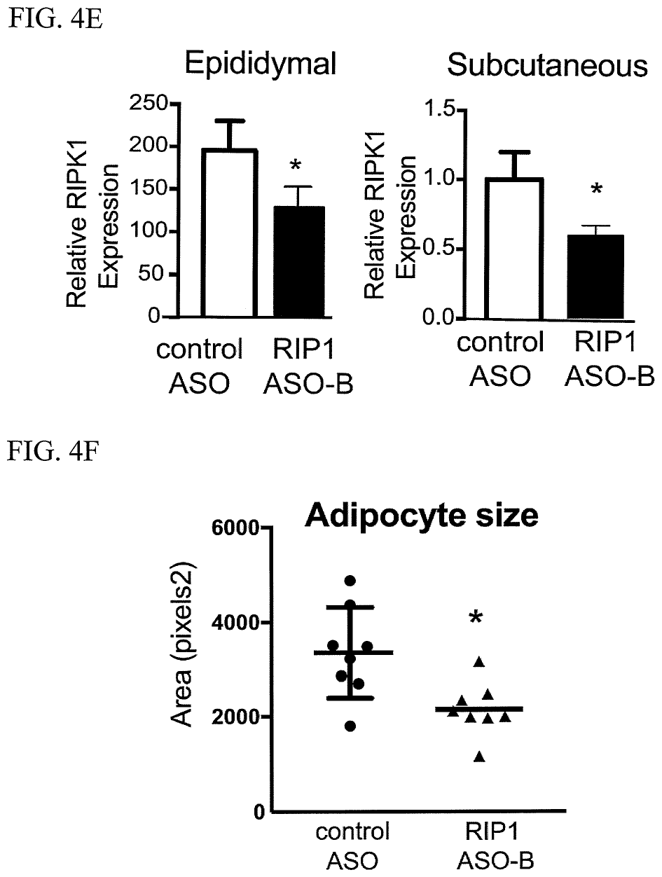

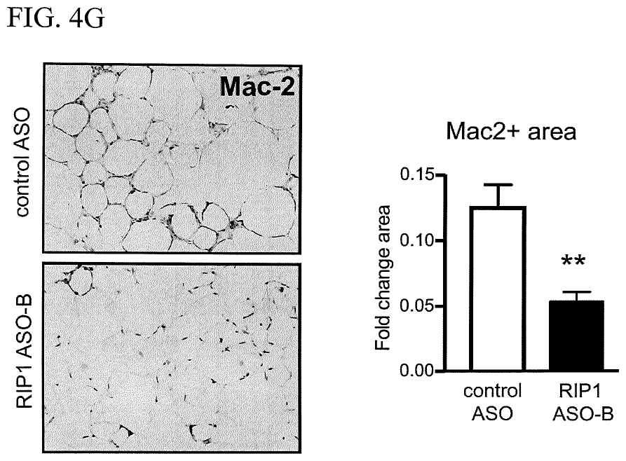

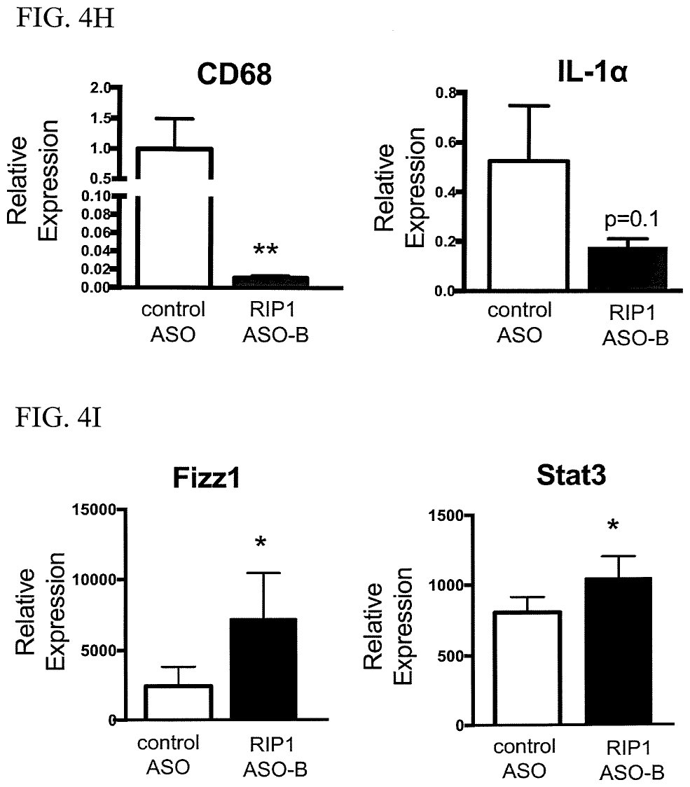

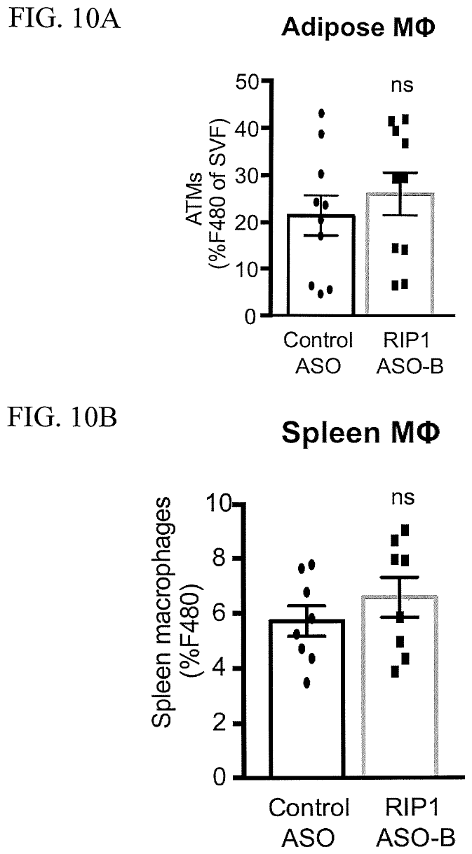

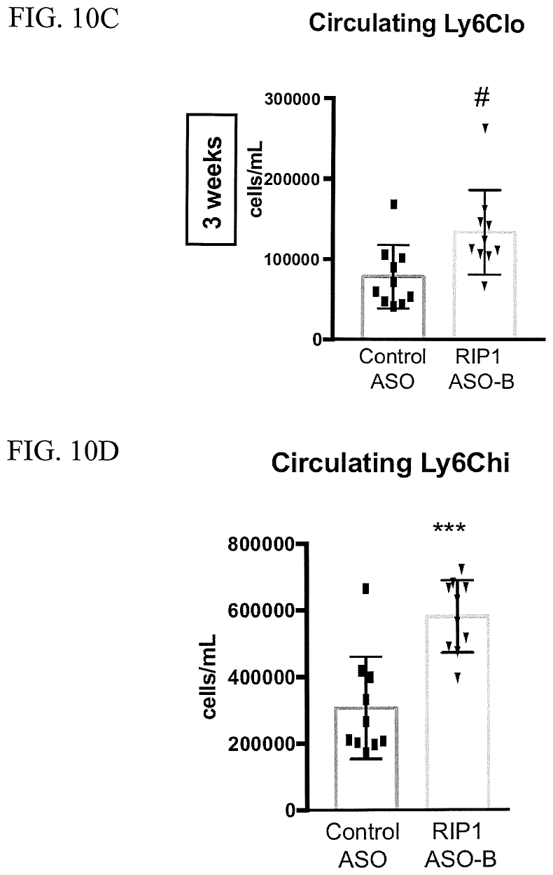

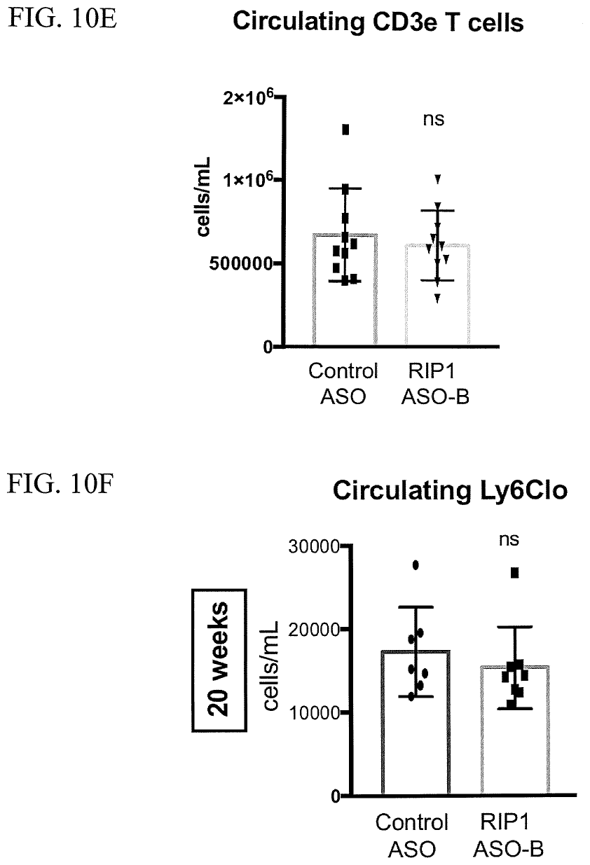

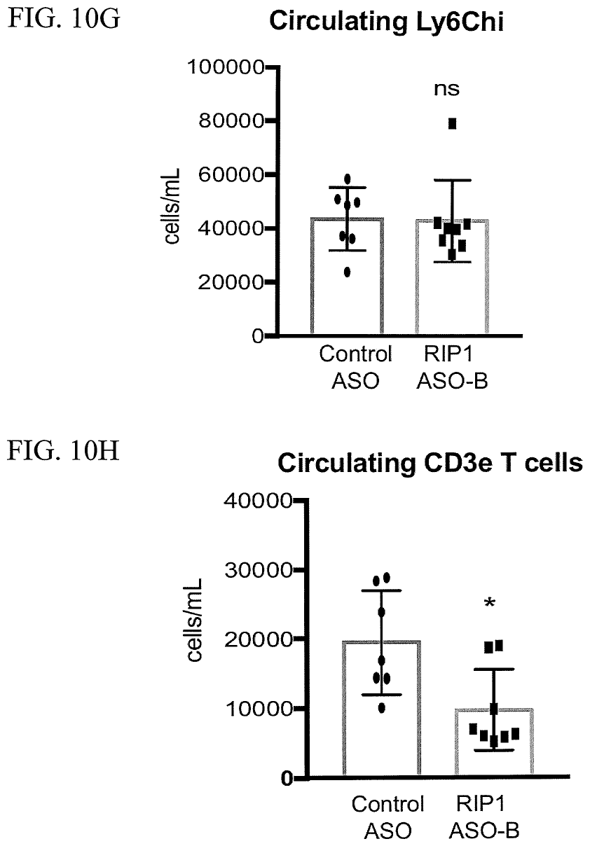

RIPK1 is an activator of multiple inflammatory pathways, including NFκB. To better understand the inflammatory status of mice treated with RIPK1 inhibitors, we examined inflammatory gene activation in liver and adipose tissue of RIPK1 ASO treated mice. FIGS. 4A-G show knockdown results of RIPK1 decreases inflammation in liver and adipose tissue. As anticipated, we saw a substantial knockdown of RIPK1 gene expression in the liver (FIG. 4A) as well as a reduction of key inflammatory genes known to be downstream of RIPK1 (FIG. 4B). Importantly, there was a dramatic decrease in lipid staining as detected by oil-red-O staining in the RIPK1 ASO-treated mice (FIG. 4C), demonstrating that RIPK1 ASO therapy reduces overall liver inflammation and lipid-accumulation—both of which are known to contribute to obesity and associated complications. In adipose tissue, immunostaining confirmed the delivery of RIPK1 ASOs to the adipose tissue (FIG. 4D) where, accordingly, RIPK1 protein and mRNA expression were markedly reduced (FIGS. 4D, 4E). Strikingly, we observed a decrease in both adipocyte size (FIG. 4F) and macrophage accumulation (FIG. 4G) in RIPK1 ASO-treated mice. RIPK1 governs activation of NFκB and elicits a pro-inflammatory response, therefore we tested downstream activation of inflammatory genes in RIPK1 ASO-treated mice. Expression analysis of epididymal adipose tissue revealed a downregulation of pro-inflammatory genes in the adipose tissue of RIPK1 ASO vs. control ASO treated mice, including CD68 and IL-1α (FIG. 4H) whereas genes associated with M2 macrophage polarization Fizz1 and Stat3 were elevated when RIPK1 was knocked down (FIG. 4I). Of note, there were no detectable differences in circulating cytokines in RIPK1 ASO treated compared to control treated mice, indicating that the changes in inflammatory gene activation were localized to the adipose tissue and did not represent a global defect in inflammatory signaling (FIGS. 10A-H). These data indicate that RIPK1 inhibition reduces activation of inflammatory pathways known to be downstream of RIPK1 in the liver and adipose tissue of obese mice.

RIPK1 is a member of a family of kinases that includes RIPK3, which have divergent functions in inflammation. RIPK1 is modified by its reciprocal ubiquitination and phosphorylation status²⁸. Ubiquitinated RIPK1 promotes NF|B activation whereas phosphorylated RIPK1 acts to activate RIPK3 to promote programmed necrosis^{13,29,30}. Complete genetic ablation of RIPK1 results in embryonic lethality, as RIPK1 appears to perform a scaffolding function during development³¹. When the kinase domain of RIPK1 is mutated, mice have reduced TNF-dependent necroptosis in vitro and in vivo, owing to the dependence of RIPK1 kinase activity in the execution of programmed necrosis²⁶. We find that the kinase activity of RIPK1 (K45) is dispensable for RIPK1 in promoting obesity. Our data suggests that the activation of NFκ-B by RIPK1 mediates the pro-inflammatory and pro-obesogenic function in the adipose tissue, however further mechanistic studies are needed to define this. This is further supported when contrasting the current study with genetic loss of RIPK3, which has been shown to exacerbate diet-induced obesity and insulin resistance³². This is attributed to the expansion of adipocytes in the absence of RIPK3-dependent adipocyte apoptosis and turnover. With antisense knockdown of RIPK1 in the present study, we see a decrease in adipocyte size and decreased macrophage accumulation in adipose tissue of RIPK1 ASO treated mice, arguing against excessive cell death in this model. Rather, silencing RIPK1 reduces pro-inflammatory NFκB gene expression specifically within the adipose tissue environment. These data suggest that the roles for RIPK1 and RIPK3 in controlling adipocyte cell death are distinct. Notably, loss of RIPK1 in intestinal epithelial cells and keratinoctyes can promote both necroptosis and apoptosis, exacerbating inflammation and a loss of barrier function³³. This was attributed to the dominant role for RIPK1 as a scaffold for pro-survival proteins to inhibit cell death in barrier tissues. In our mice treated with RIPK1 ASOs, we did not observe evidence of skin lesions or intestinal abnormalities, and nutrient absorption was similar in both control and RIPK1 groups (data not shown). These data suggest that while ASOs do indeed target epithelial layers like the small intestine, they may allow sufficient expression of RIPK1 for barrier tissue function to remain intact in these mice.

### Example 5: RIPK1 Inhibition Augments Invariant Natural Killer T-Cells in Obese Mice

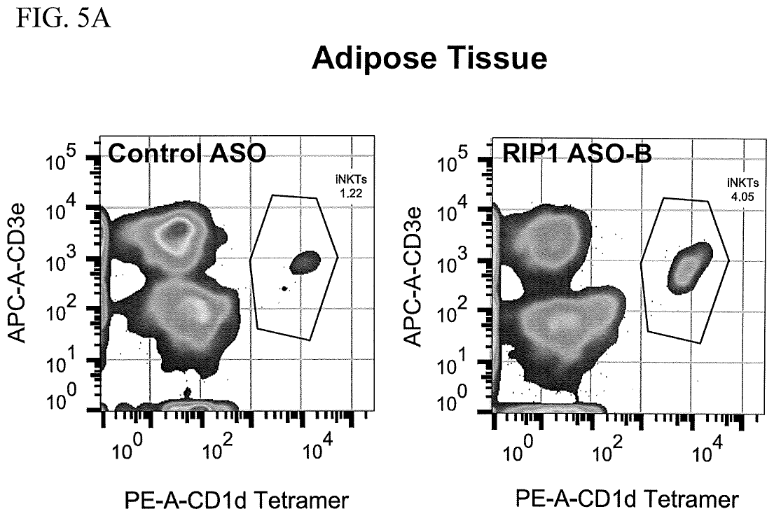

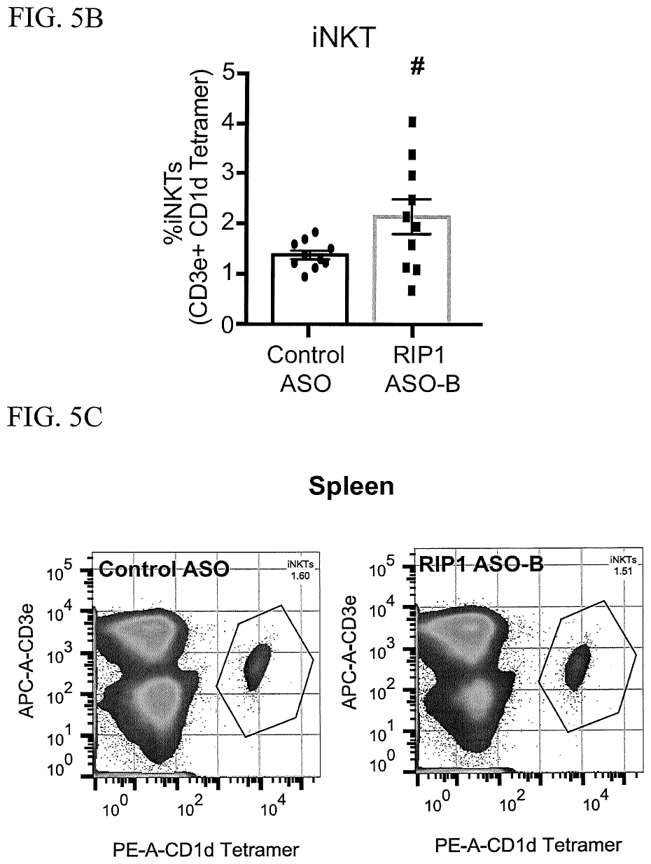

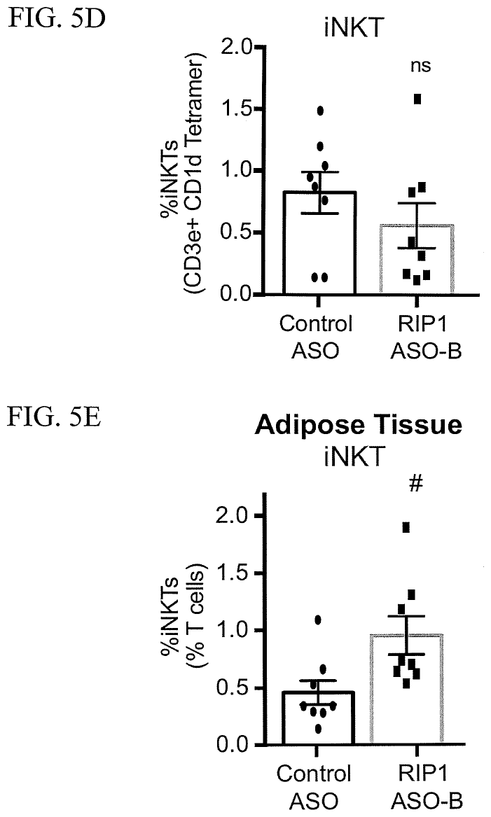

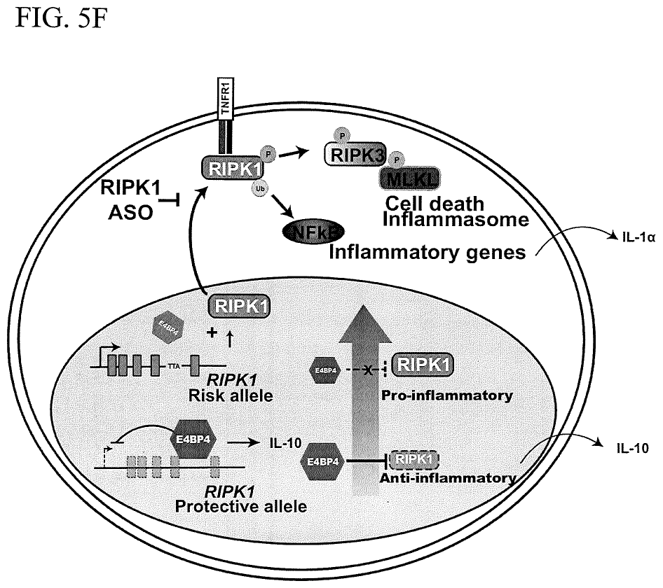

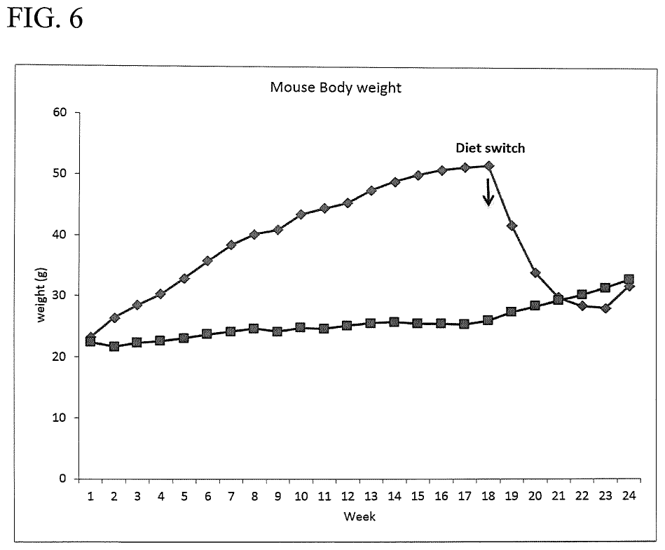

Given that E4BP4 represses RIPK1 expression, and we observed a strong inverse correlation between RIPK1 levels and E4BP4 levels in iNKT cells, we hypothesized that RIPK1 inhibition may impact iNKTs in adipose tissue. FIGS. 5A-D show flow cytometry analysis of the isolated stromal vascular fraction (SVF) of adipose tissue from mice treated with cont ASO or RIP1 ASO-B for 3 weeks while fed a high fat diet. Identification of iNKTs using a Cd1d-tetramer, we observed an increase in adipose tissue iNKT cells after short-term high fat feeding upon RIPK1 inhibition compared to controls (CD3e⁺Cd1d-tetramer⁺, FIG. 5A, B). These differences were unique to the adipose tissue, as the proportion of spleen iNKTs was not different between groups (FIG. 5C). The increased proportion of iNKTs in RIPK1 ASO treated mice persisted after long-term high fat feeding (FIG. 5D). There were no changes in circulating T-cells in mice treated with RIPK1 ASOs compared to control ASOs, and in fact circulating CD3+ lymphoid cells were decreased after 22 weeks high-fat feeding (FIG. 11) suggesting that these effects on iNKT cells were not a result of a global increase in T-cell populations. These data suggest that RIPK1 inhibition can protect from diet-induced obesity by maintaining the anti-obesogenic population of iNKT cells uniquely within the adipose tissue.

We found a role for invariant natural killer T-cells in modulating the effects of RIPK1 in obesity. Upon activation, iNKT cells play a unique immunomodulatory function by production of either Th1-like or Th2-like cytokines³⁴. In adipose tissue, resident iNKTs recognize lipid antigens presented by the MHCI-like CD1d on adipocytes to promote IL-10, IL-13 and IL-4 production to maintain an anti-inflammatory state^{35,36} and the majority of studies show iNKTs protect against obesity^{24,37,38}. In humans, iNKT cells are enriched in adipose, but decline sharply during obesity^{24,39}. The pro- or anti-inflammatory role of iNKTs is highly tissue dependent, and a comparison of gene expression signatures between splenic, liver and adipose iNKTs suggests these compartments may have distinct populations^{22,40}. Indeed, we observed high expression of RIPK1 in splenic iNKTs in comparison to levels found in adipose iNKTs, in alignment with the pro-inflammatory nature of the spleen iNKT reservoir. In contrast, in adipose iNKT cells activated with the lipid antigen αGalCer expression of E4BP4 increases while RIPK1 expression decreases, also in agreement with their anti-inflammatory state. We observe a significant increase in iNKT number in the adipose tissue of mice treated with RIPK1 inhibitors compared to controls, which was evident as early as 3 weeks after high fat feeding, and persisted to the end of the study. This suggests that elevated RIPK1 can drive adipose tissue inflammation via changes in iNKT function, and could explain the anti-inflammatory milieu of the RIPK1 ASO-treated adipose tissue.

Our study identifies RIPK1 as an important genetic driver of immunometabolism in mice and humans. In humans, we reveal a novel genetic risk factor associated with elevated RIPK1 gene expression that puts individuals at risk for the development of obesity. In adipose tissue, RIPK1 expression correlates with adiposity and measures of glucose intolerance and, as such, RIPK1 inhibition dramatically reduces body weight, adipose tissue accumulation and improves insulin sensitivity, suggesting that elevated RIPK1 might be a central inflammatory driver of obesity. Importantly, these findings may assist in the translation of RIPK1 inhibitors, which are currently in phase 2a clinical studies for psoriasis, rheumatoid arthritis, and ulcerative colitis⁴¹. Because therapeutically blocking RIPK1 prevents the activation of local macrophage inflammatory pathways, and this improves the local inflammatory milieu to dampen metabolic dysfunction associated with high fat diet, this may be valuable for the treatment of the expanding epidemic of obesity and type 2 diabetes. Therapeutic silencing of RIPK1 in vivo in a mouse model of diet-induced obesity dramatically reduces fat mass, total body weight and improves insulin sensitivity by reducing macrophage and promoting invariant natural killer T-cell accumulation in adipose tissue. These findings demonstrate RIPK1 is a genetic driver of obesity in humans, and that reducing RIPK1 expression is a potential novel therapeutic approach to target obesity and related diseases.

All citations are hereby incorporated by reference.

The present invention has been described with regard to one or more embodiments. However, it will be apparent to persons skilled in the art that a number of variations and modifications can be made without departing from the scope of the invention as defined in the claims.

SEQ ID Listings:
SEQ ID NO: 1
CACTTTGTTGCCCAAGCTGGAGTGC[-/AGTG]AGTGGCATGCGATCTCGGCTCACTG
SEQ ID NO: 2
CTCCGGTGctctgtttctgtcccta[A/G]agttcttttcctttccacggagttt
SEQ ID NO: 3
TGTGGAAACACAGAGACACCTTCCC[C/T]AAGCCTCCGCTGTCCAGTTCTGCAC
SEQ ID NO: 4
GTGTTTGTTTGCAGCTCGTTAGCAT[A/C]AAATTTGCAAATTGCTTGGTAGTTT
SEQ ID NO: 5
CTTGATCACCACCATACAGAAAGTA[A/C]CAGAAAAGATGTTTTCTCTTCTTCC
SEQ ID NO: 6
gtgtgatccaccatgcccagccTCA[T/G]CCTTCTTGAATAGCAATGATCACTC
SEQ ID NO: 7
GTGAATTTAACTGCACTGGGTGCCT[-/TA]TATATAAGTGGAATCATACAGTATT
SEQ ID NO: 8
TCCCCTCCCTTGTTTAGCTGGCATC[-/TTTAGAAAGTA]TTTTGCCGTCTAGATT
GGCCTTGTC
SEQ ID NO: 9-NC_000006.12:3063841-3115192 (NCBI ascension number)
>gnl|dbSNP|rs1421085|allelePos=501|totalLen=1001|taxid=9606|
snpclass=1|alleles=′C/T′|mol=Genomic|build=151
SEQ ID NO: 10
ATTTACTGAG ATAAGAAAAC TGAGGACGAA GCAGGTCTGG
GGTGAATCAG GTGCTATCCC TTTGGACAGT CACATGACTG GTGTCTGTTC
AGCACCCAAG GGACCATCAA AGAGGCTGTT GTGGAGAGGG AATCCGAAGG
TCAGGGCCAG AGATAGAAAT CTGGGGAGTC ATCCACTATA TAGGTGATGG
GTTAAAGCCT TAGAACTCTT CTTTTCATGA ATCATTGTTG TTCTTTAGGT
TGTAATGAAG TTTTAGGCCT CAGCTTCCCT GAACTGGAGT GTTTTTCCTT
CACCTTTTCC GGTCTCTGGG TTGCATCGCC AGACTGTCTC TAAGCCCAAC
AAACGCGTTC CTTCCAGGCA AAAGCAGGAG ATGACACACA CCATGAGCCA
GATTTTCCAT GGCAGACTTG TAAGGAACAA GATAATCTCA TTGTTCCTCC
TGCTACTTAA AATAAAGGTA ATATTGATTT TATAGTAGCA GTTCAGGTCC
TAAGGCATGA Y ATTGATTAAG TGTCTGATGA GAATTTGTAG GGTAGTCTCC
CAGACCTGCA GCTACAGGGC ATCTCCCCAC TGGGCCAGGC CTCTGTGCTG
ACCTCCACTG TTATAAGTGG TGTTTTTCTT AGGAATCCTT AGCCCTGTTT
AGTCTACGCA CTGTCCAGAA GCATTTATTT AACCTGATGG ATGGCTCAAT
CCAACCCATC ACCTTAATCT GACCTTAAAG ATGGGAACCT AATGTTTTTG
TTCTTAGTAA ATGTGAAGAC TAATTTTGAC ACTGTTTAGA ATTATTTTCT
TATTAGTAGC AGTAGATTTC ATGAATTTTC TGCTGTGAAA TTTCCTATTA
AAATACTATA ATCTAATGTT AAATGACGAG TTAATGGGTG CAGCACACCA
GCATGGCACA TGTATACATA TGTAACTAAC CTGCACGTTG TGCACATGTA
CCCTAAAACT TAAAGTATAA TAAAAAAAAT AAAAAATAAA TAAAATACTA
TAATCTGACT
>gnl|dbSNP|rs1121980|allelePos=501|totalLen=1001|taxid=9606|
snpclass=1|alleles=′C/G/T′|mol=Genomic|build=151
SEQ ID NO: 11
AAAAACTCTG TGCCTAAGTG ATCAAAGGAA TAAACAAATG
AGAAAGAAAA CAGACTATTA AAAGCAGGCA AAAGGATAGC AGAGCTGAAA
AGTTAGTAAA GGACAAGACA CTCTGACATC AGTGATTAGA CCTTTTGGCT
AAAATTCAGT TCCAGCAGGG GGAAAATGAA AGATAAGCCT GGAATGATAG
ATTGCACCCT GATTATGGAA GGTCCTGAAA GTCAAGCTAA GGAGTCTGGA
TTCTATCCTG CATGTAATGA GGAGCCTCAG AAAGCTTTTG AGAAGGACAG
GAGCACAGTG GAAGGATGTT TGTTATTTGT TGTTCTGATT TTTAAACACC
AATCTACTTA TAATATGGTT ACGGTGAAAG AAACCAAAAG CCAGATAAGG
AGACTACTGC AAAAGCCCAG GCAGACAGGA GCTGGACCTG AACAAGGAGA
CAGCAATGGA AATGAAAAGG AATCAAGAGT TACAGGTAGG CAGGTGGATC
TGAAATCTCA B ATAGTACCAA GACACGTGAC TAGGAAAGGT GGGGCCATAT
AAGAAATAAG GAAGTCAATG TCATTAACAC ATCTACTGAG GGCCCACTAA
GCTAGTTGCT GTGCTGGACA GAAAATGCAA TATGAGTTGG AGGTAAAGAT
ATTTTATTTT AAACATGCTG GGGGAAGAGG TAGATATGGT GGCACTCAGC
ACCCTTCCAA TCTCCTCTTA TTGTGCCTTC CTATGGTGCA GAGGATGAAA
AGGTAAACAC TATTATTTCT CAGACCCTTT TGCAGCTAGG AGTTCTGGAT
GAGAAATAAG CTTCACCAAT TTAATGGCGG CATGTGAGAA TCAGAAGTCA
AAAATAAAAT GGAGGCCATC ACCTTGCTGT TTCAACTTTT TCTGCAGGCA
ACATAGTCAT GAGGCAATAA CATTCCAGAA TCTGGCCATT CACTTTGTAG
TTCTCAAATG CCAGTTGTAA TGGTGGTACC TTCCTGATCC CAACTACAAC
TATGGTTGTG
>gnl|dbSNP|rs17817449|allelePos=501|totalLen=1001|taxid=9606|
snpclass=1|alleles=′A/G/T′|mol=Genomic|build=151
SEQ ID NO: 12
TAGGACCTCC TATTTGGGAC ATTAAGGAAA GCCTTCTCTA
AAAAAAGAAC CCATTTTGAG CAACATGCGA AAATTCTTTG AGTCAGTTGG
CCTCATTTGT AAGCCTTTGA TTGCTTTATT TGTTTGTTTG CTTGCTTGTT
TATTTATTTA TATTTGGAAG GCAATCTTTC TGCTTCCTGA CTGGCTGTCA
GATCTCCCAA ATAACATCAT CCCTTTGCTG GTGTGAATAT AGCCTAGACT
TGAGGCAGCA ATTAACTGAT CATGTAATAG AAGAATCCCA ATCTTCCAGT
TTCTTTTTTT AACTTCTTGG GTGGTTGGGG CACATCATTG CAACAATTAT
TACATTTACT CAAGAGTTTG TCTTTTCTAT TAAAAAAAAA ATTCCATCTA
AGTGCCTTAC GGTGAAGAGG AGGAGATTGT GTAACTGGAG TCTCCCCTTA
ACTGGTCTTT ATTGTTAAAG AAGAGTGATC CCTTTGTGTT TCAGCTTGGC
ACACAGAAAC D GTTTTAATTT AACAGTCCAG CTCCTTTAAT AGATCAATTC
TCTATTGTGG TTTGAATTTG GTGCACTCCC AATTTACTCT AAACTTCTAC
GGGCTTCCTT GGAGAAACTG GGGCAGAGAT GCCGAAGACT CATTTCGGGT
ACAATTTAGA GTTTGTTTAT TTATATAAAT GTAAGGGGTA CCAGTGCAGT
TTTGTTACAT GGATATATTG TGTAGTGGTA AAGTCTGGGT CAGCTTTTAG
TGTAACTCAT CTGAATAATG CACATTATAT CCATTAATTT ATAGTTTTAT
CTCAAACCTC TTATTAATTA ATGCTAGCCA TGGAAGCTAG CTAGCGCTTG
TAAATGCCTG CTGTGTGTTA GATGCTCTAG TAGGTGTTTT AGACACCCTC
ATCTAATCTT CATGGTGATT TTCTGATATA TCCTGAACCT GTACAATTTT
CTCCACTTCT GTCACTACTA CTACAGGCCA AGCCACCATC ATTTCTTGCA
CAGACTTCTG
>gnl|dbSNP|rs9939609|allelePos=501|totalLen=1001|taxid=9606|
snpclass=1|alleles=′A/T′|mol=Genomic|build=151
SEQ ID NO: 13
TGTGTTAGGC GAGATATGGT GAGTGGTTTC AGAGGCTTGT
GTGAGAAAGT TGATACACTG CCCCTACCCC ATGCAAACAC ACACTTTCTT
TTCTTATGAT GACCTTTCTT TAAAAGCATT TGTTGGAATA TGAGATTATA
GGCCATAAGT GAGAAAACAA CTCTTTTTGA GCTGTGAGGA ATACTAGGAG
AGGAGAAAGT GAGCTGTGTG TGCTGCCCAT GGTGGTACGC TGCTATGGTT
CTACAGTTCC AGTCATTTTT GACAGCATGG ATTCAATGCA AAATGGCAAC
ACACACTCTG TATCTTTTGG CAGATCAGAA CATAATGAAA ATAAAATAAA
AAAATTCAAA ACTGGCTCTT GAATGAAATA GGATTCAGAA GAGATGATCT
CAAATCTACT TTATGAGATA ATGTCCTTTT TAAAAATAAA CACTAACATC
AGTTATGCAT TTAGAATGTC TGAATTATTA TTCTAGGTTC CTTGCGACTG
CTGTGAATTT W GTGATGCACT TGGATAGTCT CTGTTACTCT AAAGTTTTAA
TAGGTAACAG TCAGAAATGG AGTGGGAGAG CATAAAAGCA AACTGAAATG
CAAATAGCTG GTACCCTGAA GCCATTAACT TTAAGCTGGT TATTCCTGAC
CTACTGTTTG GACATAAGAT GGTAGAGAGG CTGAGTGTGA CTTGAACATT
TGTTCCTTAG AAACACCATC CTTGGGCTGG GCGCAGTGGC TCACGCCTGT
GTTTCCAGGA CTTTGGGAGG CTGAGGCGGG CAGATCATGA GGTCAATAGA
TCGAAACTAT CCTGGCTAAC ACGGTGAAAT CCCATCTCTA CTAAAAATAC
AAAAAATTAG CCAGGTGTGG TGGCGGGTGC CTGTAGTCCC AGCTACTCGG
GAGGCTGAGG CAGGAGAATT GCTTGAACCC AGGAGGCAGA GGTTGCAGTG
AGCCAAGGTC GCACCACTGC ACTCTAGCCT GGGCAACAGA GTGAGACTCC
TTCTCAAAAA
SEQ ID NO: 14
TGGGAATGCAGCCCAAG
SEQ ID NO: 15
CCTTACGGTACTTGTTGACTATCG
SEQ ID NO: 16
AGACTGCTCGTCAAGTGTGG
SEQ ID NO: 17
TTGAGCTGTAGCCTGAACCTTA
SEQ ID NO: 18
GATTACGCGTTGCAGTTCCAACTTTGCTC
SEQ ID NO: 19
GATTAAGCTTTGGCTTCTTCAAGTGTAAG
SEQ ID NO: 20
gtaaaatcgataaggatccgGGCAGTAAGTACAGTCCC
SEQ ID NO: 21
aaggctctcaagggcatcggTCAAAGCCACCTAAGTGC
SEQ ID NO: 22
taTATATAAGTGGAATCATACAGTATTTGTCC
SEQ ID NO: 23
AGGCACCCAGTGCAGTTA
SEQ ID NO: 24
GTCCCATAGTACATTCACTTTCC
SEQ ID NO: 25
AAGCCAGACACAAAAGGACAA
>gnl|dbSNP|rs67432438|allelePos=501|totalLen=1001|taxid=9606|
snpclass=2|alleles=′-/AGTG′|mol=Genomic|build=151
SEQ ID NO: 26
CTTTTTCTGA CACAGATTGG TTTTGACTTC TGAACCGTCA
TATAAATAGA ATTACATAGT ATGTATGCTT TTGTATAAGG CGACTTTCAC
TTATCACGTT TTTGCATTTC GTTCATGCGG TTTTCATTCA TGTGTATCAG
TAGTTCATTT TTATTGCTGA GTAGTATCCC ATCGTGTTCA TACACCAGTG
TGTTCACTCA TTCTCTTACT GATGGGCAGC TGTTTCCAGT TTGGAGCTAT
TATAAATAAA GTTGCTATGA ACATTTTACA AGTGTTTTCC AACATACGTT
TTCATTTCTC TTGGGATTGG AATTGCTGGG TCATAGTGCA GGTGTTTGTG
GTTTTATAAA CAAAATAAAT GCCTATGAAA TTTCAATGAA TATTTTATGC
CCCAGAAAGA GTATTTTTCA TGGTCTGTAT CTGGATGTAG ATATTTATTA
TTTTATTTTA TTTTAATTTT TTTGAGACAG AGTCTCACTT TGTTGCCCAA
GCTGGAGTGC N AGTGGCATGC GATCTCGGCT CACTGCAACC TCCGCCTCCC
AAATTTAAGC AATTCTCCTG CCTCAGCCTC CTGAGTAGCT GGGATTAGAG
GCGCGTGCCA CCACGCCTGG CTGATTTTTG TATTTTTAGT AGAGACGGGG
TTTCACCATG TTGGCCAGGC TGGTCTTGAA CTCCTGACCT CGTGATCCGC
CCGCCTCAGC CTCCCAAAGT GCTGGGATTA CAGACGTGAG CCACGGCGCC
CAGCCGGGAT GTGGGTATTT CTGGAATACA AAACATCCAT AGCCCTCCTT
TTACTCTCAA AGCAAAACAC AGACACGCAC ATTCACATGC ACACACCAGT
GTGTCCGTGT CCTGTTGGTT TGCATGGAGT TTCTGTTTTG CCACGAAGCT
TTTTGCCAGC CCTTTTCCCA AAGTGTTTGT ACCATTTTAT ACTCCCACCA
GTGATGTTTG AGAGGTCTGG TTGCTTCACA TGTTTGCCAG TGTTTGGTGT
ATTAGTCTTT
>gnl|dbSNP|rs4959774|allelePos=501|totalLen=1001|taxid=9606|
snpclass=1|alleles=′A/C/G′|mol=Genomic|build=151
SEQ ID NO: 27
CACAGTGAAT Tttttctttt gtgttttata attttggaaa gggagctcat
gttaaatttg tatttgtagg aattctgtct ggcctggctt gaggttgTTG
TTTGCTTTAG CCTATAACCT CAGGATGGCA TCAGCCTGTG ACCACTTCCT
GTGTAAAAGT TCATAGTTTA GGCGATCCTC TCAATTCAAA GTCTGTATCT
ATTTCAAGAC AGGCCCAAGG TTATAAATTT GCACAAACtt tttttttttt
tttttaataC CTTGTGCCCA GTCCAAGACA AATATGTTTG TTTGCCATGG
GCTGCTTTTT TTCTAGTTCA TCCTTTCATC AAAGGGTTGC ACATTGAGCA
GTGCCGTGTT AAGGGGGGAT TCTTCATTCT ACCTTCTTTT CTTGTATTCC
TTGACCTCAG CTGGGCTTTA AACCTTGACT TCTGGATGGC GAGGTTGGCA
CTCCCCTGTG TGAGCTACTG CCTGCCTCCG GTGctctgtt tctgtcccta
V agttcttttc ctttccacgg agttttggta tgcatttcaa tgaatgttg
tattatgttg tccaacatac ttgtgtcctt tatagctgga gggttcttaa
aattttctaa tgctccatat tgccagaaat aaaaAGGCTG AGGGAATAAA
TATTATAGTC TTCTCTGATT CTTAAAAATA TGAAAAATAG CTCATCAGAT
GACTGTAAGT CTATCCTCAG AAATCCAAGG CACACCAAGC ATGTGGTGGA
ATGTTAACAA AAGTTTTTAA AAATGATCTT TTCTgccagg tacaatggct
cacacctgta atctcagcac tttgggaggc tgaggtggga ggatcccttg
atcttaggag tttgagacca gcctgggcaa catagtgaga tcccccggcc
cccccatctc tacaaataat taaaaaatca gctggtcacg gtggggtgca
cctgtggtcc cagctactca aggaggctga gggaggagga ttgcctgagc
>gnl|dbSNP|rs2272990|allelePos=501|totalLen=1001|taxid=9606|
snpclass=1|alleles=′A/C/G′|mol=Genomic|build=151
SEQ ID NO: 28
CTCTACTGGT TGCTTTAAAG GGAAGGCTCT CAGAACCTCT
CAGGAATGAA CTGCCTTTTC ATTTTTATAC TTAGGGCTGG TCCTCACTGA
AAATGTGAAG CGAGTGAATA GGTCTGTCTT CACCCATCCT CCTGTTTCCT
TTTCATTGTC CCCCCACCAC ACTTATCTCT TTGTCTTTCT CTATTTCCTT
CTTGTTCATG TCTCTTCTGT TACTTACCAG CTGTGAGAGT CCTTCACTAC
CCAGTCTCAG AGTCCACATC AGCGACAATA ACAATCCGAA GAGCCATCGT
CACCCTGACA TCTTGGCAGG CTCCCTCTCA CTCTACCCAC CAACGGCTCC
GCAGCAGCCA ACGGCTCCAC AACAGCCAAC GTCCCCATCC CGCTCAGAAC
TTAGCCCACC CACCCCTGCT CCCTACTCAC TCAATGCAGT TGGGCCCCTT
GTACACTGTT TTCATGATCA TGAGTCCCTG GGTTCTGTGG AAACACAGAG
ACACCTTCCC V AAGCCTCCGC TGTCCAGTTC TGCACTCTCC AGGAAGTCAC
TGGATTTCAT CTTAATGACA TTCAAGGACA TGTCTGGTTG CATTCTGAAG
CTCAAGAACG CCCAAAATGG TACCACTTTT TCCCCCCCCG GCAGAGCTGT
ACCCTGTAAA GAAAACAGAG AGAAAACCTC AGGGCAAGAC TATATATATA
TATATATATA TATATATGTT TTTTTTTTTC TCCTTTGGAG ACAGGGTCTC
ACTTTGTCAG TCAGGCTGGA GTGCAGTGGC ACAATCATAG CTCACTGGAG
CCTCGACCTC CTGGGCTCAG GCAATCCTCC TCCCTCAGCC TCCTTGAGTA
GCTGGGACCA CAGGTGCACC CCACCGTGAC CAGCTGATTT TTTAATTATT
TGTAGAGATG GGGGGGCCGG GGGATCTCAC TATGTTGCCC AGGCTGGTCT
CAAACTCCTA AGATCAAGGG ATCCTCCCAC CTCAGCCTCC CAAAGTGCTG
AGATTACAGG
>gnl|dbSNP|rs6907943|allelePos=501|totalLen=1001|taxid=9606|
snpclass=1|alleles=′A/C′|mol=Genomic|build=151
SEQ ID NO: 29
TTCATTGATT CATTCATTCC TTCATTGATT TACATTTTTG
GAGCATGCAC TGCCAGTTAC TGGGCAGGAG TACTGTGAGG AAGTGAGAAA
CCGCTCTGCC TTCTAACGCT TCTGGCCTGT GCCTGGAAGA GATCGGTACA
GACCCAAGCA TGACCCCAGC ACGTGGGAGT GATGTGTTGG AGGTGTGCAC
CAGGCTCTTG AGGCGCTGGC TCTGCCAGCC TCAGCATAGC ACCTTTCCTG
CCCACAGGCA CAACGAGGCC CTCTTGGAGG AGGCGAAGAT GATGAACAGA
CTGAGACACA GCCGGGTGGT GAAGCTCCTG GGCGTCATCA TAGAGGAAGG
GAAGTACTCC CTGGTGATGG AGTACATGGA GAAGGGCAAC CTGATGCACG
TGCTGAAAGC CGAGGTAGAG AGGGCCCCTC CGCACGGGGA TCCCCAGCGC
TTGGGCCCTG GCTGTCTGTT ATGGCTCCCC TTGGGGTGTT TGTTTGCAGC
TCGTTAGCAT M AAATTTGCAA ATTGCTTGGT AGTTTGAATT TTCTTTTCCT
TTTTTTATAG AGACAAGGTC TTGCTATGTT GCCCAGGCTG AACTCCAACA
CCTGGTCTCA AGTGATCCTC CCACCTCAGC CTCTCAAGTA GCTGGGATTA
CAGGCTGGAT TTCCTGAAGT CAGTTTCTCC TTAAAACATC TATGCCTTGA
TTGGGCTCCG TGGGTCACTT CTGTAATCTC AGCACTTTTG GCAGGAGGAT
CACTTGAGCC TTGGTGCTGA AGACCAGCCT GGATAACACA GTGAAATCCC
CTCTCTACAA AAAATAAAAG GTGCATGTGA ACAGAGCAAG ACCCTGTCTC
TGAAAAAAAG AGAAAATCTT CCTTGCCTTT TACTGCCCTG AGAGTGGTCT
CTTCATCCTC TCGATCCTGT GGACAGTCCT CCATAGTCTG TGTCTTAGGA
ATGTGATTGG CTGTTTACCA TACCTGTCCG CTGCTCTGCA ATCTACAGTT
CAGAGACAAG
>gnl|dbSNP|rs2064310|allelePos=501|totalLen=1001|taxid=9606|
snpclass=1|alleles=′A/C/G/T′|mol=Genomic|build=151
SEQ ID NO: 30
CACACGACAT CATACTAAGT TTACATTAAG GACATGAACA
ATGCCATGCA AATGTCGCGT GTAAAATAAC ATTAAGAGAA AAAAGAATAC
AAGGGAACAT GTTCACACTG ATTACAATCA TAGAAAAATC TGAGATAAAC
ATGATGTTAA AACTACGCTT AAGTGAATTA ATATCTATTT GATGACCTGG
TAGCAGATAC TTCAGGCTTC TGTAGAGTCT CAGGTCACTG GTTCTTATAA
TTGTATTCTT GTTATGTGAG CATCAATACT AACAGAAACA ATTCCAGTTT
CTATTATAGT GGTTAAATTA CAGTTTTGCT TAACAATAAA AAATCAGTAT
ACAAACAAGA TGTTTCCTAA AAATGTATAT GTATTTTGGT CGTTGGTTAA
CAGGAAAAAA ATTTTAATGA TAGAAGTGAG ATTCCCCCTC AAACCATATT
TATGACATAA CGTAGCAGAG CTACACTGTT AATAGCTTGA TCACCACCAT
ACAGAAAGTA N CAGAAAAGAT GTTTTCTCTT CTTCCTTGGT ATACCTTTGG
ACTTAGGAAA AACTTCTGAG TAAGCAAAGT TGGTCCCCCT CTCTCCTCTC
TTTCTGCTTC TCTCTCAGGT CCTCCTGATG CCCTGACTGA GTCCAAACAG
GTGCACCTGA ACTTTCCCTC ATCTTGCACA ATGCCTCATG TCCCCAATGA
TTTCTCTCTC ATCTCAGAAG CTAAGCAGGG TTGGGCCTGG TTAGTACTTG
GATGGGAGAC CAAAGACTTA TTTCCCAATC AAAATGTTTA CTTCAAACAG
CTGATCTTCT CTAGTTCTGG GGTGGAGAGC AATCTGATCA AACTATAATA
CCATGCGTAT AAGTGATCAC CTCTTCTCCT TCCCAAGGGT ATTTTTGTGA
GATGTCTGTT GGCTTGGCAA TTGTGGGGTG GGAGAAGAAA GTGGAAAAGT
GGGAGTATTT GCCATAAAGG CAGCCATATC CCGCCACCCC TCCCCATACC
CTGTGCAATC
>gnl|dbSNP|rs7753662|allelePos=1146|totalLen=1346|taxid=9606|
snpclass=1|alleles=′G/T′|mol=Genomic|build=151
SEQ ID NO: 31
CTTCTTGGGT GAtcatcagc tcccatggtt tgatgatcat ctctttgcaa
atgcttctta aatctgtgtc tctggctctg gaatggatat ccacttgtct
tacagtcaga tccatcgcag gtaccagagg aacctccaaa gcagcatccc
ccaaacacat cctgtcgaca ctgtcctgca gctcgccccg cttcacccct
cttcatatgc ttgggaagtg gcatcgccat ccttttacca agttgttgcc
caataagaaa cttcagagtc atcccagact cgcccttgcc cctctctccc
acattctatc agatctcaaa acctgtgaat tcaagatata tgtatgactc
aagatatatc tgcaacccat ctgcctttct ctgtctccac tggcctctgc
tcagttcaaa ccctcctcgt ttctgcagca ggctccttcc accgtccacc
ggtcgtccta aaaggcacat gcaaccctct attcatgtgg cagccaaaat
ccctcacata ttggaaatcc ttcccagatg ctctgtggtc ttcaggatga
agttcgaatt cctcagcgtg gaataaaagg ccttcatgtt ctggcctttg
cttctctact ggcctcgtcc ctaaccctcc cttctttgtc agtgtgcagt
tacactgAGG AATTTGTGGG ATGCGTCATG ATCTCTGTAA CTTCTAGTCT
GTTGATATCC TGCTTCTCTT CCTGGAGTCC TCCTCCTTAA GTCTCCCTTT
CCCATTCTTT ACCTGCTAAT TTTTCTGGAC TCAACAAAGC CTTCCTGGAC
CTTTTTAGTC AGGTTTGTGC TACTGGAACC CCTTGTACAT CCtttttttt
ttttttttcc cgtgagacag agtctcattc tgtcgccagg ttggagtgca
gtggtgcagt ctcggctcac tgcagcctcc acctcccagg ttcaagcaat
tcttctgcct cagcctcccg agtagctggg actacaggtg cacaccacca
tgcccagcta atttttatat ttttagtaga gacggggttt caccatgttg
gccaggatgg tcttgatctc ttgacgtcat gatccgcctg tctcaacctc
ccaaagtgct gggattacag gtgtgatcca ccatgcccag ccTCA K
CCTTCTTGAA TAGCAATGAT CACTCTGTGT TTTAAGTCAC TCTCATTAAA
CTGAGCTCCT AAAGACAAGG ACTGCCCTGT ATTTGCTTTT GCACATGGAG
CATGCCATTG GCCTTGCCCA CATAATCTTT TTGTTCCATC AGCAGGCAGC
TTTGTCGAGA ATTTTGTTTT ATCTGCAGAC ATTAAGATAT TGTTTGGTCA
>gnl|dbSNP|rs5873855|allelePos=501|totalLen=1001|taxid=9606|
snpclass=2|alleles=′-/TA′|mol=Genomic|build=151
SEQ ID NO: 32
GATGACATCA CTGAGTACTG CCCAAGAGAA ATTATCAGTC
TCATGAAGCT CTGCTGGGAA GCGAATCCGG AAGCTCGGCC GACATTTCCT
GGTAAGAGCA TCTTTTCTGA CTGTGTAGGA TGCATCCTGT GTGGTGATTT
TCCTAGTAAC ATATTGTTAG GAACTTGGTT TGAATCCTCA GAAAACTGAG
TTCGACTTGA CTTGTGGTTT TAAACTGATT TTCTAGATTT CAAACATGTT
ATTCAGTGCA AAGAAGCCCT CTCTCTCAAA AGCATGATGG CACATAAGGC
CTTCTTGTTT CCTTTTTTTG TAATTAACTT TTTAACTTGG GTTTAAATAT
ACGTAACATG AAATTTAACA TTGTAACTGT TTTCAAGTGC ACTGTTCAGG
GGCAGTAAGT ACAGTCCCAT AGTACATTCA CTTTCCATGG TCTCTCTCCC
TGCCCCTGGC AGCCCCTATC CTACTTTCTG TCTCTGTGAA TTTAACTGCA
CTGGGTGCCT N TATATAAGTG GAATCATACA GTATTTGTCC TTTTGTGTCT
GGCTTACTCC ACTTGGCATG ATGTCCTCGA GGTTCATCTG TGTTGTAGCA
TATGTCAGAA TTTCTTTCCT TTTCAAGGCT GAATAATATT CCATTGTATG
AATGGAGTGC ATTTTGTTTA TCCATTCGTC CGTCTATGGG CACTTAGGTG
GCTTTGACCA GCCTTCTTGC TTCTTCACTT CTTTTATTTC CATCCCCTCC
CTTGTTTAGC TGGCATCTTT AGAAAGTATT TTGCCGTCTA GATTGGCCTT
GTCATACAGG GATGAGGAAA GGTGCCGGGA TGTTTGCTGA GCAGTATTCT
ATGGTGTGGA TGTACCACGG TTTGGTTAAC TACTCACTCC TTGAAAGACT
TGTTTCCAGT TCTTAACTGT TGGGACTAAT GCAGCTGTAA ATATTCGTGG
ACATGTTTTT GTGTGAATAT AAGTCTTCAT TTCTCTGGAG TAATACCCAG
GGGTACAATA
>gnl|dbSNP|rs141325626|allelePos=501|totalLen=1001|taxid=9606|
snpclass=2|alleles=′-/TTTAGAAAGTA′|mol=Genomic|build=151
SEQ ID NO: 33
TCTCTCTCAA AAGCATGATG GCACATAAGG CCTTCTTGTT
TCCTTTTTTT GTAATTAACT TTTTAACTTG GGTTTAAATA TACGTAACAT
GAAATTTAAC ATTGTAACTG TTTTCAAGTG CACTGTTCAG GGGCAGTAAG
TACAGTCCCA TAGTACATTC ACTTTCCATC GTCTCTCTCC CTGCCCCTGG
CAGCCCCTAT CCTACTTTCT GTCTCTGTGA ATTTAACTGC ACTGGGTGCC
TTATATATAA GTGGAATCAT ACAGTATTTG TCCTTTTGTG TCTGGCTTAC
TCCACTTGGC ATGATGTCCT CGAGGTTCAT CTGTGTTGTA GCATATGTCA
GAATTTCTTT CCTTTTCAAG GCTGAATAAT ATTCCATTGT ATGAATGGAG
TGCATTTTGT TTATCCATTC GTCCGTCTAT GGGCACTTAG GTGGCTTTGA
CCAGCCTTCT TGCTTCTTCA CTTCTTTTAT TTCCATCCCC TCCCTTGTTT
AGCTGGCATC N TTTTGCCGTC TAGATTGGCC TTGTCATACA GGGATGAGGA
AAGGTGCCGG GATGTTTGCT GAGCAGTATT CTATGGTGTG GATGTACCAC
GGTTTGGTTA ACTACTCACT CCTTGAAAGA CTTGTTTCCA GTTCTTAACT
GTTGGGACTA ATGCAGCTGT AAATATTCGT GGACATGTTT TTGTGTGAAT
ATAAGTCTTC ATTTCTCTGG AGTAATACCC AGGGGTACAA TAGCTAGGTT
GTATGGTAGT TTCATGTACA GTTTATTTTA AAAATTCCCT AACATTTTCC
CAGAGGGGCT GTGCCATTTT ATGTTCGGTG TCCGAGTGAT GCAGGTTCTT
GTATCCTCAC CAGCATGGGG TACCCAGGCT CCTCACACTT CTGCTTAGTC
AGCTACAAAT TTATTTGAGT ATTCCCACAA GCCCCACTTC AGGTTTGGTA
ATTCGTTAGA AAGACTCACA TAACTCAAGA AAACATTTTA CTTACGTCTC
CCCTTTTACT

### REFERENCES

1. Berg, A. H. & Scherer, P. E. Adipose tissue, inflammation, and cardiovascular disease. Circ Res 96, 939-949 (2005).
2. Weisberg, S. P., et al. Obesity is associated with macrophage accumulation in adipose tissue. The Journal of clinical investigation 112, 1796-1808 (2003).
3. Stunkard, A. J., Foch, T. T. & Hrubec, Z. A twin study of human obesity. JAMA 256, 51-54 (1986).
4. Frayling, T. M., et al. A common variant in the FTO gene is associated with body mass index and predisposes to childhood and adult obesity. Science 316, 889-894 (2007).
5. Gerken, T., et al. The obesity-associated FTO gene encodes a 2-oxoglutarate-dependent nucleic acid demethylase. Science 318, 1469-1472 (2007).
6. Scuteri, A., et al. Genome-wide association scan shows genetic variants in the FTO gene are associated with obesity-related traits. PLoS Genet 3, e115 (2007).
7. Dina, C., et al. Variation in FTO contributes to childhood obesity and severe adult obesity. Nat Genet 39, 724-726 (2007).
8. Speakman, J. R., Rance, K. A. & Johnstone, A. M. Polymorphisms of the FTO gene are associated with variation in energy intake, but not energy expenditure. Obesity (Silver Spring) 16, 1961-1965 (2008).
9. Smemo, S., et al. Obesity-associated variants within FTO form long-range functional connections with IRX3. Nature 507, 371-375 (2014).
10. Loos, R. J., et al. Common variants near MC4R are associated with fat mass, weight and risk of obesity. Nat Genet 40, 768-775 (2008).
11. Thorleifsson, G., et al. Genome-wide association yields new sequence variants at seven loci that associate with measures of obesity. Nat Genet 41, 18-24 (2009).
12. Karunakaran, D., et al. Targeting macrophage necroptosis for therapeutic and diagnostic interventions in atherosclerosis. Sci Adv 2, e1600224 (2016).
13. Silke, J., Rickard, J. A. & Gerlic, M. The diverse role of RIP kinases in necroptosis and inflammation. Nature immunology 16, 689-697 (2015).
14. Kondylis, V., Kumari, S., Vlantis, K. & Pasparakis, M. The interplay of IKK, NF-kappaB and RIPK1 signaling in the regulation of cell death, tissue homeostasis and inflammation. Immunol Rev 277, 113-127 (2017).
15. Davies, R. W., et al. A 680 kb duplication at the FTO locus in a kindred with obesity and a distinct body fat distribution. Eur J Hum Genet 21, 1417-1422 (2013).
16. Speakman, J. R. The ‘Fat Mass and Obesity Related’ (FTO) gene: Mechanisms of Impact on Obesity and Energy Balance. Curr Obes Rep 4, 73-91 (2015).
17. Civelek, M., et al. Genetic Regulation of Adipose Gene Expression and Cardio-Metabolic Traits. Am J Hum Genet 100, 428-443 (2017).
18. Laakso, M., et al. The Metabolic Syndrome in Men study: a resource for studies of metabolic and cardiovascular diseases. J Lipid Res 58, 481-493 (2017).
19. Consortium, G. T. Human genomics. The Genotype-Tissue Expression (GTEx) pilot analysis: multitissue gene regulation in humans. Science 348, 648-660 (2015).
20. van Nas, A., et al. The systems genetics resource: a web application to mine global data for complex disease traits. Front Genet 4, 84 (2013).
21. Cowell, I. G. E4BP4/NFIL3, a PAR-related bZIP factor with many roles. Bioessays 24, 1023-1029 (2002).
22. Lynch, L., et al. Regulatory iNKT cells lack expression of the transcription factor PLZF and control the homeostasis of T(reg) cells and macrophages in adipose tissue. Nature immunology 16, 85-95 (2015).
23. Takahashi, S., et al. A promoter in the novel exon of hPPARgamma directs the circadian expression of PPARgamma. J Atheroscler Thromb 17, 73-83 (2010).
24. Lynch, L., et al. Adipose tissue invariant NKT cells protect against diet-induced obesity and metabolic disorder through regulatory cytokine production. Immunity 37, 574-587 (2012).
25. Ji, Y., et al. Activation of natural killer T cells promotes M2 Macrophage polarization in adipose tissue and improves systemic glucose tolerance via interleukin-4 (IL-4)/STAT6 protein signaling axis in obesity. J Biol Chem 287, 13561-13571 (2012).
26. Berger, S. B., et al. Cutting Edge: RIP1 kinase activity is dispensable for normal development but is a key regulator of inflammation in SHARPIN-deficient mice. J Immunol 192, 5476-5480 (2014).
27. van der Klaauw, A. A. & Farooqi, I. S. The hunger genes: pathways to obesity. Cell 161, 119-132 (2015).
28. Vucic, D., Dixit, V. M. & Wertz, I. E. Ubiquitylation in apoptosis: a post-translational modification at the edge of life and death. Nature reviews. Molecular cell biology 12, 439-452 (2011).
29. Chan, F. K., Luz, N. F. & Moriwaki, K. Programmed necrosis in the cross talk of cell death and inflammation. Annu Rev Immunol 33, 79-106 (2015).
30. Peltzer, N., Darding, M. & Walczak, H. Holding RIPK1 on the Ubiquitin Leash in TNFR1 Signaling. Trends Cell Biol 26, 445-461 (2016).
31. Roderick, J. E., et al. Hematopoietic RIPK1 deficiency results in bone marrow failure caused by apoptosis and RIPK3-mediated necroptosis. Proc Natl Acad Sci USA 111, 14436-14441 (2014).
32. Gautheron, J., et al. The necroptosis-inducing kinase RIPK3 dampens adipose tissue inflammation and glucose intolerance. Nat Commun 7, 11869 (2016).
33. Dannappel, M., et al. RIPK1 maintains epithelial homeostasis by inhibiting apoptosis and necroptosis. Nature 513, 90-94 (2014).
34. Berzins, S. P., Smyth, M. J. & Baxter, A. G. Presumed guilty: natural killer T cell defects and human disease. Nat Rev Immunol 11, 131-142 (2011).
35. Lynch, L. Adipose invariant natural killer T cells. Immunology 142, 337-346 (2014).
36. Huh, J. Y., et al. Deletion of CD1d in Adipocytes Aggravates Adipose Tissue Inflammation and Insulin Resistance in Obesity. Diabetes 66, 835-847 (2017).
37. Vieth, J. A., et al. TCRalpha-TCRbeta pairing controls recognition of CD1d and directs the development of adipose NKT cells. Nature immunology 18, 36-44 (2017).
38. Mookerjee-Basu, J. & Kappes, D. J. iNKT cells do a fat lot of good. Nature immunology 18, 10-12 (2016).
39. Lynch, L., et al. Invariant NKT cells and CD1d(+) cells amass in human omentum and are depleted in patients with cancer and obesity. European journal of immunology 39, 1893-1901 (2009).
40. Engel, I., et al. Innate-like functions of natural killer T cell subsets result from highly divergent gene programs. Nature immunology 17, 728-739 (2016).
41. Harris, P. A., et al. Discovery of a First-in-Class Receptor Interacting Protein 1 (RIP1) Kinase Specific Clinical Candidate (GSK2982772) for the Treatment of Inflammatory Diseases. J Med Chem 60, 1247-1261 (2017).
42. Ramkhelawon, B., et al. Netrin-1 promotes adipose tissue macrophage retention and insulin resistance in obesity. Nature medicine 20, 377-384 (2014).
43. Carvalho, B. S. & Irizarry, R. A. A framework for oligonucleotide microarray preprocessing. Bioinformatics 26, 2363-2367 (2010).
44. Ritchie, M. E., et al. limma powers differential expression analyses for RNA-sequencing and microarray studies. Nucleic Acids Res 43, e47 (2015).
45. Karunakaran, D., et al. Therapeutic Inhibition of miR-33 Promotes Fatty Acid Oxidation but Does Not Ameliorate Metabolic Dysfunction in Diet-Induced Obesity. Arterioscler Thromb Vasc Biol 35, 2536-2543 (2015).

What if claimed is:

1. An isolated nucleic acid associated with increased risk of weight gain, obesity, inflammation, diabetes or combination thereof, the nucleic acid comprising:
a) at least 7 consecutive nucleotides of SEQ ID NO: 1 and comprising “CAGTC” at position 26, or a sequence complementary thereto;
b) at least 7 consecutive nucleotides of SEQ ID NO: 2 and comprising G at position 27, or a nucleotide sequence that is complementary thereto;
c) at least 7 consecutive nucleotides of SEQ ID NO: 3 and comprising T at position 26, or a sequence complementary thereto;
d) at least 7 consecutive nucleotides of SEQ ID NO: 4 and comprising C at position 26, or a sequence complementary thereto;
e) at least 7 consecutive nucleotides of SEQ ID NO: 5 and comprising C at position 26, or a sequence complementary thereto;
f) at least 7 consecutive nucleotides of SEQ ID NO: 6 and comprising G at position 26, or a sequence complementary thereto;
g) at least 7 consecutive nucleotides of SEQ ID NO: 7 and comprising “TTA” at position 26, or a sequence complementary thereto;
h) at least 7 consecutive nucleotides of SEQ ID NO: 8 and further comprising TTTAGAAAGTA at position 26, or a sequence complementary thereto; or
i) at least 70% identity to the nucleotide sequence defined in any one of a)-h).

2. The nucleic acid of claim 1, which is labeled.

3. A nucleic acid that hybridizes to the nucleic acid defined in claim 1 or its complement under stringent hybridization conditions.

4. The nucleic acid of claim 1 for reducing the expression of RIPK1 in a cell.

5. The nucleotide sequence of claim 4, wherein said nucleotide sequence comprises a double-stranded nucleic acid that comprises:
a sense strand; and
an antisense strand comprising a region that is substantially complementary to the sequence listed in any one of SEQ ID NOS: 1-8, wherein said region of complementarity is at least 7 contiguous nucleotides in length and wherein said nucleic acid, upon contact with a cell expressing said RIPK1 gene, reduces expression of said RIPK1 gene.

6. The nucleotide sequence of claim 5, in which the double-stranded nucleic acid comprises a sequence that is substantially complementary to SEQ ID NO: 4.

7. The nucleic acid of claim 1, for use in an assay to detect or identify a subject that has or is at risk of developing diabetes, obesity, inflammation or any combination thereof.

8. The nucleic acid of claim 7, wherein the obesity is diet-induced obesity, the inflammation is adipocyte or liver tissue inflammation, or any combination thereof.

9. The nucleic acid of defined by claim 1 for treating, reducing or preventing weight gain, obesity, diabetes or inflammation, or ameliorating a condition associated therewith in a subject in need thereof.

10. The nucleic acid defined by claim 9, wherein the condition comprises one or more of diet-induced weight gain, diet-induced obesity, fat mass, liver inflammation, adipose tissue inflammation, adipose size, adipose macrophage accumulation, lipid accumulation, increasing the number of invariant natural killer T-cells (iNKT cells) in adipose tissue, improving glucose tolerance, insulin sensitivity, glucose homeostasis, fasted blood glucose, plasma insulin levels, response to both glucose and insulin challenge or any combination thereof.

11. A method of detecting or screening a subject for a RIPK1-associated nucleotide sequence or protein associated with weight gain, obesity, inflammation, diabetes, or a combination thereof, and treating said subject, the method comprising identifying the presence or absence the nucleotide sequence defined by claim 1 in a biological sample from the subject, the sample comprising genomic DNA, mRNA or protein from the subject, wherein the presence of the nucleotide sequence is indicative that the subject has or is at risk for weight gain, obesity, inflammation, diabetes, or a combination or is a carrier for one or more genes associated weight gain, obesity, inflammation, diabetes, or a combination thereof, and treating the subject.

12. The method of claim 11, wherein the one or more polymorphisms in the ripk1 gene are relative to:
a) rs67432438
(SEQ ID NO: 1)
CACTTTGTTGCCCAAGCTGGAGTGC[-/AGTG]AGTGGCATGCGATCTC
GGCTCACTG;
b) rs4959774
(SEQ ID NO: 2)
CTCCGGTGctctgtttctgtcccta[A/G]agttcttttcctttccacg
gagttt;
c) rs2272990
(SEQ ID NO: 3)
TGTGGAAACACAGAGACACCTTCCC[C/T]AAGCCTCCGCTGTCCAGTT
CTGCAC,
d) rs6907943
(SEQ ID NO: 4)
GTGTTTGTTTGCAGCTCGTTAGCAT[A/C]AAATTTGCAAATTGCTTGG
TAGTTT,
e) rs2064310
(SEQ ID NO: 5)
CTTGATCACCACCATACAGAAAGTA[A/C]CAGAAAAGATGTTTTCTCT
TCTTCC,
f) rs7753662
(SEQ ID NO: 6)
gtgtgatccaccatgcccagccTCA[T/G]CCTTCTTGAATAGCAATGA
TCACTC,
g) rs5873855
(SEQ ID NO: 7)
GTGAATTTAACTGCACTGGGTGCCT[-/TA]TATATAAGTGGAATCATA
CAGTATT,
h) rs141325626
(SEQ ID NO: 8)
TCCCCTCCCTTGTTTAGCTGGCATC[-/TTTAGAAAGTA]TTTTGCCGT
CTAGATTGGCCTTGTC,
or
the complement thereof, and wherein the polymorphic site is in brackets, underlined and in bold and wherein the risk alleles are defined as recited herein.

13. The method of claim 11 wherein the step of detecting is performed by microarray analysis, restriction analysis, probe hybridization, nucleotide sequence amplification, PCR, electrophoretic-based nucleic acid analysis, ELISA, DNA aptamers, molecular barcoding, DNA sequencing, protein sequencing, antibody binding analysis, mass spectrometry, gene chip analysis, a combination thereof or any other method known in the art.

14. The method of claim 11 in which the nucleic acid is genomic DNA or mRNA.

15. The method of claim 14, wherein the genomic DNA or mRNA is obtained from blood.

16. The method of claim 11, wherein the genomic DNA or mRNA is obtained from adipose tissue.

17. The method of claim 11 further comprising, before detecting, a step of selecting a subject that is overweight, obese, or that exhibits one or more signs of diabetes or inflammation.

18. The method of claim 11, in which the polymorphism is a mutation in a promotor sequence upstream of the ripk1 gene.

19. The method of claim 11, further comprising one or more additional steps defined by: administering an inhibitory agent or nucleic acid that reduces expression or activity of RIPK1 in the subject, changing the diet of the subject, increasing the exercise regimen of the subject, administration of one or more additional tests, administration of one or more surgical procedures, gene therapy, monitoring the subject, analyzing the body composition of the subject, counselling the subject, administration of additional therapeutic agents, stapling the stomach of the subject, applying a gastric band, gastric bypass, gastric balloon, and/or gastric sleeve to the subject.

20. A method for assessing risk, diagnosis, prognosis, or any combination, of obesity, diabetes, inflammation, or any combination, in a subject, comprising:
a) detecting, in a nucleic acid sample from said human subject, expression levels of RIPK1, E4BP4, or both; and
b) comparing expression level data obtained in step a) from said human subject to expression levels of either RIPK1, E4BP4, or both, from a healthy or affected control group to make a risk assessment, diagnosis, prognosis or any combination, of either obesity, diabetes, inflammation or any combination, in said human subject.


SEQUENCE LISTING (33 sequences)

SEQ ID NO: 1 - DNA, 54 bp, Homo sapiens
cactttgttgcccaagctggagtgcagtgagtggcatgcgatctcggctcactg

SEQ ID NO: 2 - DNA, 51 bp, Homo sapiens
ctccggtgctctgtttctgtccctagagttcttttcctttccacggagttt

SEQ ID NO: 3 - DNA, 51 bp, Homo sapiens
tgtggaaacacagagacaccttccctaagcctccgctgtccagttctgcac

SEQ ID NO: 4 - DNA, 51 bp, Homo sapiens
gtgtttgtttgcagctcgttagcatcaaatttgcaaattgcttggtagttt

SEQ ID NO: 5 - DNA, 51 bp, Homo sapiens
cttgatcaccaccatacagaaagtaccagaaaagatgttttctcttcttcc

SEQ ID NO: 6 - DNA, 51 bp, Homo sapiens
gtgtgatccaccatgcccagcctcagccttcttgaatagcaatgatcactc

SEQ ID NO: 7 - DNA, 52 bp, Homo sapiense
gtgaatttaactgcactgggtgccttatatataagtggaatcatacagtatt

SEQ ID NO: 8 - DNA, 61 bp, Homo sapiens
tcccctcccttgtttagctggcatctttagaaagtattttgccgtctagattggccttgtc

SEQ ID NO: 9 - DNA, 51352 bp, Homo sapiens
gaacgcgcgcgcctccccggctagtcccggcaggcggagccccagttagactgctcgtcaagtgtgggaaaagctccgtggcgtcacaagctactatataaaaggcggtgcccgccggggccgagtgggagtccgcggcgagcgcagcagcagggcccggtcctgcgcctcgggagtcggcgtccaggctcggagcgcgacacggagactaggtggcaggtgaggacgtccccgcgcacggccggggtggagcacgggagtggcgagcttcaggtccaccggctgccaccgaaagatgggacctgaagacaggagaggtcgctcagccaagagccccggggaggcggtggtgtctcggcggggtcagcaacggtctgtggggggtcccctgcagcgcgcagccggtttccgcctccgcagctcccagccagcgcggacgccgccctccccggcgcctcctcgctgcccttccctggccgaagccctcccctgcagcctcctccccacctgccggccctgtgttctcactgctcctctctgctctgggatccgcaggatgacgtctgtcctggttcttcttctgtctttcaaagtcactagtctccagttctaaatccgagcactcttacccattctaattctttcagtaactcagcgaaacggatggggcccttccttccccggagcactcctccagggactgagaatctgcagagccaggctcagcgctcgcctgcagagcctgcggtctgtgggacccgccagcaagatgaggaatcactttcgggaggaagggtcgtgctggggagggtcgcgccggcggggtcaagagaacctcctctagggtgggaatcagagcctccgggccgcggtgtttgagggtgagtggaattaatcccgtgatggacgtggggccttctaagggggaccaggagtttgcgaagaggtgactttgggaggggttcaggatggccagaggtggaggctgtgacctcagtgaggctggatcaagggaggggcaggtggagaagaaaccgttttaacgaggtggctttaagcggatcctaagggccccaggagatttaggaggaaggacaagaggattaagacaccgctttctggtcgggtgcggtggctcacgcctgtaatcccagcactctgggaggccgaggcgggcggatcacgaggtcaggagatggagaccatcctggctaacactgtgaaaccccgtctctactaaaaaaaagaaaaaaaagaatacaaaaaaatagccgggcgtggtggcgggcgcctgtagtcccagctactcgggaggctgaggcaggagaatggcgtgaacccgggaggcggagcttgcagtgagctgagatcgcgccactgcactccagcactccagcctgggcgacagagccagactccgtctcaaaaaaaaaaaaaaaaaaaaaaaaaaagacacggctttctgaggtgccgtgcggctgtggcgggggtggctgggggtgcgggttgtgttcattgtccaaggctctccagtgctttgcgtggcgttttggggagcacagatgaggtgtcccgacagtggggagtggctctagggagaccagaggctcgggagggggcctgggcattagaccaggcacaggatccaaagtgggaagaaatgtgggcgtagagggcaggccttggagatgggtgtgggggaggagggggagatggaaaggagtcagggaggtctcccaggctcacctggaggagtcagggtctcatttccaagtgcactgggaactgcagcctgggggtgggggttcatttggtttcaggcctgtggccaagagacctaccccctcatattcacactttggcctccacgggcttcagagatagtaccagctctttgttctcgctggttttctttcctcaccttgggctgtttcttgccttttccattctggtgttctgcaaggaccagttccttcatctcatgcacagtctacactcatggcttttgctgggagtaatgagtaaagctcccaaatagatgtttctgagttaaaacactgaacacaaaatgaagcaaaaacaacgcatttccaacctcccaaggtcacatgatgtctagaggacagtgatgggcaataactttttgtcttccccattagaatgcaagctccacgaatggaacgtggagttttttattttttattgttttttgagacggagtctggctctgtcacccaggctggagcaatcttggctcactgcaacctccgccttctgggttcaagcaattctcccgcctcagcctcctgagtagctgggtctacaggcatgcaccaccacgcccagctaatttttgtatttttagtagagacgaggtttcatcatgtttgccaggctggtccggaacccctgagctcaggcaatccgcccgacttggcctcccaaagtgctaggattacaggtgtgagccactacacacggccagaacatagagattttttgtgtttaattcacaaccgagtcttcagtgcctagaacaggtgctcagtaaatatttactgaaggaatgaattcctttttgtatttcttgtatctatcaccagatgctcttgtatctagtacctgcttggcatacagtagatagaaagctttgctgagtgaatagctctgcctttcgtcttttttgatatgtgtacatcttttaaaaaataatgaatagactttgttttagagctgttttggtttacagcaaaactaagcagaaagaaaatatccccttccacaaacatcctggactgtacagcctcccctaccatcaacatcctgcattactgtggaatatttgtcacacctgataaaccaacatagattcatggttacaaaaatgaaccaatatggataaaacatatacattatcagccaaagtccggagtttgttaggttttgctctttgtgtatattcaagatacatttgacaaatgtatagagatgtacccaaccctgtaacgtcagactccactgccctaaaaatcccttgtgcctcacctatccatccctccttccctgcaaatccctggcagccgctgatcttttttactatctccgtagtttttccttttccagaatgtcacatagttggaatcatgcattatgtagtcttctcagattggcttctttcacttagatttatacatttaggattgctccaggatttttttttttttttttttttttgtggcttcagagctcatttctttttagcacttaatattccattgtatagatgtaccacagcttatctgtccacccattaagtgatatcttggatgccctcaagttttggcaattatgaataaagctgctatagacattcttgtacaggtgtttgtatgggcataagctttcaactcgtttgggtaaataccaaggtgtggagttgcttcatcttatggtaagaagagcttggttaagaaattgccaaaatgtcttccagactctaacattaggcattcccactaggaatgtatgggagttccttgtgctccagatcctcatcaacatttgttgttgtcagtgttctgaattttagccattctaataaatgtgtaatagtatttgtttttttaaattgcaattctttaatgacatgatgttgagcatcttttcatatgcatatacatatggtgaggtgtatgttcagagcttttgcccgcctttaatcaagttgtacattttcatattgttgcattttaagagggttttttttggtatattttgggtcacagtgctttatcagatatgtcttttgcaaatattttctcccagtctgtggctcgtgttgtgaacgtggctagttctcttgacatacatatcttttcacagtatgtaatcctgttgtgctttttttttttttccttttgagacggaatcttgctcttgtcacccaggctggagtgcagtggcgcgatctcggctcactacaatctccacctcccaggttcaagggattctcctgcctcagcctcccgagtagctgggattgctggtgcccgccaccacgcccagttgatttttgtattttttagtagagatggggtttcaccatgttggccaggctggtctcgaactgctgacctcaagtgatccgcccgccttggcttcccaaagtgctgggattacaggcgtgaacaaccgcgcccggcctgatgtgcatataattttattatttgatttttaatatacatgaatagcatgcagttccaactttgctcgagacttcacaattaccattttaaagggctgcaaaataatcacatttcctcctcaacagccccactttacagatgaaggtagaggaatgctaacttgcctatgctaacccgcagagctctgaatcctccggatagcgcccccgctgtaatccacccagtgcttccctccagacccttctccctctcgtgcgcgcggaggtttcggtttcctcttttacgtaggccgccccactcgcttgaaaacaaagtccgcgatcctctagcccgcaagagagctccgggactcagaccccgggcgcgaggtcccacgagcggcggggcggcgctctcgcggtcctcctccccggcaagggccggggcagggggaggaggcagcgcgaacagtccacgccctccagccgggcgcgctcgacgcggacggcgggccagctgccggagcgcggcgactccaggggacccacagctggggcgccagagcgcggccatccgggcggggccgacggagcgcggcaggacttggctggacggcgcggccacggagaagggcgcggtgagcggactggcaccgcgcggggaacattccggaggccgccgggctcagtcccggtgacccccctggtcggggttcgcgcgccctcccccagcacgcccgggcaaggctgccgcggcgtcggagtaatccccaaccctccgccgccgcttcttccctcacccgggcctccagacgtttgccgcggaaacgaaagtaacgctcgggtgcttgcgtggaggccggggcggccgcgggcttcccggaaggagagggactctgcccggggtgcgcgcggcgggcgggccccgccggtaccggaagtcgggcggtgggcggggccgcgggcgctcccggaagtcaggggtgctccgctaggtgtctctgcttatccggagtccgctggccgcctctgtgccagaggtcggggcgctggactggtagcgggcggagacctgcctttgggccgcggttaccgcgtgagggtggaataggcagtcccacacgccattaatgttgttttggtggagaaggtgggctcctagtgccctatcattaagaaaaaacagtttgggctcttcccttaccagccctttgaaagggccttttactgttgcgcttgaggccgcagcaggttccaaaaacaaaagcatttctgcgtgatcttagacgactgaggggacgaatgtggtaaccctagcaggcttctgtcactggcaggtgcttttgtgaagtgctgcggaccctcttgagggttgggaagaggataggctgcatcctccttccctaagcatttgacaaagcctcacagggatgcgggggttgcgggggcagggggaggtctggggttgttagttctttctctcttgctgaccatttggctctcccttaatcagagctcttttttattttgggcaaagcaggaggctgttccaatgattaattcttccctgacttacacttgaagaagccaaggcaggaagtgaactttgaaatgtgcaatgtcttagtactcatctgcattctgtgactgctaggaagatggactgagtcaagaggaaattgtcttgttagaaatgtggaatactgcgtcgggcacggtggctcactcctgtaatccgagcacttcggaggccgaggtgggaggatcacctgaggtcaggagttcgagaccagcctggccaacatggcaaaaccctgtctctactaaaaatacaaaaattagccaggtgtggtggcgggtccctgtaatcccagctacgcgggaggctgaggcaggagaatggcttgaacccaggaggcggtagttgcagtgagccgagattgccacttacactctatcctgggcgacagagggagactctgtatcaaaaaaaaaaataaataaagaaatggggaatactgcagctcttctattcacaaaaagtgaagactaggcttgtttaaaatgaaatttggagtgtatcattattactgataaaatagcaaaaatgataacttttaatactcattttcctctgtctgaactatactgagcctcagagatatgttgtactttctggaatgtgaatattaatgagtattaagaaaagctcttaaacagcctctttcaaactgggcagcttactttttggtgcttattaatctataacgtgttaacatccatatgatctgggaatttgaagaaaatacttaatttcccatgctttagttggatattacattctggcaaaaaaggttgactgtattaatctgtagaacattgattttgaaccatatatttcaaaaatacatgagccgtgtatttttatttttgagacagagtcttgctctgtcacccaggctggagtgcagtggcgcgatcgcggcttaatgtaaccttcgcctcccgggttcaagcagttctcctgcctcagcctcccaagaagctgggattgtaggcatgcactaccatgcctggctaatttttgtatttttagtagagacagggcttcaccacgttggccaatctggtctcaaactcttggcaatctgccctccttggcctcccaaagtgctgggattacaggcatgagcccctgcacctcaccttcctgtatttaaaaagtttattctgtactttctccgtgtttctgactgtcgcacatttatttaatatagatgagtaaggctgtaagggtgggtctttgcatctggatttttccgaagacagattatttgctatctagagaagtagcatgtattagtgagccaatagtgaaagtttgaatgaagggacaaatatttcattccaggatcctaccactttaggtgaatcacttagctccaaatcttagtttctttatttgcaaattgaaatcatgataaaatttgcttcaggggttatgatgcttgtttaaactctgtttcaattcttgtctcttgctggtccttaagtctcttccttgctcagagcttttgtttatccaggcaaagcatcaggctgctgtaatcaaggaagttggagaaatcctctggtcacttggatagctgagcaaaatcagaattctgttagtgggaagaaagggggcaatggcaggtgggcaaccagcattttctgccattctcttgttatgtgtggtcatcatagaaaaatggggaaaaagttaattagaatattttgatatgtatacttaccctgccttagaaggtaatcgtgaaattccttgagatttcacattaatttcaatagaaacaaattcactgtcctgagtccctgaattaatatttatgcaaaacatttacaaattccttcaaatggacgtaattattagttaaaattatttgctataacaggccagcatgtctttgtcactctgtgtcatggctttgtaataatatggttatgcagtatggccatataagctgcctaaattcactgtttttgtatatccttcaactactcacaaggccttcctaagatattcagtgctcataaagttgaatctttcatgtggaaaaaagaggggtggataggaaaaacaaaagaaatcactgaacaccatgcagaagatgcagaagattaacagactaaactagcaaaccgcaagactgtaaggagaactgacttgggtcaggagctaagctcccaaagtccagtggacccaactagaggccttcccaaactccaccgcacacagcagcacctgcaggtcctagttggtcttgacaaatttgaggagatgtggctgagactgaagaagagataccaaataggactcctcataaagtagaaaccttttactcaaaagggcatttttcagggcatgttgtagttcctgcctgtttctttactgtctgtgaccttttttaaatattattaaacattctttcacaacatcgcttttgcaaactgctagatatatttcagtgttgatgggcataactgattctctcctctgtacatgctaagtgttttacctacttttgctgttctatgtaatgctaccaggaacagctttgcacataatctttatcagcatctctgatttgatccctcagagtaaagaaatactgagtcaaggaattattcctttcaaaaaaataattttaattttacatttaattcatgtctattgtggaaaatttggaaaacatagtaaaagtgtaaagaagaaagtaaaaatcaacctatcatcaagatacaatccttgttaacattttggtgtctttatgtcttttttatctgtacttatatctttatctatttacatatatatatatataaaacacacacaaatgtgaatataatttttacagatttattcgtcaaaaacaattttgaatggctatataatattctgttaggagtgtgccgtaattcatttaacaatatttttatttttatttttttggacaattaaatagttctctttttaaaatagttattcaattgatgatgggccggcatacgttgggttctgctaggctctggagatggtgcacaagacaagtttcctgccttctgtgaacttcatgagaaataattctcaattacagtactgtcttacctatggaagtgtctaatgggaagacttaggatggcttccttgacaaaatgacagttctctgacacctaaccaggcaagaagttggaagaatatggcctaagagggtggggcggatgacccttatggaatggggaaggcttcacagttctgcctgctccagtgatgtgtagtgaatgaaaaaggaggcctaaaatcaatgttatcttttatgccctctgtctccacccgctcctacctcattctcactaggattggtcagtagggccctgctgacagagtgatcccagctgagaatgttggcattctcaaagcccaaggaaacaacacccgtgtggataaacacatgtgggcggctgggccaaccatagctagagacataaagcaggcaacaaacgtgttttcacaaatatatacatatgtacatatatacgcatatacaaatatatacatatacgtatatacacatagacaaatatagctatatatctacatatacaatatatgcatacacacatacaaatatgtgtatatatacacacatatacatttttatatcatatattatatattacatatatacatatacaaatatatgtatatttatacgtatacatacatgaacgtatactttttttttctcataatccttaccacttctgtgaggtggtcattattgtttcactttgcagatgaggaaactgaggcataagtaagtgccccaaggtcacaggttggtgagtcacagagctagagttcacgctcatagtggagggcccgtgctctcggagctatggctcctggtctcttacctcacgcgtgtgcctcaccccacccctcagtcttcaggccttcatctaggcagccccccgcctccacccactgcccacgtcccaccgatgtagttgtttcctgaggcacttctcttgtttggtaatgtcttatgtcctaggaaagaaggcccataggtgctgctgtatgagcttcctccgctagacagagctggccccagcctttagatgaggtgtagaaggctggttacccgggtcacctgtagccggccactgtgcaacaccatgggcagcctaggagtttccagagtcccctctccccaaggtatttcttttttcattttttaactccctgccctccaacattttattatgaaaaacaccaagtctgaaggttacgtccaccacgactatcgtcttaacaccgcttgcttcatcgtgtaattctccatctcctttcccgtcatctgtccgtcattgtctgactttttgatgtatttccaagtacacatcagtacacatctcgaaatacttcagcatgcataccatagctagagttcaatatgtttacaggtttgtcttttgatgtaaaatttacctgctgttgttgggagtacatctgctgagtatgggcagatgcgtatgcctgcaaccctagcccccgttgatatcctacaacattatcatcaccccagaaagttccttcatgcccctgcccactcagcctcaccgtgctcacaggcggccagtgttctttttctgacacagattggttttgacttctgaaccgtcatataaatagaattacatagtatgtatgcttttgtataaggcgactttcacttatcacgtttttgcatttcgttcatgcggttttcattcatgtgtatcagtagttcatttttattgctgagtagtatcccatcgtgttcatacaccagtgtgttcactcattctcttactgatgggcagctgtttccagtttggagctattataaataaagttgctatgaacattttacaagtgttttccaacatacgttttcatttctcttgggattggaattgctgggtcatagtgcaggtgtttgtggttttataaacaaaataaatgcctatgaaatttcaatgaatattttatgccccagaaagagtatttttcatggtctgtatctggatgtagatatttattattttattttattttaatttttttgagacagagtctcactttgttgcccaagctggagtgcagtgagtggcatgcgatctcggctcactgcaacctccgcctcccaaatttaagcaattctcctgcctcagcctcctgagtagctgggattagaggcgcgtgccaccacgcctggctgatttttgtatttttagtagagacggggtttcaccatgttggccaggctggtcttgaactcctgacctcgtgatccgcccgcctcagcctcccaaagtgctgggattacagacgtgagccacggcgcccagccgggatgtgggtatttctggaatacaaaacatccatagccctccttttactctcaaagcaaaacacagacacgcacattcacatgcacacaccagtgtgtccgtgtcctgttggtttgcatggagtttctgttttgccacgaagctttttgccagcccttttcccaaagtgtttgtaccattttatactcccaccagtgatgtttgagaggtctggttgcttcacatgtttgccagtgtttggtgtattagtctttttagttttggatatcctggtagttgtgtagtggtgtctccttgtggttctccatgggtttttattctatatttcagtttggggaaaatgaatgaatggaagaaaagaagggaggaaggatgaagcaaatagctgtgctttgagatctgacactgtatacttcacagccctgatggacattctccaaattctactcctttttcacattttcttcttagagtttgcattgttctttttatattcttcagattccttcttttgttcataattacttaaacctattcattttctaatgcctttcagttgttaatttactgaagttctttggtgcttactagtgctgcttgctgtagctgcagggtctgtctcacagtgaattttttcttttgtgttttataattttggaaagggagctcatgttaaatttgtatttgtaggaattctgtctggcctggcttgaggttgttgtttgctttagcctataacctcaggatggcatcagcctgtgaccacttcctgtgtaaaagttcatagtttaggcgatcctctcaattcaaagtctgtatctatttcaagacaggcccaaggttataaatttgcacaaactttttttttttttttttaataccttgtgcccagtccaagacaaatatgtttgtttgccatgggctgctttttttctagttcatcctttcatcaaagggttgcacattgagcagtgccgtgttaaggggggattcttcattctaccttcttttcttgtattccttgacctcagctgggctttaaaccttgacttctggatggcgaggttggcactcccctgtgtgagctactgcctgcctccggtgctctgtttctgtccctagagttcttttcctttccacggagttttggtatgcatttcaatgaatgtttgtattatgttgtccaacatacttgtgtcctttatagctggagggttcttaaaattttctaatgctccatattgccagaaataaaaaggctgagggaataaatattatagtcttctctgattcttaaaaatatgaaaaatagctcatcagatgactgtaagtctatcctcagaaatccaaggcacaccaagcatgtggtggaatgttaacaaaagtttttaaaaatgatcttttctgccaggtacaatggctcacacctgtaatctcagcactttgggaggctgaggtgggaggatcccttgatcttaggagtttgagaccagcctgggcaacatagtgagatcccccggcccccccatctctacaaataattaaaaaatcagctggtcacggtggggtgcacctgtggtcccagctactcaaggaggctgagggaggaggattgcctgagcccaggaggtcgaggctccagtgagctatgattgtgccactgcactccagcctgactgacaaagtgagaccctgtctccaaaggagaaaaaaaaaaacatatatatatatatatatatatatatagtcttgccctgaggttttctctctgttttctttacagggtacagctctgccggggggggaaaaagtggtaccattttgggcgttcttgagcttcagaatgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagtgagtagggagcaggggtgggtgggctaagttctgagcgggatggggacgttggctgttgtggagccgttggctgctgcggagccgttggtgggtagagtgagagggagcctgccaagatgtcagggtgacgatggctcttcggattgttattgtcgctgatgtggactctgagactgggtagtgaaggactctcacagctggtaagtaacagaagagacatgaacaagaaggaaatagagaaagacaaagagataagtgtggtggggggacaatgaaaaggaaacaggaggatgggtgaagacagacctattcactcgcttcacattttcagtgaggaccagccctaagtataaaaatgaaaaggcagttcattcctgagaggttctgagagccttccctttaaagcaaccagtagagtggtttcacctctctccctcctttgtcagagtggttttgagagaggctgttttttttttttttaatcgttctgtccagcacggtcttttttcaggttaccttgctgctgttcatgtacttttcattcattgattcattcattccttcattgatttacatttttggagcatgcactgccagttactgggcaggagtactgtgaggaagtgagaaaccgctctgccttctaacgcttctggcctgtgcctggaagagatcggtacagacccaagcatgaccccagcacgtgggagtgatgtgttggaggtgtgcaccaggctcttgaggcgctggctctgccagcctcagcatagcacctttcctgcccacaggcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgaggtagagagggcccctccgcacggggatccccagcgcttgggccctggctgtctgttatggctccccttggggtgtttgtttgcagctcgttagcatcaaatttgcaaattgcttggtagtttgaattttcttttcctttttttatagagacaaggtcttgctatgttgcccaggctgaactccaacacctggtctcaagtgatcctcccacctcagcctctcaagtagctgggattacaggctggatttcctgaagtcagtttctccttaaaacatctatgccttgattgggctccgtgggtcacttctgtaatctcagcacttttggcaggaggatcacttgagccttggtgctcaagaccagcctggataacacagtgaaatcccctctctacaaaaaataaaaggtgcatgtgaacagagcaagaccctgtctctgaaaaaaagagaaaatcttccttgccttttactgccctgagagtggtctcttcatcctctcgatcctgtggacagtcctccatagtctgtgtcttaggaatgtgattggctgtttaccatacctgtccgctgctctgcaatctacagttcagagacaagccctgtcttcatgatgcagacatcatctgatgtagagtatatgcatgatgggatgacattaaatatggattaatgaaaggatatacactataaatgaccgtgagagcagatagaatattagtggttgcactttgataccattcaacaaagttttattttaaattaaatatagaaattattggcaacaacactggataggatttaaaacaaaaataaaaattgtttaccaaagtcaaatgatttgaaaacatttttaaaagcttatgtgcctgttaagatgaaggccttgcgctagttgctcatgaatcaatagctaatatgacgtaggagagtaaaaggaggcagatagctaaataagtggtatggtgtgatggggttttttttttttcttttgtaagagaacaaggtctcactgtgttgcccaggctggagtgcagtggcacaatcatagtgcactgcaggcttgaactcctggccttaaatgatcctctcacctcagcctcctgagtagctgggactatgggcatgagccactgcacccagctgtgtggtggtatttttttttttagaggagtctcgctctgtcgcccaggctggagtgcaatggtgcaatctcggctcactgcaacctccacctccctggttcaagcaactctcctgcctcagcctcttgagtagctgggattacaggcgcctaccaccatgcctggctatttttttaatttttagtagagacaagttttcaccatgttggtcaggctggtctcgaactcctgacctcgtgatccgactgccttggcctcccgaagttctgggattacaggcatgagccaccgttcccagctgatggtgttttgatagacatactcattgctgagggagtacacatgagaggcacccactcagcctggaatagacagaaaaggcttccaatgtgaaggacatgtagacgttttgaagcagagaattagagaacaagtgttctaggcagagagaataccatgtagagactgccaaatttggggaactgtaaattcctgggtttggctagagcatagattgcacagggtatggggaggggtggcgggatatggctgcctttatggcaaatactcccacttttccactttcttctcccaccccacaattgccaagccaacagacatctcacaaaaatacccttgggaaggagaagaggtgatcacttatacgcatggtattatagtttgatcagattgctctccaccccagaactagagaagatcagctgtttgaagtaaacattttgattgggaaataagtctttggtctcccatccaagtactaaccaggcccaaccctgcttagcttctgagatgagagagaaatcattggggacatgaggcattgtgcaagatgagggaaagttcaggtgcacctgtttggactcagtcagggcatcaggaggacctgagagagaagcagaaagagaggagagagggggaccaactttgcttactcagaagtttttcctaagtccaaaggtataccaaggaagaagagaaaacatcttttctgctactttctgtatggtggtgatcaagctattaacagtgtagctctgctacgttatgtcataaatatggtttgagggggaatctcacttctatcattaaaatttttttcctgttaaccaacgaccaaaatacatatacatttttaggaaacatcttgtttgtatactgattttttattgttaagcaaaactgtaatttaaccactataatagaaactggaattgtttctgttagtattgatgctcacataacaagaatacaattataagaaccagtgacctgagactctacagaagcctgaagtatctgctaccaggtcatcaaatagatattaattcacttaagcgtagttttaacatcatgtttatctcagatttttctatgattgtaatcagtgtgaacatgttcccttgtattcttttttctcttaatgttattttacacgcgacatttgcatggcattgttcatgtccttaatgtaaacttagtatgatgtcgtgtgagtatcccacagaaactgcttgctcgtatatttattcatccattcattcaacaagcattccaggtacaatcctaggttctaaagatacagtcctaaataatatatatttttgttctcaaggagcttaattttatagtattttatgcattaccaaattattgtgggatttttgtttttacactaacaaaatacaacacgaataccctctgcccccccccgaatgaaatctttcttgtttcccagtgacagcagtactgtggtgcacctgctggaaggaaacctggtaaccttctcctcaggctcagatcccaccaccctttcatgaaacagttgctttggcctatttgataacctttccatttcatagacttaatatcacttgttttagatgagtactccgctttctgtaaaaggaaggataattttggaaatcattgaaggaatgtgctacttacatggaaaaggcgtgatacacaaggacctgaagcctgaaaatatccttgttgataatgacttccacattaaggtaaaccatcatcggtaggcttccagaaagttgggtgattttagttttaattctccaatgtaaatccattctcgtacattggagaagctaaaggaagacctagctcagcttataactgttgatgatggtgccgaaaggctctaccggtgattgagaggaaggctgcaggcttgatgggatcaggagataactgtcaggataactcatatcttttgggggaggagtggagttgaatggggtagatgagaagacctctttcctagactgtgttttttcctctgttagaaacatttagaaacattcaagggttgcccttttcatcctggaatgggccattggtgtcataatgttctccaggaaatctgtgtcaccaagactctcctcctctggaaatgcaggggaatgtgaaggtgtgtgcggaaaatccttagagttcagatatggcctaagagtgagatcagcggccgggtgcagtggcccacgcctgttatctcagcactttgggaggctgaggtgggcggatcacctgaggtcgggagttcgagaccagcctgaccaacatggagaaaccccatctctactgaaaatacaaaatcagccgggcgtggtggtgcatgcctgtaattccagctactcgggaggctggggcaggagaatcgcctgaaccggggaggtggaggttgtggtgagccgagattgtgccatcgcactccagcctgggcaacaagagcgaaactctgccttaaaaaaaaaaaaaaaaaaaaaaaaaaaaaaaaaaaaaaaaaaaaatcagcagtgcatcaaaaacctctactttgaaattttgttttatatttgtattcgattgtttacttctgtctgtattttaaaaatcagtttcctttgaacacattctttccattgtcgtaaagggaacaagggaaagggatttttcttttgttcattcaagggcaggatttagctagggggagtgtcatatctaattggcttctgcagcaggaatccaagcacaagaatccaaaaggggaagcggaaggggaagcagccctgggggaggcacccaggagctgctgacacaggggcctctcatcaccagcaggttctgcgtctagcgctttgtcctagtcttggacaaagggctgggaataaaaattccttattcataacctatcttcaaattcatgacttccctcttatgtcgtgggttagaatctgtgaattttaggtactcatgtgccacagttcatccccacatgcttgcttggattcctggaggcagaagtggaactggcacctatattctcttctgaactgtgctgccctgtcattttgccctgccagcactgttcttgttctgtgtctgaccttgctatttttctctctgcttttttgtctgtttttctcctcctttccaccctatctcctcttaggttcaggctacagctcaaaacaagcaacactgcagtccttatcctcagtgtgtctttgggatccttggacacttgcatccatgggcctctggacgccatttggggaaatagaaggtgtagtatttagccactgtctgctgcataaatgtgcagagaagaaaataaccattttccccccaagccctagaaatagttgaaaaacatacgcagagtcaatggggaatgagaataaatatgtttggttgttactgttcttgtcagaaacatcttccgcttttcccctcccccttactgcataaggaatacgataccagaatttcatgtgaacgtttcctgggcgacattttacaaatggctgaggagacacacagtttctatgagtctgttgagaagtcgctgttttgaacctgcccagtgcaaccatttctggaaaatttaccgtaccttatgtataatggataagcccaaaattgaaagtcagatctgatttgcttacggccttcaccaaacaatcccagtggctcaatgtctttcgcagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaagtaaggcattacttactttccactgccgtcccctcagcatctacacgcactgtgcctggaactaataggtgcccagtaaatttttgttgaatgaatgagtaatcccatttaggaatcagtgatcataggatgaataggaacctcgatagtgtgtcattagctggagaggtgctgtgaaattccagggttcttcctagacaggaaagttgattgtttctgcttgtgaggacttagcctttcacactacaaggcatacacttcatactttcactcattcacttgctccttcattcaagaattatttcttttcagtccctactgtgggcacttgagtcggcattgggaatataaagatgagtaagtcgtggcttgtgtaggatagagacactctgcagtttgttaagtgcataaactgatggatgttgtcctacatactcttcttgggtgatcatcagctcccatggtttgatgatcatctctttgcaaatgcttcttaaatctgtgtctctggctctggaatggatatccacttgtcttacagtcagatccatcgcaggtaccagaggaacctccaaagcagcatcccccaaacacatcctgtcgacactgtcctgcagctcgccccgcttcacccctcttcatatgcttgggaagtggcatcgccatccttttaccaagttgttgcccaataagaaacttcagagtcatcccagactcgcccttgcccctctctcccacattctatcagatctcaaaacctgtgaattcaagatatatgtatgactcaagatatatctgcaacccatctgcctttctctgtctccactggcctctgctcagttcaaaccctcctcgtttctgcagcaggctccttccaccgtccaccattcatgtggcagccaaaatggtcgtcctaaaaggcacatgcaaccctctccctcacatattggaaatccttcccagatgctctgtggtcttcaggatgaagttcgaattcctcagcgtggaataaaaggccttcatgttctggcctttgcttctctactggcctcgtccctaaccctcccttctttgtcagtgtgcagttacactgaggaatttgtgggatgcgtcatgatctctgtaacttctagtctgttgatatcctgcttctcttcctggagtcctcctccttaagtctccctttcccattctttacctgctaatttttctggactcaacaaagccttcctggacctttttagtcaggtttgtgctactggaaccccttgtacatccttttttttttttttttcccgtgagacagagtctcattctgtcgccaggttggagtgcagtggtgcagtctcggctcactgcagcctccacctcccaggttcaagcaattcttctgcctcagcctcccgagtagctgggactacaggtgcacaccaccatgcccagctaatttttatatttttagtagagacggggtttcaccatgttggccaggatggtcttgatctcttgacgtcatgatccgcctgtctcaacctcccaaagtgctgggattacaggtgtgatccaccatgcccagcctcagccttcttgaatagcaatgatcactctgtgttttaagtcactctcattaaactgagctcctaaagacaaggactgccctgtatttgcttttgcacatggagcatgccattggccttgcccacataatctttttgttccatcagcaggcagctttgtcgagaattttgttttatctgcagacattaagatattgtttggtcatctctgtagtggaatatgtcagctccttgtcataaggccattccactctatggaatgaggcagctgtaccagaggctgagaaaaatgagcagtattgttcaagttctgtcaccttcctgcctgagagaggagcaagacctgaaagaaagtctttgctttgtagatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggtaagagcatcttttctgactgtgtaggatgcatcctgtgtggtgattttcctagtaacatattgttaggaacttggtttgaatcctcagaaaactgagttcgacttgacttgtggttttaaactgattttctagatttcaaacatgttattcagtgcaaagaagccctctctctcaaaagcatgatggcacataaggccttcttgtttcctttttttgtaattaactttttaacttgggtttaaatatacgtaacatgaaatttaacattgtaactgttttcaagtgcactgttcaggggcagtaagtacagtcccatagtacattcactttccatcgtctctctccctgcccctggcagcccctatcctactttctgtctctgtgaatttaactgcactgggtgccttatatataagtggaatcatacagtatttgtccttttgtgtctggcttactccacttggcatgatgtcctcgaggttcatctgtgttgtagcatatgtcagaatttctttccttttcaaggctgaataatattccattgtatgaatggagtgcattttgtttatccattcgtccgtctatgggcacttaggtggctttgaccagccttcttgcttcttcacttcttttatttccatcccctcccttgtttagctggcatctttagaaagtattttgccgtctagattggccttgtcatacagggatgaggaaaggtgccgggatgtttgctgagcagtattctatggtgtggatgtaccacggtttggttaactactcactccttgaaagacttgtttccagttcttaactgttgggactaatgcagctgtaaatattcgtggacatgtttttgtgtgaatataagtcttcatttctctggagtaatacccaggggtacaatagctaggttgtatggtagtttcatgtacagtttattttaaaaattccctaacattttcccagaggggctgtgccattttatgttcggtgtccgagtgatgcaggttcttgtatcctcaccagcatggggtacccaggctcctcacacttctgcttagtcagctacaaatttatttgagtattcccacaagccccacttcaggtttggtaattcgttagaaagactcacataactcaagaaaacattttacttacgtctccccttttactgcagtgggtacaattcagaaacagccaaatggaagagatgtacaaggtgaggtatgggaggagggtgtatagaacttccatagcctttcaagacacaccgcattccagtgcttctgtgtgttcagcaacctcaagctctccaagcctcaccgtttaaggtgatttatggaagttccattacatacgcatgactgactaaatcatggtcattggtgcttgagcttaatctccagtccctctctcctcactggaagtcaaaggtggagctgaaagtcccaaccttctaattatagcttggtctttctggttgctagcccccattctgaagttgcctagttttctgaccccacccaaaagacactcttagaagactccaaggattttaggtgctacctgccatgaacaaaatcaaaccttcctgattctccaaggtgtcttcttttattgtttcctttctgtttagagaactttccttaatcattcttttaggatatgtctgaactctgtcattctaaaatttttttccttatagggaaggtgttatttttctcttgctgctttcaagactttgtctttatttttcagagatttagttgttatttatcttggtgtggattcctttgggtttattctgtttaggttttgctcagcttcttaaatctgtaggtttatgcctcttaaaccatatttggggagctttcagccattatttctgtaagtactttttaacccccaccctctttctcttttctttctggaattctgacaacataaaaactatatctttggttatggttatagtcccacaggtccctgaggctttgtgttttttttgttttttttgtttttgtttgtttttttcagttaattttctctctcttattcagactgggtaagttctatcattctgtcttccagttcactgatttttctgtttctcctcgattccacagttgaacctgtctactgagcttttttttttttttgacacggactctcactctgtcgcccaggttggagtgcagtggcgcaagctctgcctcctgggttcacgccattttcctgcctcagcctcccgagtagctgggactacagacacccgccaccacacctggctaattttttgtatttttagtagagacggggtttcaccgtgttagccaggatggtctccatctcctgaccttgtgatcagcccgcctcggcctcccaaagtgtgggattacaggtgtgagccactgtgtccggcctaccgagctttttattatgttttggtcattaaagttttcagctctaaaatttatattctccccttttttgctgagactttctatttttcatttgtttcaagcaggttcataactattaattgaatcgtttttatcatggctgttttaaaatccttgacaggtaattctgacattgctgtcatctcagtattggcatctattgattgtcttatttcattcagtttctgattcttaatgtaatgggtaatttttctgttgaaacttgtacattttcttcctatgtttggagattctgtatcctctttaagccttctgttttaactgaactttcctgacactgctctgacaagggaagagtggtggccaccttattagtgctgggtagaggtagaagttcagcctctcgactcagcccttattgacacctaaggtcagaggctcctcttcactgctgatcaggggtgggaattctggcagagtccacacggtccccactgacacagaggtgaaggtacctcattactactggacaatgttaaaagtcccgattctccactagacctcctctgacacttccccagtggggaagggaagggtggtcttgttactgccatgtggggcaaatccaggctattcttgtgttctcctgacaccatgggagtggaggcgctcattatcagctggcaggtaggacagccctggcttcctgttgggccttctctgacattaccccggtgggggtgttgggttacgttgttactgtctcatgcggttggatgttgaggctctccacttggccttagctgctgtgggtggggttggagccacagggttttttctgtggtggttggctggaacagagtggttgctgactaaaagttttctcctatccaagtactaaccagacctgaccctgcctaacttccaagatcagacaagtttgggcatattcaggatggcatggctgtagactggctaaaggttttctgtctgtcccttgctgccccttgcttagttctttggctagaaagagcaggctttagttggagatttttttttatctgtggctcttgatggtttgaggttgccaccttttcagccaagtctgagatatatgaggaaaagagaaaatccagtgaactcactatcgtgtgtttcatctgatcccgaggtttcctgctggtctgcctccttctcttctcctttcagagactctgtatgtttgttttataaataatatataggatgtttttgttgtacttagaaggaagattaagaaaaaagtatgtccactccatcttcctggaactggaagtttcttttttcttttaaggctaacagctgtaacccattacactaaatccttcctctgactatcccaatctctcataaatttgatgccagaggaacagaaacatggagttcgcccatccctgttcagctggagatgaactcacatcagggagcagtttgtgttgcagccagagaaactgcagcacgaataaaaagcacaaagagcacaggctattgaggcagaaacccctggtttcctgtgttagcctatcccttgttctgtctattaatcttattagtctctatcagagtcagtgatttggagcttaaatttttatttaagctttgcccacagattgaggtaagtaattcaaagataatagatggctaatatttttattttatgttagaaaataattaagaaatttaaagtacattttacttttacaggcattgaagaaaaatttaggcctttttatttaagtcaattagaagaaagtgtagaagaggacgtgaagagtttaaaggtaggcaatatagcagatgctcaaaatattaacatagcaaaatatatactgtcaatcatgaaaaccaaaacaaagtgatttgttattgagatttgaggtaagctagcttggtaaacatataaaacaaaatgagcaattgtctgcacatatcatcctgttggcatactggacccatttctccttcaaaactcatgaaatgagagaaaacatatatagcaatatgaaaacaaaagcacaatgtcactggcaaagattggaaagtaccataagtggatcagccattttgaggaattcttgaaagatagaaagaccacagaattattctgctgagacatataacagacaaggaatactagcccaagacataatgctgaggaagaagtaccggaggggcagtcttgggtcagtgtgcagaagcaacagcacagtcacctgggatgttgacaggggagtcatctgagaacctcatttatgctatttaggaaagtcggcttctctccctccttcccctgtacttagcaacagatggcagcagcatctgcccaagactgaagcaaggaggtggtttctggggttagaggggcatgacaaggcccagttaggaaccaatcaactaagaggaatggagaccaccccgcccccctgccaactctggaatcaagctactgccatccatcttccagcaacctaccccataatcatgaacaggagactacgacaaataaaacaggcagagactttaaaatataatttaaaaaagagattagaatcagctaagataaactctaaggcaaaaaatcattccctcaaacaatcaggaatatttcataatgataaaaggcccaattcatctggaaagtataaccgctttaaatgtggatccaccttcaataacatgacctcaaaatcaaccagccaaaagtgacagaactcaaagaaaaacagacaaatccacaattaaaactgaagattttaatacacatctctcttcaattgatagaacgagcaaacaaaaatcagtaaggatatagaagacagaaagaacatgacgaacacacttgacgtaacggaaacacaggatacgctgcacctaataaccgcagcgcacctacctgccgcacctagtaaccgcagagcgcctacctgccgcacctagtaaccgcagcgcacctacctgccgcacctagtaaccgcagagtacctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagagcgcctacctgccgcacctagtaaccgcagagcgcctacctgccgcacctagtaaccgcagagcgcctacctgccgcacctagtaaccgcagagcgcctacctgccgcacctagtaaccgcagcgcgcctacctgccgcacctagtaaccgcagagtacctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagagcgcctacctgccgcacctagtaaccgcagagcgcctacctgccgcacctagtaaccgcagagcgcctacctgccgcacctagtaaccgcagagcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagagcgcctacctgccgcacctagtaaccgcagcgcgcctacctgccgcacctagtaaccgcagcgcgcctacctgccgcacctagtaaccgcagagcgcctacctgccgcacctagtaaccgcagagcgcctacctgccgcacctagtaaccgcagagcgcctacctgccgcacctagtaaccgcagagcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagagcgcctacctgccgcacctcggaaccgcagagcgcctacctgccgcacctcggaaccgcagagcgcctacctgccgcacctcggaaccgcagagcgcctacctgccgcacctcggaaccgcagagcgcctacctgccgcacctcggaaccgcagagcgcctacctgccgcacctcggaaccgcagagcgcctacctgccgcacctcggaaccgcagagcgcctacctgccgcacctcggaaccgcagcgcgcctacctgccgcacctcggaaccgcagcgcgcctacctgccgcacctcggaaccgcagcgcgcctacctgccgcacctcggaaccgcagcgcgcctacctgccgcacctcggaaccgcagcgcgcctacctgccgcacctagtaaccgcagcgcgcctacctgccgcacctagtaaccgcagagcgcctacctgccgcacctagtaaccgcagagcgcctacctgccgcacctagtaaccgcagagcgcctacctgccgcacctagtaaccgcagagcgcctacctgccgcacctagtaaccgcagagcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagagcgcctacctgccgcacctcggaaccgcagagcgcctacctgccgcacctaggaaccgcagagcgcctacctgccgcacctaggaaccgcagagcgcctacctgccgcacctagtaaccgcagagcgcctacctgccgcacctaggaaccgcagagcgcctacctgccgcacctagtaaccgcagagcgcctacctgccgcacctaggaaccgcagagcgcctacctgccgcacctagtaaccgcagagcgcctacctgccgcacctagtaaccgcagagcgcctacctgccgcacctagtaaccgcagagcgcctacctgccgcacctagtaaccgcagagcgcctacctgccgcacctagtaaccgcagagcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaaccgcagcgcgcctacctgccgcacctagtaactgcagagcgcctacctgccgcacctagtaactgcagcgcgcctacctgccgcacctagtaactgcagcgcgcctacctgccgcacctagtaactgcagcgcgcctacctcctgcacctagtaactgcagagtacctacctgccgcacctagtaactgcagagtacctacctgccgcacctagtaactgtagcgtacctacctgctgcacctagtaactgcagagtacctacctgctgcacctagtaactgcagtgcacctacctttaaaggcttttcaagaacatacagagcattttcaaaaactgaccatgtgctgggaaatcaaataagtctcaatttaaaaggattgtaatcataaaaatgtcctctgagtatatttcaattatgttcaaaattaattttaaaaaggtaattggaaaatcttatgtggttagaagtaacgaaatatacttttaaatatcccttggatcaaaaaaaataaatcacaatggtatttagaaaatatttttcactgaaggctaacaaaacactacatattaaaattataaaatgcactttgcaaaggagtacttaatataaccttaaataaataaatatttcatatatatatatatatatatgaagaaaggctgaaaattaattagccaaacattatctgaagaagatagcaaataaacctagagaaaaaagaagtaaataataaggaaaagagcataaattaatgccgcagaaagtaactaacagcggatcaaaaaatccaaatattggttattcgaaaggctaatacaatagactaattaagactgatagagccaggtgtgaggtaatcccagttacttgggtggctggggtgagaggatcgtttgaggccaggagtttgagaccagcctaggccacacagtgagaccccatctctaaaaaaataaaataaaaaattaatgagacatggtggcatgtgccagtagtcccatgaaggctgagatgggaggactgcttgagcccaggagtttgaggctggagtaagctatgatcatgccgctgcactccaacctgggcaacagagtgagaccctacttctgagaaaaaaaagaaaaaaaagactgacaaaaaaggagacaagattgaaatccatgaataaggaggaatcactataggtcctgcagacatcaaatgttattaagagaatattatgaacaactttttgttaattattttaaaatttaggtaaaatggaccaatgtctttaaaaagttcaatttagcaaagctaactcaaatagagatagaaacttagaatattcctgtaactattaaaagaagcagtgataaaaaaagaaaacttcctatagagaaaaccccacagagagcttcatcaatgatttctgccaaatattcaatgaagaaattatgtcaatcttatacagttttctctagataatagagaagagagaactcttccagattcactttgtgtaagcataaccttgattcccaaaactggaaaataaggtacaagaaaggaaaattacagaccaatcttgtttaacagtatagatgcaaaagttctaaataaaatactataaacaaactcaattcatcaaagaacaaacgcaaaattcggtttctgccaagaatgcaaagttagtttaactttatgaaaatctgtcattataatttattacattgtcttcttaaatgagaaaaagatatatcttttcaatagatgcagataaatctgttgataaaatgtaatgtttattcatgattaaaaatgctagcaagtcagaaaagaaattccttttctgataataaggtatcttgaaacgaacagaagttttgtacttattttaacaaaatgttaacagccttttttttttttttttgaggcaaggtcttactctgttgcctaggctggagtgcagtggcgtgatcacggcttactgtatccttgacctcccaggccgaagcaatcctctcacctcagccccctgagggaccacaggcatgcaccaccatgcctggctaatttgtattttttgtagagacaggattttgccatgttgcccaggctggtctcaaactcatgagctcaagccatctgccctcttcagcttcccaaagtgctggaattacaaggcatgagccactgtgtccagcccacctttctctgaaataatatgcaagaccaagctacttaccacccctatttctggtcagcctggtactggaggccctagctattttagtagagcaagaaaaagaaagaagagatataggattggaaaagaagaaacaaaactgttcataggttataggatgtgtacatagaaaaactcaaaagtatcttttaaattaataatagtttagttataaaaatcaatatataaaaataaatcttacaaaaggtatgtagtaactctaaaagtataaccttaattgagagacataagaccaaagtaagtgaagaaatataccatttactttgtgattcggaagtcaccatagtacgaagatgtcagctcttctcaaattgatctatagaatcaatgcaatctcaaccaatatctcaagtttttttttttttttttttttttttttgagacagagtctcgctctgtctcccaggctggagtgcagtggcatgatctcggctcactgcaagctctgcctcctgggttcatacaattctcctgcctcagtctcctgagtagctgggactacaggcgcccaccaccacacccagcgaattttttttttttttaatagagacggtgtttcaccgtgtcagctaagatggtctcgatctcccgacctcgtgatctgcccgtctcagcctcccagagtgctgggattacaggtgtgaggcaccgtgcccagccaagtattttgttttgttttgttttgtttttgagacagagtcttgctctgttgcccacgctggagttcagaggcgcaatctcggctcactgaaacctctgcctcccgcgttcaagcaattctcctacctcagcctcccgagtagctgggattaaaagcgtgcgccaccatgcccggctaatttttgtatttttagtagacatggggtttcactatgttggccaggctggtctcaaactcctgacctcaaatggtccgcctgccttggcctcccaaagggctgggattacaggtgtgagccaccacgcccagcctatatctcaaccttttagttttgtgtgtgtgtggcaagtgggtgtgaggagtggaacttgacaaacctactctgaaatccatatgaaaaggtaaagaagagaataagatcagccaggatgcccttgaagaaggacaagactagtagtgaagggtttgccttatcagattcttgtcaagaattattataaagctatattaaaaagacagtggtattggtgtaggttggacagatactgttggaatggagttgaaaacccatagatctacacatacctagaaacttgattttcacagagatggcactgtagagcagtggggaagtgacagtcttttcagtaaatggtgctgagacaactgggtatccacatggtacagaaatgaaattgggcttctaccttacaccataccaaaaaatcagctccaggttgattactgacagaaatgtgaaagataaaattataaagctattggaaaatataggagaatttgtccatatcctcagggtaggggatcctcaacagtatacaaaagcattagctgtagtggtaagaactgataaattttaaaatttataaaattttaaatgaatgtttaaaacgaatttataaacgaattcattgtaaaattctataaattatttataaataaatttatacatttataaatttagaagatttttaaatgaatgtatacattcattttaaaacttttattcatcaaaataaaccattaagagaatggaggcagggcatggtggctcatgcctgtaatcccagcactttgggaggctgaggtgggaggatcgcttgaggccaggagtttgagactagcctgggcaacatagtgagacctcctctttacaaataataattaagaaattagctgggcgtggtggtgcccactttcggtcccagctactcaggaggctgagatgagatgactgcttgggcccaggaggctgcgccattgcgctccagcctgggttactgaatgagaccccatctccaataaataaataaataaataaaccagtcacagaatgggagaagtgcaacacacacataactgacaaaggacttgaatacataaaatctagagaaattgtataaatcggaaaggaaaagatatgcaacccagttaaaacagggatgaaatagctgaatgggcacttcaccaaagagaataaatacaccaaaaaagtgcacaactttactggtaattagagaaatgcaaattaaaaccacaataagataatatgtgtctaaacattggcaaatatggcaaggcttttctaaacaacatgaaataaatccaaataaacagtgaaatgtgtttatggatggaaagacttgacatactaaagctgtctattctccccaacatgatttataaaaggagtgcaatcctaattaaaagcacaaccaaattctttatggaattcaacaagctgactcttaagttacacaaaatgtacaaagatagctaagaaaattctgaaggaggagatcagaacagtgtagactacagcaaggataatcaaatagattaatagaaaaggtgagtctttgcaggttgggttttccagaagcagccactgagttggagattggggtgcaaggaaggagtgggaagcaggaatgagcagaggaagaagtcagaaggcaacgaagcctgacagctctggcagccgggagcactgagcaaatgcccctcaggcttgtcccacgtcagactgaaatggccaaggctttatcccctcgcctttctcagtcactggctcagaaggggagtacctggggcaaaggagttcacagctgcaggctgtttgttcactcagctccttgcaacagggcagcaaatccctccttgagggggaatctgagaggcccatcactgtgtctaccacaaagtccaaaatcaatccatgtatgtttcttaagccacacacaaaattcgcaaggtataaagaaaaagattattgtatatgacttcatgaaaatggaaaggtagtcaaaaaggagaaattgtgtttagcaaaggattaatatccaggatatttaatagagtgcctacaaataaaaaacaaaaataggccgggtgcggtggctgacgcctgtaatcccagcactttgggaggccgaggctggtggatcacgagatcaggagatcgagaccatcctggctaacatggtgaaaccccatctctactaaaaatacaaaaaattagccaggcgtggtggcaggcacctgcagtcccagctacttgggaggctgaggcaggagaatggtgtgaacccaggaggcggagcttgcagtgagccgagatggagccactgcactccagcctgggtgacaaagtgagactccgtctcaaaaaaacaaacaaaaaaaccaaaataaaagaaaaataatagacaaagaacatagacaattcaaaaaagaaactgaacaacatacttcagaaaagatatttggcctcgccaacagagaaaggcaaaatgaacccgttacaatttttcatccattagattgaaatttttgaaagattagtgatacagtgtgttggtgaaaatgcagaataacaagcagtcttctgcagtgttggtaagagtggcgaagtttattagaaggtgaggcacccaacaaactccacaagcccataagtttggctgcagttctactctcagtaatctatactgctcacgtgcactcaaagatgtagtttccattctttgcaccttagtttataagagtgaaaaattagaaacaactcaaatgtccataagtaactgccctaaattatgattcatctaaactacagaatactaatagccatttaaaagaaagaggtggaaactaacatgtaatgatatagaaagatcttcaagattagctgttagtgaaaaaaataagtcacaaaacaatacttacagtgtggtccttttaatgttaaagatatatatattttggcctggtgcagtggctcatgcctgtaattccagcactatgggaggcggagttgtacagatcacttgaggtgaggagttggagaccagctggccaacatggtaaaaccccgtctctactaataatacaaaaaaaactggccaggtatggtggtgcatgtccataatcccagctactcaggaggctgaggcatgagaattgcttgaacccgggaggtggaggttgcagtgagccaagattgtgccactacactccatcctgggcaacagagtgaaactgtgtctcaaatatatatatatttctggaatgtgtgtgcaaattcctagagagagaataagagtgatatgcatcacactctggataaggcttacctctagagaatggaagagtaggtgctgaaggaatgtttggatgttattctatgtgattctgcattgtttgaatctttgggggtttttttggttttctttttgagacggtcttactctgtcacccaggctggagtgcagtgacacaatctgggctcactacaacctccacctcccgggttcaagtgattctcgtgcctcagcctcccgagtagctgggattacaggcgtgtaccaccacacccagctaatttttttgtacttttagtagagatggggtttcaccatgttggccaggctggtcttgaactcctgacctcaagcgatctgctcgcctcggcctcctagagtgctgggattacaggcgtgagccaccatgtctggcctgtttgaatctttcaaaaggactttgtacttaacatatttgtgtaataaaaataactataaaaagaaaatcaatttgataaaattaaaataacaaaaatacttttcatattaggctaagcagggtggctcgtgcctgtaatctcagcactttgggaggctgaggtgggtggatttcttgaggccagttcaaaaccagcaacatggcaaaaaccgatctctacaaaaaacacaaaaattagccaagcgtggtggcgcatgcctgttgtcccagctacacaggaggctgaggtggcaggctctcttgagcctggaggttgagcctgcactctagcctggaggacacagtgagactctgtctcaagaaaaaaaacaacaaaaaacccttttcatgttaattaaaaacaataaacatacagctgggtgaggtggctcatgcctgtaatcccagctctttgggaggacaagctgggaggattgcttgagcccaggagttggaggtttcagtgagctatgaccatgccactgcactcctgcctgggcgacagaggcagatttgatctctaaaaataaacatgcaaagagacaaaaacaatatatagaatgctacaaagtgtgaacataattaataccctctgtcattctcccctagatcatgcatgtattcatttagctagtatttttgaccacttatttatatagcagacaatcgttaacagcaaaatacaaataaaactaggaaaaaaaaaacttgaaggaaaaataccaaagtgttaacattttatctttgggtgtagggcaaaactatggataggttttaagaatatataattttctatgttttctataatgaggattattgctcctataaggaaaaagttttttactgtggcaaaatatacttaacataaaattctctattttaaccatttttaagtgaacaatccagtggcattaagtacattcataatgttgtgcggccattaccactatctatatctagaacttttttatcatcccaaactgaaactctgtacctgctaaacggtaactccccattcctccctctcccagcccctggtagcttctattctgttttctgtctctgaattctaggtagctcctagcaaaaggatcacacaatgtatttcctttggagtctggcatatttcacttagcataatgttttcaaggttcatctgtttcatagcatgtatcagaatttcattcccttttatggctgaataacattccatcatatggatatacaggttgaacatccctaaacccaaaatccaaaattcaaaatcctccaaaatctgaaacgttttgagtgccagcatgatgccacaagtggaaaataccacacctgacacctctgctttctgatagttcagtatcctcaaactttgtttcatgcacaaaattattaaaatatggtataaagtgaccttcaggctatgtgtataaggtgcatatgaaacacaaataaacttcatgtttagacttaggtcctctccccacgatctgtcattctgtacatgcaaatattcccaaattggaaacaatccaaaatccaaaacacttctaatcccaagcattttggataagggatactacacctgcaccatattttgagttcctgccttcagttctttggggtatatatatccgggtgtggaattgctagatcatatggtaattctatgtataactttttgtttttctgagacagagtctccctctgttgcccaggctggaatgcagtggcaccatctcggctcactgcaacctccgcctcccgggttcaagcgattctcctgcctcaccttcccgagtagctgggattacaggcatacaccaccacgcccggctagttttttgtatttttagtagagatggggtggtttcaccatgttgtccaggctggtctcaaactcctaacctcaggtgatctgaggtcccaaagtgctgggattacaggcgtgagctcccatgcccggccatgtttaacttttaagtactgttaaaattgttttacatggtagctacaccattttatattccccaccagcaatgcacaaggattctaatttctccaccatcttaccaacactttttatttaccatttttatattatagtcactctaaggagtgtgaaagtgtacctcatggtggttttgacttgcatttccctatgactcatgatgttgagcatcttcatggcttattggccatttgtatatcttctatggagaaactgctattcaagttctttgcccatttttgaattgggttgtttgttttttgttgttgagttgtaggattttttttttttatgttctggatttcatccgttttcagatatatattgtttgcaactttttccccattattttattttatttatttattttttttttggagacagaatcttgctctgtcgccacccagactggagtgcagtggcagaatgttggttcactgcaacctctgcctcccaggttcaagagataactcctgcctcagcctcccgagtagctaggactacaggcacacaccatcatgcctggttagtttttgtatttttagtagagacaaggtttcaccatgttggcctggcaggtctcgaactcctgacctcatgggatccacctgccttggcctcccaaagtattttttccttattctgtaggttatctttttactcttttcatagtatcctttgatgcacagaagcttttaattctgatgtattatttttactctttacttttgtttgtgcttttgtgtcataaccaagaaatcgctgccaagtccaatgtcatgaagctttttccctgtgttttcttctaagaatttttgtggcactagcccttaaattaagtctttgatccattgtgagttaatttttatatggtatgaaataagggtccaacttcattatttcatatgtgtatattcagttttcccccatcgaatgctcttggcaaccttgtcaaaaatcaattggccatttataggagggtttattcctaggctttccattctgccccactggcatatgtgcctgtctttatgccagtcctacgctgttttgaatactgtagctttgtagtaagttttgaaatcaggaagtgtgagtcctacaaccttgttctttttaaagatcattttggctatttgggaaaaacattaattttaaaaaattactacttgtaagatctgtgtgaaatttctaccttctgaaaatatgaattcttctcgttaaaattttctgggactctatttcctgctgttcactaaagtgtgtatttatgctcttaattatagaaagagtattcaaacgaaaatgcagttgtgaagagaatgcagtctcttcaacttgattgtgtggcagtaccttcaagccggtcaaattcaggtaaattacactatggtcaaaatctattcatttatttgtttggttactcattcactccactctctaaaagactattcaggaccatatgggaaacagaagaaactataaaagtgaacacctgccttacaggaatttataataatattcgagagtcaagttacatgtatttgaaatatgaagtaacagtgtgctcaccagtcatagagtgttctgaacaaaaggcctaatatatctgatggggagaagtgacagttcagaagaaggaaggggacatatgtgagagcaaagtccctgaggagctcggagtgacttacagccagggtacaagcagagaggctggtctcaggtcaagtagggatacttttttcactgtaacaaaagggatggcagagtataggatactgaagccgcttagctacctgatctgtggctaattacttaacctcttctaaacttcactcatcaaatcataattgctcctcctctcatagggctcttcaagaatgaaagaattcatgggacatgcttaggacagagctgctgcagatcttttttttttggagacagagtctgtcaccccggctggagtgcagtagtgtgatcttggctcactacaacctccgcctttcaggttcaagcgattcttctgcctcagcctcccaagcagcagggattacaggcatgcaccaccatgcccggctaatttttgtattttcagtagagatggggtttcgccatgttggccaggctggtctcgaactcctgacctcaggtgatccgtccgcctcagcctcccaacgtgctaggattacaggcgtgagccaccgcacccagccacagatcttattttccacctgaattgctccaaggagacaaccaccttctctgctccaaaccaagtcccttttttccttgatcatccctgcaacagccgcttttctcttgcttgctataattttcctagtactctcttactgccagcgaggagctcctattattatttgtaaagcttgtgggaagaggaccatctcctaaatgctttggactggtctcgtacttgttttctgtgtgttactttgagatacagattcatgacggcgctcagattttattttactttttaatgtttcatgacacccattctaatgttgatcatttcttctcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacaggtaaatgggtcttcgatagtcacaagcttgtctttttttatttttcagaaaatacagcaaccaagcagctctattatagattgtgggttataatggggacagagggcatcatcagctgccagatgcaaattgcccctgtggaatggaccctgccaaaccacttggtctgggagtatttcatctggggctaggattcatcacagatgcccgcaagcatcgggttacaggtccttgtgactggcaggcatctttgactctaacctggagtgcatagtagtcctatttcacccaatctctgataaatgggaagttatctgcttatttgcttttaccttgcttgctactttttgaaggtaagaaatcagtcttggatatttttgtttctgaccttatatagctcaaagactaccattagtgatgaaaaattttcttttaactcccatatctctatcaggtgaagtaaaaggtaggtgatagtagttactggcagtctagaatggaagtgagttaatgtaatatccagcatttttatcaaataatactcaccaacatcaccttaactacctgaacagcatctaccaatgatacccacaggtgtcctggagatcttgaaatcatctcaggtagaggaagctttcagaaccacagagcttagttacattttctttcaaaaggccaaattctccaaaaacgtcctatgcaaacatcctcgatttcttccataggttttatgataagatctgtagaatgcaaaatattggattagacattaagcagtatccaagtcatgtataatcctatgagcagaatgtccttttgtttccttcatgttggggataaaattttttattagtattgccatatcattttattcattcatgattaagtcaataaaaatttcagctggggccatggttggccaggtgtggtggcttatgcctgtaatctcagcactttgggaggctgaggtgagtggatcacttgaggccaggagttcgagaccagcctggctaacatggtaaaaccctgtctctactaaataatacaaaaattagccaggcgtggtggcgcatgcctttagccccagctactagggaggctgaggcaggagaatcacttgaaccctggagacagacgttgcagtgagctgagatcatgccactgcactccagcctgggcaacagagtgagattgtgtctcgaaaaaaaaaaaaaaattccactggctgggcacagtggctcacgcctgtaattccagcactttgggaggccgaggtgggaagatcacgaggtcaggagattgagaccatcctggctaacacggtgaaaccccgtctctactaaaaatacaaaaaaaaaaaaaaaaaagctgggcatagtggcgtgcgcctgtaatcccagctacttgggagggtgaggcaggagaatcacttgaacccgggaggcagaggttgcagtaagccgagattgcaccactgcactccagcctgggtgacagagcgagactgtcgcaaaaaaaaaaaaaaaaaaaaaaaaattccaaaactcttcccactttgtgacgtgcagttctccaggcactggagctataactgaacaaacagcctgagtcccttccctcacacagtttaaatcctagtggacagaaggcagaccctaaaccaatatttaatgtgtcattttttatcttttttctacatgagcaacccaaatacacccgtttagtgccagaaatctctttgtgttttcacctcgttctggttcctggttctgccagtgacagatgtacagggatgcagcttcttcagggttctgagatgctttcagtgggaaaggggcgcttggatctacaatagataatagaattttctgtgtttctgctcccaaagatgatcctgccaggtactaagctagtaaatgaacacgctgagtttctccttgcaaatttaaataagtcttctgcttgcttttatcatcaaaaagtcctaactctataccaaaatatgagttttctaatttgccataccatagaagaagggtgtgtatgcagtaagtacaaaaggaaaataaatgtcttcctttcagtgaaagagctcttagacccactgctttttattcactgtgctgcttgaagtgaggttagaaaaggaaagctcactgggctaagagtcaggagacccaggtctagtcctgcttgtgccggtcactatatgggtgactttgggaaagtcatttgcctctccagagttcattctcctcaattataaatgaaaatgttacaactgaaaatttatatattatcagctctagtatgtttctagtattgcattcgtttctccacttcgtaactttcatggtttttccagtggtcagatttgggcagaaagcactgattcaagaacacagatttccaagtagagaggtgcagggtaaataaacctccatgtaagcccgcggtgagcgcagactgtgagtgacgaacacggtgtcagtgtaagctggaggtggtgggccaggaggattaaacagggtaaggcagcttaccactgtgcctggcgatgcgcagagtaagtgctcagtaaacggtagctgctgttattagcactgttattatcattgtcagcaaagaacacctagacagccttgagcactggaaggcacctcagagactgtcacaggggttccttgctaggattccaaggaccccagtttcagcagcttcacgaacaccctgaattgcacacagatggcgtgtgtgtgcacatcctatatttcttacacctttcagtagagagggtccctagtttccttggcttcctcagagaaaatgtaacaaatgagcacagtgcctggtccagagggtggcctaccttagggccaggtgtgctgacggaggtaccttgagctgatactcatttccctccctcctcagaactgtcttctcaaccaccttctctctcctacaaagatatgccaagtgctctttagggacagtaccaacacttgacaaattcagctcagcatgaatgtcattgagcccatgccccgtaccaaggcccaggctagcgagtgtaaggagtacaaaaacaagtgggtgtggtcgctgcccttgggactcacattctagtggagaagtcaaacagacccagctcagatacagagcggcgcagggcagagtgctgagtctaggattacagtgcaggctcatggaaggaggtggaatttgtgctgggcctatgtcagaaactcacaagcagggagaagcacgtggggaggtcagcagtccaggcagaggcaggagcatcatgtcagcagatcggtgggaaatctggtgggactggagtctctggagtggagacagggaggaaacagtgaaaggaagatcagagacaagagggtagggcagagctgtggctagcttctgaatactgaacctgagaggtttgagtttatcaggtaaaggaggaggacccttcacagatgctgttcagagtcgtgggatcccgctgggaagtgtggctgagctttgctgtgttgttcatccacttctgttttctgacttggtgttagccacatttttttatggtaaaattcaaaaacataaaatttaccatttcaaccatttttacagtggcattatctaacattcacagtgttatgcaaccatcaccaccatctatctatataactgttttaatcttgcaaaactaaaactctgtatccattaaacaccaactccccagctcccctcccagccccggacaaccaccattctactctctgtttctgtgactctgaccagtctaggtacctcatgtgagtggaaccatgcagcatttttctttgtggctggcttcattcacttaggaaatgtcctcaagcttcatgcatgttgtagcatgtgatgtgattcccttccttttcaaggctgactattccattgtgtagctagaccacagttgggttatccactcatctgctcatgggtgcctggcttacttccaccttttagctattgtgaataatgctgcagtgaacacgtgggtagaaatatttcttcaacaccctgttttagttcttttgggtatatactcagaaacggaattgctggatcatatggtaattctatctttaatttttttgaagaatcacgatttttttttttttttttttttgagacagactctcactctatcaccaggctggagtgcagtggcacgatcttggctcactctgcaagctccgcctcctgggttcaagtgattctcctgcctcagcctcctgagtagctgggattacaggcacccaccaccacgcctggctaatttttgtatttttagtagagaaggggtttcaccatgttggccaggctggtcttgaactcctgacctcaggtgatcacctgcatcagcctcccaaagtgctgggattacaggtgtgagccaccgcacctggcccactatactctttataggagcagctgcaccatttcacattcccaccagcagtgcacaagggcttcattttttccacatctttgtcaatatgtgttatttctttttttttttttaatcataggcatcctaatgggtatgaggtggtttagccagatttgaaagaagaaaagtttaaagggtggcataggataattaacttaatgtttggcataaagccctgcttttttgccatgctgtatttttttctcttgcttttgatctagaattacttacctgtgtgtttctctctatgcagatgaatctataaaatataccatatacaatagtactggcattcagattggagcctacaattatatggagattggtgggacgagttcatcactactagacagcacaaatacgaacttcaaagaagagccagctgctaagtaccaagctatctttggtaagataccctggaaggactcaacgcctagcaaccttgaactttcttacttgtgagaaggacatagtggaatcttaaaatttctctgataattcttcttgaaagttttcttatgttacttctgcaaaatatctttgtaatttccatataattcctttgtcatttatttatttttaatatggagataatcacagtatttaatttatggatttgttgtgaaaattaaatgagttgatatgtataaaaagcttagaacactgcctggcacatagtaagttctataaaaagtgttaaactttcatcatcatcatcatcagtattacttctcagacccagaagaaatagctgtgtcaggccctcatcgaaggagatcctgtaaaagatagaagactgtgccctccacagcacttctgtaataaaatcaagagggctatctcttatagggttctatggtgcagcccataatgctaatcccaaagtacagtctggaaaaagtcatgaggccaaaacgacttgattaagaactcagctttctaattaaccatgggattggcgctttttttttttttttctgtaaaggaaccaatagtaaatgttaggccctgctggctttgaggtctcttggaactactcacctctcttattgtagtgcaaaagcaaccacagacaatatgtacatgaatgagcacggctgtgttccaataaaagtggatttacaaatgctggcaggagtcagatttggcccacaggccatagttggccaacccctggtctaacccatttgaaggacagcttttaaaatacctagaacagaacttgcgaatggtcaggggcacctgtgagaatctaatgaaacccaggatctaccccctacagaaatattgacaaaatttggaaaatatcttgggaagttcttggactgctgacaatctataagatatggatagagagagagccacatcacagagggctgaggaaagaagagaggtgggaaggaagaatgaaatgttttcttccttatattctgtttaatgaggtagtgtggggattaaggaattttttttgttgtggttgttgtacattttcatttgtttccttttattatttttatgaagggcgaagggaaggaaccagtagaaggagagaggttatggctattggagaagaggcaatgtcggtcggcagtggggtggagtaaaggaggggttagggtccaaaaacagaccttgaagagtatagttacctcgtcgcttgaaagacaaggaaggctttgtaactgaatgggttcacgcctgctggacaattccttgattaagtaagaggaaagtgaactgttgggccggggtggtggtcaggagttgagcggctttaactctggccagatgtaagcggagaatgtctaaaaggaacaaaaatggtggttttccctttacacccaggaaggtctaataccaacaggatcactggctaagattgactagaacacccaacctcgtagccacacaggggttttgttcacagagccatcagctgtggctcagggatgtgtctactaggaggaaggaatgccttattcttttttgttttgtttttgagacaacatcctgctctgtggcccaggctgaagtacaggggcacagtcatggctcactgcagcctcgaactcctgagttcaagtgatcctcctgtctcagcctcctcagtaactgggactacaggcatgtgccaccatacccagctaattaaaaaaaattttttttttggtagagacaggttcttgctgtgttgcccaggactggtctagaactcccgggttcaagtgatcctcctgccttggcctcccaaagtgttgggattataggcatgagccaccacgccaggccgggaatgccctattctaattcacataaagactccatgtgggctagcaggcatgtgaaatgtctgccagtgcatcaacagctatataactaagaagttatctttttcactgtcttctggtactcttcatttcacttgggttttttagcaattcaggaagctggaatgtttgaatgggttttagcttgataccttcttctttttcccatttggcagataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaaccctggatgggctacggcagctgaagtggacgcctcacttagtggataaccccagaaagttggctgcctcagagcattcagaattctgtcctcactgataggggttctgtgtctgcagaaattttgtttcctgtacttcatagctggagaatggggaaagaaatctgcagcaaaggggtctcactctgttgccaggctggtctcaaacttctggactcaagtgatcctcccgcctcggccttccaaagtgctgggatatcaggcactgagccactgcgcccagccaacaatccgctctgaggaaagcgtaagcaggaagacctcttaatggcatagcaccaataaaaaaatgactcctagttgtgtttggaaagggagagaagagatgtctgaggaaggtcatgttctttcagcttatggcatttcctagagttttgttgaagcaagaagaaaaactcagagaatataaaatcaacttttaaaattgtgtgctctcttcttcacgtaggctcctgttaaaaacaaagtgcagtcagattctaagccctgttcagagacttcgtggatcacagctgcagctcaccgccacatcacaggatccgttaacgttaatacccaatactctgtcagccactgtaggctctaagaaccacgtgcagtcttcagcccattaaattatcgattattttttaatgaattgaatttatattgagtcttcaaattaactgaatggatttaaaggggtaccaaggaggggggaaacatcagaatttcccaggcagttgttgcaaggaattggtactaaccgtgactacaacaaaaattcttgattgacttttaaagttatttcctggcattctggtaccttcacccagcctgagtgccctggagagggaacaggaaatgctgatctctacccctgggtgagaccagaacctcagggctgatactgttgagtggcttcctcggtttactctgtgtactgtgaaagtattttcatattttttctgtgtgccagagtgaaaaaggacagcttctgagtgtggtaattgtgcctctagcacccagcctttcaaagcccacctgaaacctgggggtggatgaaagaactagaatagaagactgaagctgggtaggccgctcagtgtccactggcattttgctaaaccgacaaggaaggctgtgtgcttagctctccccagagggagggcgagaagggtgtggtgatggtcaatctggctgtcggaacagattctggtgtcttgggctgataacagtgttgttgattctgattgtgaatcccctcaactctagcagacacatacacacccctgaaatggggctgcagagcaggctgtctcagccttgccactgtcggcatctcggcctgggtaattctgttgtggggactgtcctgttccttgtaggatgtttagtagcatccctgcccccacctactagatgccaggggcactgttctccccagccccccgccccagttgtgacaatagtctctaaacattgtcaaatggtccaaggaaaggggaaaattgccccggttgagaagagcactgctgtaaagtaatgagcctcggctctcctgtctgcacctgtccggttactacttggccaccacgcagccttggctcctacagcccaaaagggagaatggagggaggctccaggctttgctggaggggcctgggtgagttctgtttgctccttgtaccaccatccaaatggtgttatcaaatctcttagattccaaagaggttgaataattaatgttcaaaggcaagagggcaaggcattttttaacactttttaaaataaaaatttataccacaacttta

SEQ ID NO: 10 - DNA, 1001 bp, Homo sapiens
atttactgagataagaaaactgaggacgaagcaggtctggggtgaatcaggtgctatccctttggacagtcacatgactggtgtctgttcagcacccaagggaccatcaaagaggctgttgtggagagggaatccgaaggtcagggccagagatagaaatctggggagtcatccactatataggtgatgggttaaagccttagaactcttcttttcatgaatcattgttgttctttaggttgtaatgaagttttaggcctcagcttccctgaactggagtgtttttccttcaccttttccggtctctgggttgcatcgccagactgtctctaagcccaacaaacgcgttccttccaggcaaaagcaggagatgacacacaccatgagccagattttccatggcagacttgtaaggaacaagataatctcattgttcctcctgctacttaaaataaaggtaatattgattttatagtagcagttcaggtcctaaggcatgayattgattaagtgtctgatgagaatttgtagggtagtctcccagacctgcagctacagggcatctccccactgggccaggcctctgtgctgacctccactgttataagtggtgtttttcttaggaatccttagccctgtttagtctacgcactgtccagaagcatttatttaacctgatggatggctcaatccaacccatcaccttaatctgaccttaaagatgggaacctaatgtttttgttcttagtaaatgtgaagactaattttgacactgtttagaattattttcttattagtagcagtagatttcatgaattttctgctgtgaaatttcctattaaaatactataatctaatgttaaatgacgagttaatgggtgcagcacaccagcatggcacatgtatacatatgtaactaacctgcacgttgtgcacatgtaccctaaaacttaaagtataataaaaaaaataaaaaataaataaaatactataatctgact

SEQ ID NO: 11 - DNA, 1001 bp, Homo sapiens
aaaaactctgtgcctaagtgatcaaaggaataaacaaatgagaaagaaaacagactattaaaagcaggcaaaaggatagcagagctgaaaagttagtaaaggacaagacactctgacatcagtgattagaccttttggctaaaattcagttccagcagggggaaaatgaaagataagcctggaatgatagattgcaccctgattatggaaggtcctgaaagtcaagctaaggagtctggattctatcctgcatgtaatgaggagcctcagaaagcttttgagaaggacaggagcacagtggaaggatgtttgttatttgttgttctgatttttaaacaccaatctacttataatatggttacggtgaaagaaaccaaaagccagataaggagactactgcaaaagcccaggcagacaggagctggacctgaacaaggagacagcaatggaaatgaaaaggaatcaagagttacaggtaggcaggtggatctgaaatctcabatagtaccaagacacgtgactaggaaaggtggggccatataagaaataaggaagtcaatgtcattaacacatctactgagggcccactaagctagttgctgtgctggacagaaaatgcaatatgagttggaggtaaagatattttattttaaacatgctgggggaagaggtagatatggtggcactcagcacccttccaatctcctcttattgtgccttcctatggtgcagaggatgaaaaggtaaacactattatttctcagacccttttgcagctaggagttctggatgagaaataagcttcaccaatttaatggcggcatgtgagaatcagaagtcaaaaataaaatggaggccatcaccttgctgtttcaactttttctgcaggcaacatagtcatgaggcaataacattccagaatctggccattcactttgtagttctcaaatgccagttgtaatggtggtaccttcctgatcccaactacaactatggttgtg

SEQ ID NO: 12 - DNA, 1001 bp, Homo sapiens
taggacctcctatttgggacattaaggaaagccttctctaaaaaaagaacccattttgagcaacatgcgaaaattctttgagtcagttggcctcatttgtaagcctttgattgctttatttgtttgtttgcttgcttgtttatttatttatatttggaaggcaatctttctgcttcctgactggctgtcagatctcccaaataacatcatccctttgctggtgtgaatatagcctagacttgaggcagcaattaactgatcatgtaatagaagaatcccaatcttccagtttctttttttaacttcttgggtggttggggcacatcattgcaacaattattacatttactcaagagtttgtcttttctattaaaaaaaaaattccatctaagtgccttacggtgaagaggaggagattgtgtaactggagtctccccttaactggtctttattgttaaagaagagtgatccctttgtgtttcagcttggcacacagaaacdgttttaatttaacagtccagctcctttaatagatcaattctctattgtggtttgaatttggtgcactcccaatttactctaaacttctacgggcttccttggagaaactggggcagagatgccgaagactcatttcgggtacaatttagagtttgtttatttatataaatgtaaggggtaccagtgcagttttgttacatggatatattgtgtagtggtaaagtctgggtcagcttttagtgtaactcatctgaataatgcacattatatccattaatttatagttttatctcaaacctcttattaattaatgctagccatggaagctagctagcgcttgtaaatgcctgctgtgtgttagatgctctagtaggtgttttagacaccctcatctaatcttcatggtgattttctgatatatcctgaacctgtacaattttctccacttctgtcactactactacaggccaagccaccatcatttcttgcacagacttctg

SEQ ID NO: 13 - DNA, 1001 bp, Homo sapiens
tgtgttaggcgagatatggtgagtggtttcagaggcttgtgtgagaaagttgatacactgcccctaccccatgcaaacacacactttcttttcttatgatgacctttctttaaaagcatttgttggaatatgagattataggccataagtgagaaaacaactctttttgagctgtgaggaatactaggagaggagaaagtgagctgtgtgtgctgcccatggtggtacgctgctatggttctacagttccagtcatttttgacagcatggattcaatgcaaaatggcaacacacactctgtatcttttggcagatcagaacataatgaaaataaaataaaaaaattcaaaactggctcttgaatgaaataggattcagaagagatgatctcaaatctactttatgagataatgtcctttttaaaaataaacactaacatcagttatgcatttagaatgtctgaattattattctaggttccttgcgactgctgtgaatttwgtgatgcacttggatagtctctgttactctaaagttttaataggtaacagtcagaaatggagtgggagagcataaaagcaaactgaaatgcaaatagctggtaccctgaagccattaactttaagctggttattcctgacctactgtttggacataagatggtagagaggctgagtgtgacttgaacatttgttccttagaaacaccatccttgggctgggcgcagtggctcacgcctgtgtttccaggactttgggaggctgaggcgggcagatcatgaggtcaatagatcgaaactatcctggctaacacggtgaaatcccatctctactaaaaatacaaaaaattagccaggtgtggtggcgggtgcctgtagtcccagctactcgggaggctgaggcaggagaattgcttgaacccaggaggcagaggttgcagtgagccaaggtcgcaccactgcactctagcctgggcaacagagtgagactccttctcaaaaa

SEQ ID NO: 14 - DNA, 17 bp, Artificial Sequence
  forward and reverse primers, respectively, for 28S
tgggaatgcagcccaag

SEQ ID NO: 15 - DNA, 24 bp, Artificial Sequence
  forward and reverse primers, respectively, for 28S
ccttacggtacttgttgactatcg

SEQ ID NO: 16 - DNA, 20 bp, Artificial Sequence
  forward and reverse primers, respectively, for RIPK1
agactgctcgtcaagtgtgg

SEQ ID NO: 17 - DNA, 22 bp, Artificial Sequence
  forward and reverse primers, respectively, for RIPK1
ttgagctgtagcctgaacctta

SEQ ID NO: 18 - DNA, 29 bp, Artificial Sequence
  forward and reverse primers, respectively, for RIPK1 promotor
gattacgcgttgcagttccaactttgctc

SEQ ID NO: 19 - DNA, 29 bp, Artificial Sequence
  forward and reverse primers, respectively, for RIPK1 promotor
gattaagctttggcttcttcaagtgtaag

SEQ ID NO: 20 - DNA, 38 bp, Artificial Sequence
  forward and reverse primers, respectively, for rs5873855 element
gtaaaatcgataaggatccgggcagtaagtacagtccc

SEQ ID NO: 21 - DNA, 38 bp, Artificial Sequence
  forward and reverse primers, respectively, for rs5873855 element
aaggctctcaagggcatcggtcaaagccacctaagtgc

SEQ ID NO: 22 - DNA, 32 bp, Artificial Sequence
  forward and reverse primers, respectively, for rs5873855 SDM
tatatataagtggaatcatacagtatttgtcc

SEQ ID NO: 23 - DNA, 18 bp, Artificial Sequence
  forward and reverse primers, respectively, for rs5873855 SDM
aggcacccagtgcagtta

SEQ ID NO: 24 - DNA, 23 bp, Artificial Sequence
  forward and reverse primers, respectively, for rs5873855
gtcccatagtacattcactttcc

SEQ ID NO: 25 - DNA, 21 bp, Artificial Sequence
  forward and reverse primers, respectively, for rs5873855
aagccagacacaaaaggacaa

SEQ ID NO: 26 - DNA, 1001 bp, Homo sapiens
  misc_feature (501)..(501): n is a, c, g, or t
ctttttctgacacagattggttttgacttctgaaccgtcatataaatagaattacatagtatgtatgcttttgtataaggcgactttcacttatcacgtttttgcatttcgttcatgcggttttcattcatgtgtatcagtagttcatttttattgctgagtagtatcccatcgtgttcatacaccagtgtgttcactcattctcttactgatgggcagctgtttccagtttggagctattataaataaagttgctatgaacattttacaagtgttttccaacatacgttttcatttctcttgggattggaattgctgggtcatagtgcaggtgtttgtggttttataaacaaaataaatgcctatgaaatttcaatgaatattttatgccccagaaagagtatttttcatggtctgtatctggatgtagatatttattattttattttattttaatttttttgagacagagtctcactttgttgcccaagctggagtgcnagtggcatgcgatctcggctcactgcaacctccgcctcccaaatttaagcaattctcctgcctcagcctcctgagtagctgggattagaggcgcgtgccaccacgcctggctgatttttgtatttttagtagagacggggtttcaccatgttggccaggctggtcttgaactcctgacctcgtgatccgcccgcctcagcctcccaaagtgctgggattacagacgtgagccacggcgcccagccgggatgtgggtatttctggaatacaaaacatccatagccctccttttactctcaaagcaaaacacagacacgcacattcacatgcacacaccagtgtgtccgtgtcctgttggtttgcatggagtttctgttttgccacgaagctttttgccagcccttttcccaaagtgtttgtaccattttatactcccaccagtgatgtttgagaggtctggttgcttcacatgtttgccagtgtttggtgtattagtcttt

SEQ ID NO: 27 - DNA, 1001 bp, Homo sapiens
cacagtgaattttttcttttgtgttttataattttggaaagggagctcatgttaaatttgtatttgtaggaattctgtctggcctggcttgaggttgttgtttgctttagcctataacctcaggatggcatcagcctgtgaccacttcctgtgtaaaagttcatagtttaggcgatcctctcaattcaaagtctgtatctatttcaagacaggcccaaggttataaatttgcacaaactttttttttttttttttaataccttgtgcccagtccaagacaaatatgtttgtttgccatgggctgctttttttctagttcatcctttcatcaaagggttgcacattgagcagtgccgtgttaaggggggattcttcattctaccttcttttcttgtattccttgacctcagctgggctttaaaccttgacttctggatggcgaggttggcactcccctgtgtgagctactgcctgcctccggtgctctgtttctgtccctavagttcttttcctttccacggagttttggtatgcatttcaatgaatgtttgtattatgttgtccaacatacttgtgtcctttatagctggagggttcttaaaattttctaatgctccatattgccagaaataaaaaggctgagggaataaatattatagtcttctctgattcttaaaaatatgaaaaatagctcatcagatgactgtaagtctatcctcagaaatccaaggcacaccaagcatgtggtggaatgttaacaaaagtttttaaaaatgatcttttctgccaggtacaatggctcacacctgtaatctcagcactttgggaggctgaggtgggaggatcccttgatcttaggagtttgagaccagcctgggcaacatagtgagatcccccggcccccccatctctacaaataattaaaaaatcagctggtcacggtggggtgcacctgtggtcccagctactcaaggaggctgagggaggaggattgcctgagc

SEQ ID NO: 28 - DNA, 1001 bp, Homo sapiens
ctctactggttgctttaaagggaaggctctcagaacctctcaggaatgaactgccttttcatttttatacttagggctggtcctcactgaaaatgtgaagcgagtgaataggtctgtcttcacccatcctcctgtttccttttcattgtccccccaccacacttatctctttgtctttctctatttccttcttgttcatgtctcttctgttacttaccagctgtgagagtccttcactacccagtctcagagtccacatcagcgacaataacaatccgaagagccatcgtcaccctgacatcttggcaggctccctctcactctacccaccaacggctccgcagcagccaacggctccacaacagccaacgtccccatcccgctcagaacttagcccacccacccctgctccctactcactcaatgcagttgggccccttgtacactgttttcatgatcatgagtccctgggttctgtggaaacacagagacaccttcccvaagcctccgctgtccagttctgcactctccaggaagtcactggatttcatcttaatgacattcaaggacatgtctggttgcattctgaagctcaagaacgcccaaaatggtaccactttttccccccccggcagagctgtaccctgtaaagaaaacagagagaaaacctcagggcaagactatatatatatatatatatatatatatgtttttttttttctcctttggagacagggtctcactttgtcagtcaggctggagtgcagtggcacaatcatagctcactggagcctcgacctcctgggctcaggcaatcctcctccctcagcctccttgagtagctgggaccacaggtgcaccccaccgtgaccagctgattttttaattatttgtagagatgggggggccgggggatctcactatgttgcccaggctggtctcaaactcctaagatcaagggatcctcccacctcagcctcccaaagtgctgagattacagg

SEQ ID NO: 29 - DNA, 1001 bp, Homo sapiens
ttcattgattcattcattccttcattgatttacatttttggagcatgcactgccagttactgggcaggagtactgtgaggaagtgagaaaccgctctgccttctaacgcttctggcctgtgcctggaagagatcggtacagacccaagcatgaccccagcacgtgggagtgatgtgttggaggtgtgcaccaggctcttgaggcgctggctctgccagcctcagcatagcacctttcctgcccacaggcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgaggtagagagggcccctccgcacggggatccccagcgcttgggccctggctgtctgttatggctccccttggggtgtttgtttgcagctcgttagcatmaaatttgcaaattgcttggtagtttgaattttcttttcctttttttatagagacaaggtcttgctatgttgcccaggctgaactccaacacctggtctcaagtgatcctcccacctcagcctctcaagtagctgggattacaggctggatttcctgaagtcagtttctccttaaaacatctatgccttgattgggctccgtgggtcacttctgtaatctcagcacttttggcaggaggatcacttgagccttggtgctcaagaccagcctggataacacagtgaaatcccctctctacaaaaaataaaaggtgcatgtgaacagagcaagaccctgtctctgaaaaaaagagaaaatcttccttgccttttactgccctgagagtggtctcttcatcctctcgatcctgtggacagtcctccatagtctgtgtcttaggaatgtgattggctgtttaccatacctgtccgctgctctgcaatctacagttcagagacaag

SEQ ID NO: 30 - DNA, 1001 bp, Homo sapiens
  misc_feature (501)..(501): n is a, c, g, or t
cacacgacatcatactaagtttacattaaggacatgaacaatgccatgcaaatgtcgcgtgtaaaataacattaagagaaaaaagaatacaagggaacatgttcacactgattacaatcatagaaaaatctgagataaacatgatgttaaaactacgcttaagtgaattaatatctatttgatgacctggtagcagatacttcaggcttctgtagagtctcaggtcactggttcttataattgtattcttgttatgtgagcatcaatactaacagaaacaattccagtttctattatagtggttaaattacagttttgcttaacaataaaaaatcagtatacaaacaagatgtttcctaaaaatgtatatgtattttggtcgttggttaacaggaaaaaaattttaatgatagaagtgagattccccctcaaaccatatttatgacataacgtagcagagctacactgttaatagcttgatcaccaccatacagaaagtancagaaaagatgttttctcttcttccttggtatacctttggacttaggaaaaacttctgagtaagcaaagttggtccccctctctcctctctttctgcttctctctcaggtcctcctgatgccctgactgagtccaaacaggtgcacctgaactttccctcatcttgcacaatgcctcatgtccccaatgatttctctctcatctcagaagctaagcagggttgggcctggttagtacttggatgggagaccaaagacttatttcccaatcaaaatgtttacttcaaacagctgatcttctctagttctggggtggagagcaatctgatcaaactataataccatgcgtataagtgatcacctcttctccttcccaagggtatttttgtgagatgtctgttggcttggcaattgtggggtgggagaagaaagtggaaaagtgggagtatttgccataaaggcagccatatcccgccacccctccccataccctgtgcaatc

SEQ ID NO: 31 - DNA, 1346 bp, Homo sapiens
cttcttgggtgatcatcagctcccatggtttgatgatcatctctttgcaaatgcttcttaaatctgtgtctctggctctggaatggatatccacttgtcttacagtcagatccatcgcaggtaccagaggaacctccaaagcagcatcccccaaacacatcctgtcgacactgtcctgcagctcgccccgcttcacccctcttcatatgcttgggaagtggcatcgccatccttttaccaagttgttgcccaataagaaacttcagagtcatcccagactcgcccttgcccctctctcccacattctatcagatctcaaaacctgtgaattcaagatatatgtatgactcaagatatatctgcaacccatctgcctttctctgtctccactggcctctgctcagttcaaaccctcctcgtttctgcagcaggctccttccaccgtccaccattcatgtggcagccaaaatggtcgtcctaaaaggcacatgcaaccctctccctcacatattggaaatccttcccagatgctctgtggtcttcaggatgaagttcgaattcctcagcgtggaataaaaggccttcatgttctggcctttgcttctctactggcctcgtccctaaccctcccttctttgtcagtgtgcagttacactgaggaatttgtgggatgcgtcatgatctctgtaacttctagtctgttgatatcctgcttctcttcctggagtcctcctccttaagtctccctttcccattctttacctgctaatttttctggactcaacaaagccttcctggacctttttagtcaggtttgtgctactggaaccccttgtacatccttttttttttttttttcccgtgagacagagtctcattctgtcgccaggttggagtgcagtggtgcagtctcggctcactgcagcctccacctcccaggttcaagcaattcttctgcctcagcctcccgagtagctgggactacaggtgcacaccaccatgcccagctaatttttatatttttagtagagacggggtttcaccatgttggccaggatggtcttgatctcttgacgtcatgatccgcctgtctcaacctcccaaagtgctgggattacaggtgtgatccaccatgcccagcctcakccttcttgaatagcaatgatcactctgtgttttaagtcactctcattaaactgagctcctaaagacaaggactgccctgtatttgcttttgcacatggagcatgccattggccttgcccacataatctttttgttccatcagcaggcagctttgtcgagaattttgttttatctgcagacattaagatattgtttggtca

SEQ ID NO: 32 - DNA, 1001 bp, Homo sapiens
  misc_feature (501)..(501): n is a, c, g, or t
gatgacatcactgagtactgcccaagagaaattatcagtctcatgaagctctgctgggaagcgaatccggaagctcggccgacatttcctggtaagagcatcttttctgactgtgtaggatgcatcctgtgtggtgattttcctagtaacatattgttaggaacttggtttgaatcctcagaaaactgagttcgacttgacttgtggttttaaactgattttctagatttcaaacatgttattcagtgcaaagaagccctctctctcaaaagcatgatggcacataaggccttcttgtttcctttttttgtaattaactttttaacttgggtttaaatatacgtaacatgaaatttaacattgtaactgttttcaagtgcactgttcaggggcagtaagtacagtcccatagtacattcactttccatcgtctctctccctgcccctggcagcccctatcctactttctgtctctgtgaatttaactgcactgggtgcctntatataagtggaatcatacagtatttgtccttttgtgtctggcttactccacttggcatgatgtcctcgaggttcatctgtgttgtagcatatgtcagaatttctttccttttcaaggctgaataatattccattgtatgaatggagtgcattttgtttatccattcgtccgtctatgggcacttaggtggctttgaccagccttcttgcttcttcacttcttttatttccatcccctcccttgtttagctggcatctttagaaagtattttgccgtctagattggccttgtcatacagggatgaggaaaggtgccgggatgtttgctgagcagtattctatggtgtggatgtaccacggtttggttaactactcactccttgaaagacttgtttccagttcttaactgttgggactaatgcagctgtaaatattcgtggacatgtttttgtgtgaatataagtcttcatttctctggagtaatacccaggggtacaata

SEQ ID NO: 33 - DNA, 1001 bp, Homo sapiens
  misc_feature (501)..(501): n is a, c, g, or t
tctctctcaaaagcatgatggcacataaggccttcttgtttcctttttttgtaattaactttttaacttgggtttaaatatacgtaacatgaaatttaacattgtaactgttttcaagtgcactgttcaggggcagtaagtacagtcccatagtacattcactttccatcgtctctctccctgcccctggcagcccctatcctactttctgtctctgtgaatttaactgcactgggtgccttatatataagtggaatcatacagtatttgtccttttgtgtctggcttactccacttggcatgatgtcctcgaggttcatctgtgttgtagcatatgtcagaatttctttccttttcaaggctgaataatattccattgtatgaatggagtgcattttgtttatccattcgtccgtctatgggcacttaggtggctttgaccagccttcttgcttcttcacttcttttatttccatcccctcccttgtttagctggcatcnttttgccgtctagattggccttgtcatacagggatgaggaaaggtgccgggatgtttgctgagcagtattctatggtgtggatgtaccacggtttggttaactactcactccttgaaagacttgtttccagttcttaactgttgggactaatgcagctgtaaatattcgtggacatgtttttgtgtgaatataagtcttcatttctctggagtaatacccaggggtacaatagctaggttgtatggtagtttcatgtacagtttattttaaaaattccctaacattttcccagaggggctgtgccattttatgttcggtgtccgagtgatgcaggttcttgtatcctcaccagcatggggtacccaggctcctcacacttctgcttagtcagctacaaatttatttgagtattcccacaagccccacttcaggtttggtaattcgttagaaagactcacataactcaagaaaacattttacttacgtctccccttttact